SEQUENCE LISTING (50 sequences)

SEQ ID NO: 1 - protein, 22 aa, Artificial Sequence
  Synthetic Construct
  MISC_FEATURE (1)..(5): Xaa can be any amino acid
  MISC_FEATURE (8)..(8): Xaa can be any amino acid
  MISC_FEATURE (14)..(14): Xaa can be any amino acid
  MISC_FEATURE (16)..(16): Xaa can be any amino acid
XXXXXGVXAKAGVXNXFKSESY

SEQ ID NO: 2 - protein, 23 aa, Artificial Sequence
  Synthetic Construct
  MISC_FEATURE (1)..(6): Xaa can be any amino acid and can be either present or absent
  MISC_FEATURE (9)..(9): Xaa can be any amino acid
  MISC_FEATURE (15)..(15): Xaa can be any amino acid
  MISC_FEATURE (17)..(17): Xaa can be any amino acid
XXXXXXGVXAKAGVXNXFKSESY

SEQ ID NO: 3 - protein, 24 aa, Artificial Sequence
  Synthetic Construct
  MISC_FEATURE (3)..(3): Xaa can be any amino acid
  MISC_FEATURE (7)..(7): Xaa can be any amino acid
  MISC_FEATURE (13)..(13): Xaa can be any amino acid
  MISC_FEATURE (22)..(23): Xaa can be either Gln, Pro, Tyr, Ile or Leu
YKXLRRXAPRWDXPLRDPALRXXL

SEQ ID NO: 4 - protein, 23 aa, Artificial Sequence
  Synthetic Construct
  MISC_FEATURE (3)..(3): Xaa can be any amino acid
  MISC_FEATURE (7)..(12): Xaa can be any amino acid and can be either present or absent
  MISC_FEATURE (21)..(22): Xaa can be either Gln, Pro, Tyr, Ile or Leu
YKXLRRXXXXXXPLRDPALRXXL

SEQ ID NO: 5 - protein, 22 aa, Artificial Sequence
  Synthetic Construct
IKLSGGVQAKAGVINMDKSESM

SEQ ID NO: 6 - protein, 22 aa, Artificial Sequence
  Synthetic Construct
IKLSGGVQAKAGVINMFKSESY

SEQ ID NO: 7 - protein, 23 aa, Artificial Sequence
  Synthetic Construct
IKLSGGVQAKAGVINMFKSESYK

SEQ ID NO: 8 - protein, 17 aa, Artificial Sequence
  Synthetic Construct
GVQAKAGVINMFKSESY

SEQ ID NO: 9 - protein, 17 aa, Artificial Sequence
  Synthetic Construct
GVRAKAGVRNMFKSESY

SEQ ID NO: 10 - protein, 17 aa, Artificial Sequence
  Synthetic Construct
  MISC_FEATURE (11)..(11): Xaa is Nle
GVRAKAGVRNXFKSESY

SEQ ID NO: 11 - protein, 24 aa, Artificial Sequence
  Synthetic Construct
YKSLRRKAPRWDAPLRDPALRQLL

SEQ ID NO: 12 - protein, 24 aa, Artificial Sequence
  Synthetic Construct
YKSLRRKAPRWDAYLRDPALRQLL

SEQ ID NO: 13 - protein, 24 aa, Artificial Sequence
  Synthetic Construct
YKSLRRKAPRWDAYLRDPALRPLL

SEQ ID NO: 14 - protein, 17 aa, Artificial Sequence
  Synthetic Construct
  MOD_RES (1)..(1): ACETYLATION
GVRAKAGVRNMFKSESY

SEQ ID NO: 15 - protein, 17 aa, Artificial Sequence
  Synthetic Construct
  MOD_RES (1)..(1): ACETYLATION
  MISC_FEATURE (11)..(11): Xaa is Nle
GVRAKAGVRNXFKSESY

SEQ ID NO: 16 - protein, 24 aa, Artificial Sequence
  Synthetic Construct
  MOD_RES (1)..(1): ACETYLATION
YKSLRRKAPRWDAPLRDPALRQLL

SEQ ID NO: 17 - protein, 24 aa, Artificial Sequence
  Synthetic Construct
  MOD_RES (1)..(1): ACETYLATION
YKSLRRKAPRWDAYLRDPALRQLL

SEQ ID NO: 18 - protein, 24 aa, Artificial Sequence
  Synthetic Construct
  MOD_RES (1)..(1): ACETYLATION
YKSLRRKAPRWDAYLRDPALRPLL

SEQ ID NO: 19 - protein, 23 aa, Homo sapiens
KCLRREAPRWDAPLRDPALRQLL

SEQ ID NO: 20 - protein, 23 aa, Rattus norvegicus
KCLRKKTPRWDILLRDPAPRPLL

SEQ ID NO: 21 - protein, 23 aa, Mus musculus
KCLRKKIPRWDMFLRDPVPRPLL

SEQ ID NO: 22 - protein, 13 aa, Homo sapiens
  MISC_FEATURE (1)..(1): Xaa can be any amino acid
XLYENKPRRPYIL

SEQ ID NO: 23 - protein, 18 aa, Artificial Sequence
  Synthetic Construct
  MISC_FEATURE (11)..(11): Xaa is Nle
GVRAKAGVRNXFKSESYC

SEQ ID NO: 24 - protein, 18 aa, Artificial Sequence
  Synthetic Construct
  MOD_RES (1)..(1): ACETYLATION
  MISC_FEATURE (11)..(11): Xaa is Nle
GVRAKAGVRNXFKSESYC

SEQ ID NO: 25 - protein, 123 aa, Homo Sapiens
GVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSED

SEQ ID NO: 26 - protein, 31 aa, Homo Sapiens
GVKIYSFGLGGRFLFASVMADKDTTRRIHVS

SEQ ID NO: 27 - protein, 31 aa, Homo Sapiens
ASVMADKDTTRRIHVSTDQGDTWSMAQLPSV

SEQ ID NO: 28 - protein, 31 aa, Homo Sapiens
STDQGDTWSMAQLPSVGQEQFYSILAANDDM

SEQ ID NO: 29 - protein, 31 aa, Homo Sapiens
VGQEQFYSILAANDDMVFMHVDEPGDTGFGT

SEQ ID NO: 30 - protein, 31 aa, Homo Sapiens
MVFMHVDEPGDTGFGTIFTSDDRGIVYSKSL

SEQ ID NO: 31 - protein, 31 aa, Homo Sapiens
TIFTSDDRGIVYSKSLDRHLYTTTGGETDFT

SEQ ID NO: 32 - protein, 33 aa, Homo Sapiens
LDRHLYTTTGGETDFTNVTSLRGVYITSVLSED

SEQ ID NO: 33 - protein, 21 aa, Homo Sapiens
GVKIYSFGLGGRFLFASVMAD

SEQ ID NO: 34 - protein, 21 aa, Homo Sapiens
GRFLFASVMADKDTTRRIHVS

SEQ ID NO: 35 - protein, 21 aa, Homo Sapiens
DKDTTRRIHVSTDQGDTWSMA

SEQ ID NO: 36 - protein, 21 aa, Homo Sapiens
STDQGDTWSMAQLPSVGQEQF

SEQ ID NO: 37 - protein, 21 aa, Homo Sapiens
AQLPSVGQEQFYSILAANDDM

SEQ ID NO: 38 - protein, 21 aa, Homo Sapiens
FYSILAANDDMVFMHVDEPGD

SEQ ID NO: 39 - protein, 21 aa, Homo Sapiens
MVFMHVDEPGDTGFGTIFTSD

SEQ ID NO: 40 - protein, 21 aa, Homo Sapiens
DTGFGTIFTSDDRGIVYSKSL

SEQ ID NO: 41 - protein, 21 aa, Homo Sapiens
DDRGIVYSKSLDRHLYTTTGG

SEQ ID NO: 42 - protein, 21 aa, Homo Sapiens
LDRHLYTTTGGETDFTNVTSL

SEQ ID NO: 43 - protein, 23 aa, Homo Sapiens
GETDFTNVTSLRGVYITSVLSED

SEQ ID NO: 44 - protein, 6 aa, Homo Sapiens
ADKDTT

SEQ ID NO: 45 - protein, 9 aa, Homo Sapiens
STDQGDTWS

SEQ ID NO: 46 - protein, 7 aa, Homo Sapiens
LPSVGQE

SEQ ID NO: 47 - protein, 8 aa, Homo Sapiens
DEPGDTGF

SEQ ID NO: 48 - protein, 2 aa, Homo Sapiens
TS

SEQ ID NO: 49 - protein, 3 aa, Homo Sapiens
RGI

SEQ ID NO: 50 - protein, 10 aa, Homo Sapiens
TTTGGETDFT

(19) United States
(12) Patent Application Publication
(10) Pub. No.: US 2022/0000971 A1
(43) Pub. Date: Jan. 6, 2022

(54) METHODS AND COMPOUNDS FOR TARGETING SORTILIN RECEPTORS AND INHIBITING VASCULOGENIC MIMICRY

(71) Applicant: TRANSFERT PLUS, S.E.C., Montreal (CA)

(72) Inventors: Richard BÉLIVEAU, Montreal (CA); Borhane ANNABI, Brossard (CA); Michel DEMEULE, Beaconsfield (CA); Alain LAROCQUE, St-Laurent (CA); Jean-Christophe CURRIE, Repentigny (CA); Alain ZGHEIB, Montreal (CA)

(73) Assignee: TRANSFERT PLUS, S.E.C., Montreal, QC (CA)

(21) Appl. No.: 17/270,787

(22) PCT Filed: Aug. 26, 2019
(86) PCT No.: PCT/CA2019/051170

§ 371 Date: Feb. 23, 2021

Related U.S. Application Data
(60) Provisional application No. 62/722,726, filed on Aug. 24, 2018.
(60) Provisional application No. 62/804,063, filed on Feb. 11, 2019.

(51) Int. Cl. A61K 38/16; A61K 47/64; A61P 35/00

(52) U.S. Cl. CPC A61K 38/164; A61P 35/00; A61K 47/64

(57) ABSTRACT

The present disclosure relates to peptide compounds and conjugate compounds, processes, methods and uses thereof for treatment of cancer or aggressive cancer. For example, the compounds can comprise compounds of formula X₁X₂X₃X₄X₅GVX₆AKAGVX₇NX₈FKSESY (I) (SEQ ID NO: 1) (X₉)ₙGVX₁₀AKAGVX₁₁NX₁₂FKSESY (II) (SEQ ID NO: 2) YKX₁₃LRRX₁₄APRWDX₁₅PLRDPALRX₁₆X₁₇L (III) (SEQ ID NO: 3) YKX₁₈LRR(X₁₉)ₙPLRDPALRX₂₀X₂₁L (IV) (SEQ ID NO: 4) IKLSGGVQAKAGVINMDKSESM (V) (SEQ ID NO: 5) IKLSGGVQAKAGVINMFKSESY (VI) (SEQ ID NO: 6) IKLSGGVQAKAGVINMFKSESYK (VII) (SEQ ID NO: 7) GVQAKAGVINMFKSESY (VIII) (SEQ ID NO: 8) GVRAKAGVRNMFKSESY (IX) (SEQ ID NO: 9) GVRAKAGVRN(Nle)FKSESY (X) (SEQ ID NO: 10) YKSLRRKAPRWDAPLRDPALRQLL (XI) (SEQ ID NO: 11) YKSLRRKAPRWDAYLRDPALRQLL (XII) (SEQ ID NO: 12) YKSLRRKAPRWDAYLRDPALRPLL (XIII) (SEQ ID NO: 13) wherein X₁ to X₂₁ and n can have various different values and wherein at least one protecting group and/or at least one labelling agent is optionally connected to said peptide compound at an N- and/or C-terminal end, for use in inhibiting vasculogenic mimicry and/or for treating a cancer.

### CROSS REFERENCE TO RELATED APPLICATIONS

The present application claims priority from U.S. provisional application No. 62/722,726 filed on Aug. 24, 2018 and U.S. provisional application No. 62/804,063 filed on Feb. 11, 2019, both of which are hereby incorporated by reference in their entirety.

### FIELD OF THE DISCLOSURE

The present disclosure relates to methods and compositions for targeting Sortilin receptors and inhibiting vasculogenic mimicry.

### BACKGROUND OF THE DISCLOSURE

According to a recent World Health Organization report, 8.2 million patients died from cancer in 2012 (1). Cancer is therefore a continuously growing health problem in both developing and developed countries. It has also been estimated that the number of annual cancer cases will increase within the next two decades (1). The common general treatments for cancer are surgery, endocrine therapy, chemotherapy, and radiotherapy (2). A recent hope has however been put in the generation of “targeted therapeutics” that home-in on specific molecular defects in cancer cells, promising more effective and less toxic therapies than imprecise chemotherapeutic agents (3).

Currently, when anticancer drugs are administered through classical formulations, it is estimated that ˜95% of the therapeutic agent is taken up by cells within healthy tissues, whereas only ˜2-5% effectively reaches tumors (4). The challenge in any future successful personalized therapeutic approach is therefore to increase selectivity of targeting therapy in part through active transport of anticancer drugs into cancer cell compartments (5-6).

Given its role in ligand internalization and cellular trafficking, Sortilin can be considered as one of the cells' own shuttle system (11). Recent studies demonstrated that Sortilin has a dual role both in endocytosis and in receptor trafficking allowing the sorting of ligands from the cell surface to specific subcellular compartments and the trafficking of pro-neurotrophins such as the neuropeptide neurotensin (NT), proNGF and proBDNF (8, 11-16). Sortilin expression is elevated in several human cancers including breast, prostate, colon, pancreas, skin, and pituitary (17-20). Sortilin has also been reported to be overexpressed in ovarian cancers as compared to healthy ovarian tissue (21, 22).

Vasculogenic mimicry is linked to tumour malignancies, including invasion and metastasis. Vasculogenic mimicry is associated with more aggressive tumor phenotype and a poor 5-year overall survival of cancer patients (24). Vasculogenic mimicry has been described as a process by which cancer cells can establish an alternative blood perfusion pathway through an endothelial cell-free mechanism (25, 29). In addition, vasculogenic mimicry provides also a potential dissemination route for cancer cells (42). In 1999, it was observed that patterned vessel-like channel structures in highly aggressive and metastatic human melanomas in which red blood cells were detected (28). Endothelial cells were not detected in these channels by light microscopy, transmission electron microscopy or immunohistochemical detection of CD34 and CD31 endothelial cell markers.

Vasculogenic mimicry plays a significant role in tumour growth (42) and it has also been characterized in carcinomas of ovary, breast, lung, liver, colorectal, prostate, bladder, kidney, sarcomas and gliomas (43, 29, 44). Survival analyses indicated that patients with vasculogenic mimicry in their tumors had a poor clinical outcome as compared to patients with tumors that do not exhibit vasculogenic mimicry. Meta-analysis studies evaluating the influence of vasculogenic mimicry on cancer patient survival in 15 types of malignant tumours showed that vasculogenic mimicry was associated with a more aggressive tumor phenotype and a poor 5-year overall survival (24, 43). Recently, it has been reported that cancer stem cells (CSCs) and epithelium-to-endothelium transition, a subtype of epithelial-to-mesenchymal transition, can accelerate vasculogenic mimicry by stimulating cancer cell plasticity, remodeling of the extracellular matrix and connecting vasculogenic mimicry channels to host blood vessels (45).

Ovarian cancer was among the first carcinomas in which vasculogenic mimicry was described and correlated with decreased overall patient survival (43). A retrospective study in 120 ovarian carcinoma samples demonstrated that vasculogenic mimicry was involved in 43% of all tested tissues (46). In that same study, CD133 expression, one of the most reliable cell surface markers for cancer stem cells, was found in 47% of ovarian cancer tissues. The presence of both vasculogenic mimicry and CD133-positive expression was associated with advanced tumor stage, high-grade ovarian carcinoma and non-responsiveness to chemotherapy leading to poor prognosis for patients with ovarian cancer. In breast cancer, vasculogenic mimicry has been reported to be highest in triple negative breast cancer (TNBC) specimens (30). In this latter study, CD133+ cells with CSC characteristics were associated with vasculogenic mimicry in TNBC. Furthermore, CD133 expression and vasculogenic mimicry possessed a close relationship in TNBC, as it was suggested that CSC subpopulation inside TNBC-derived MDA-MB-231 cells with a high degree of plasticity triggered vasculogenic mimicry and 3D-tubular structures formation in vitro.

### SUMMARY OF THE DISCLOSURE

Accordingly, a first aspect is a peptide compound having at least 60% sequence identity to a compound chosen from compounds of formula (I), formula (II), formula (III), formula (IV), formula (V), formula (VI), formula (VII), formula (VIII), formula (IX), formula (X), formula (XI) and formula (XII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid;
and wherein at least one protecting group and/or at least one labelling agent is optionally connected to said peptide at an N- and/or C-terminal end,
optionally the peptide compound is cyclic,
for use in inhibiting vasculogenic mimicry and/or treating a cancer.

Another aspect is a peptide compound having at least 80% sequence identity to a compound chosen from compounds of formula (I), formula (II), formula (III), formula (IV), formula (V), formula (VI), formula (Vii), formula (VIII), formula (IX), formula (X), formula (XI) and formula (XII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₈, X₇, X₈, X₉, X₁₀, X₁₁, X₁₂, X₁₃, X₁₄, X₁₅, X₁₈ and X₁₉ are independently chosen from any amino acid;
X₁₈, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid;
and wherein at least one protecting group and/or at least one labelling agent is optionally connected to said peptide at an N- and/or C-terminal end,
optionally the peptide compound is cyclic,
for use in inhibiting vasculogenic mimicry and/or treating a cancer.

In an aspect, there is provided a compound, a peptide compound or derivative thereof that specifically binds to a polypeptide having the amino acid sequence as shown in any one of SEQ ID NOs: 25-50, an analog thereof, or a fragment thereof, for use in inhibiting vasculogenic mimicry.

In an aspect, there is provided a compound, a peptide compound or derivative thereof that targets Sortilin receptor.

In an aspect, there is provided a compound, peptide compound or derivative thereof for use in targeting Sortilin receptor.

In an aspect, there is provided a compound, a peptide compound or derivative thereof that binds at least 2, optionally at least 4 contiguous amino acid residues as shown in any one of SEQ ID NOs: 25-50, an analog thereof, or a fragment thereof.

In a further aspect disclosed herein is a conjugate compound having the formula of A-(B)ₙ,
wherein
   n is 1, 2, 3 or 4;
   A is a peptide compound as defined in the present disclosure, wherein said peptide is optionally protected by a protecting group; and
   B is at least one therapeutic agent, wherein B is connected to A,
   for use in inhibiting vasculogenic mimicry and/or treating a cancer.

In a further aspect disclosed herein is a conjugate compound having the formula of A-(B)ₙ,
wherein
   n is 1, 2, 3 or 4;
   A is a peptide compound as defined in the present disclosure, wherein said peptide compound is optionally protected by a protecting group; and
   B is at least one therapeutic agent, wherein B is connected to A, optionally at a free amine of said peptide compound, at an N-terminal position of said peptide compound, at a free —SH of said peptide compound, or at a free carboxyl of said peptide compound,
   for use in inhibiting vasculogenic mimicry and/or treating a cancer.

A further aspect disclosed herein is a conjugate compound having the formula of A-(B)ₙ,
wherein
   n is 1, 2, 3 or 4;
   A is a peptide compound as defined in the present disclosure, wherein said peptide is optionally protected by a protecting group; and
   B is at least one therapeutic agent, wherein B is connected to A at a free amine of a lysine residue of said peptide compound, optionally via a linker, or at an N-terminal position of said peptide compound, optionally via a linker,
   for use in inhibiting vasculogenic mimicry and/or treating a cancer.

Another aspect disclosed herein is a conjugate compound represented by formula (XXIII):

Acetyl-GVRAK(docetaxel)AGVRN(Nle)FK(docetaxel)SESY Formula (XXIII)
that comprises the peptide compound having SEQ ID NO: 15 wherein each lysine residue has a docetaxel molecule connected thereto.

Another aspect disclosed herein is a conjugate compound represented by formula (XXVIII):

Acetyl-GVRAK(doxorubicin)AGVRN(Nle)FK(doxorubicin)SESY Formula (XXVIII)
that comprises the peptide compound having SEQ ID NO: 15 wherein each lysine residue has a doxorubicin molecule connected thereto.

Another aspect disclosed herein is a conjugate compound represented by formula (LII):

Acetyl-GVRAKAGVRN(Nle)FKSESYC(aldoxorubicin) Formula (LII)
that comprises the peptide compound having SEQ ID NO: 24 wherein cysteine residue has an aldoxorubicin molecule connected thereto, or
that comprises the peptide compound having SEQ ID NO: 15 wherein a cysteine residue is added to C-terminal of said peptide compound, and wherein the cysteine residue has an aldoxorubicin molecule connected thereto.

Another aspect disclosed herein is a conjugate compound chosen from compounds of formula (XVI) and formula (XVII):

Acetyl-GVRAK(curcumin)AGVRN(Nle)FK(curcumin)SESY Formula (XVI)
that comprises the peptide compound having SEQ ID NO: 15 wherein each lysine residue has a curcumin molecule connected thereto; and

Acetyl-YK(curcumin)SLRRK(curcumin)APRWDAPLRDPALRQLL Formula (XVII)
that comprises the peptide compound having SEQ ID NO: 16 wherein each lysine residue has a curcumin molecule connected thereto.

Another aspect disclosed herein is an isolated antibody that specifically binds to a polypeptide having the amino acid sequence as shown in any one of SEQ ID NOs: 25-50, an analog thereof, or a fragment thereof, for use in inhibiting vasculogenic mimicry.

Another aspect disclosed herein is an isolated antibody that targets Sortilin receptor.

Another aspect disclosed herein is an isolated antibody for use in targeting Sortilin receptor. Another aspect disclosed herein is an isolated antibody that binds at least 2, optionally at least 4 contiguous amino acid residues as shown in any one of SEQ ID NOs: 25-50, an analog thereof, or a fragment thereof.

Yet another aspect disclosed herein is a conjugate antibody having the formula of A′-(B)ₙ,
wherein
   n is 1, 2, 3, 4, 5, 6, 7, 8, 9, 10, 11, or 12;
   A′ is an isolated antibody as defined in the present disclosure, wherein said isolated antibody is optionally protected by a protecting group; and
   B is at least one therapeutic agent, wherein B is connected to A′, optionally at a free amine of said isolated antibody, at an N-terminal position of said isolated antibody, at a free —SH of said isolated antibody, or at a free carboxyl of said isolated antibody,
   for use in inhibiting vasculogenic mimicry.
Yet another aspect disclosed herein a conjugate antibody having the formula of A′-(B)ₙ,
wherein
   n is 1, 2, 3, 4, 5, 6, 7, 8, 9, 10, 11, or 12;
   A′ is an isolated antibody as defined in the present disclosure, wherein said isolated antibody is optionally protected by a protecting group; and
   B is at least one therapeutic agent, wherein B is connected to A′ at a free amine of a lysine residue of said isolated antibody, optionally via a linker, or at an N-terminal position of said isolated antibody, optionally via a linker,
   for use in inhibiting vasculogenic mimicry.

In an aspect, there is provided a conjugate antibody that targets Sortilin receptor.

In an aspect, there is provided a process for preparing the conjugate compound or conjugate antibody disclosed in the present disclosure, the process comprising:
reacting a linker together with said at least one therapeutic agent so as to obtain an intermediate;
optionally purifying said intermediate;
reacting said intermediate together with said peptide compound so as to obtain said conjugate compound or conjugate antibody in which said at least one therapeutic agent is connected to said peptide compound or isolated antibody via said linker; and
optionally purifying said conjugate compound or conjugate antibody;
wherein the at least one therapeutic agent is connected to the peptide compound or isolated antibody at a free amine of a lysine residue or at an N-terminal; and wherein the peptide compound comprises 1, 2, 3 or 4 therapeutic agent molecules connected thereto, or the isolated antibody comprises 1, 2, 3, 4, 5, 6, 7, 8, 9, 10, 11, or 12 therapeutic agent molecules connected thereto.

In an aspect, there is provided a method of inhibiting vasculogenic mimicry comprising administering to a subject in need thereof a therapeutically effective amount of at least one compound, isolated antibody, or antibody conjugate as defined herein.

In another aspect, there is provided a method of inhibiting vasculogenic mimicry in cells expressing Sortilin, comprising contacting said cells with at least one compound, isolated antibody, or antibody conjugate as defined herein.

In another aspect, there is provided a method of treating cancer or aggressive cancer, comprising administering to a subject in need thereof a therapeutically effective amount of at least one compound, isolated antibody or antibody conjugate as defined herein.

Also provided is a method of making an isolated antibody, wherein the isolated antibody specifically binds an isolated polypeptide comprising the sequence as shown in any one of SEQ ID NOs: 25-50, an analog thereof, or a fragment thereof, by i) immunizing an animal with an immunogenic form of the isolated polypeptide; ii) screening an expression library; or iii) using phage display.

In an aspect, there is provided a method of inhibiting vasculogenic mimicry in cancerous tissues or cells expressing Sortilin, comprising contacting said cancerous tissues cells with at least one compound, isolated antibody or antibody conjugate defined herein, wherein the inhibiting vasculogenic mimicry comprises decreasing vasculogenic mimicry tube length in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an aspect, there is provided a method of inhibiting vasculogenic mimicry in cancerous tissues or cells expressing Sortilin, comprising contacting said cancerous tissues cells with at least one compound, isolated antibody or antibody conjugate defined herein, wherein the inhibiting vasculogenic mimicry comprises decreasing number of vasculogenic mimicry loops in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an aspect, there is provided a method of inhibiting vasculogenic mimicry in cancerous tissues or cells expressing Sortilin, comprising contacting said cancerous tissues cells with at least one compound, isolated antibody or antibody conjugate defined herein, wherein the inhibiting vasculogenic mimicry comprises decreasing vasculogenic mimicry tube length in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent. An at least 2 fold greater decrease means for example that if vasculogenic mimicry tube length decreases by 10% in cancerous tissues or cells expressing Sortilin treated with the at least one therapeutic agent, then vasculogenic mimicry tube length decreases by at least 20% in cancerous tissues or cells expressing Sortilin treated with the at least one compound, isolated antibody or antibody conjugate described herein.

In an aspect, there is provided a method of inhibiting vasculogenic mimicry in cancerous tissues or cells expressing Sortilin, comprising contacting said cancerous tissues cells with at least one compound, isolated antibody or antibody conjugate defined herein, wherein the inhibiting vasculogenic mimicry comprises decreasing number of vasculogenic mimicry loops in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with at least one therapeutic agent. An at least 2 fold greater decrease means for example that if vasculogenic mimicry loops decreases by 10% in cancerous tissues or cells expressing Sortilin treated with the at least one therapeutic agent, then vasculogenic mimicry loops decreases by at least 20% in cancerous tissues or cells expressing Sortilin treated with at least one compound, isolated antibody or antibody conjugate described herein.

In an aspect, there is provided a method of inhibiting vasculogenic mimicry in cancerous tissues or cells expressing Sortilin, comprising contacting said cancerous tissues cells with at least one compound, isolated antibody or antibody conjugate defined herein, wherein the cells expressing Sortilin are immune cells, optionally macrophages, CD4+, CD8+, B220+, bone marrow-derived cells basophils, eosinophils and cytotoxic T lymphocytes, Natural Killer (NK) cells, T helper type 1 (Th1) cells.

In an aspect, there is provided a use of at least one compound, isolated antibody, or antibody conjugate as defined herein for inhibiting vasculogenic mimicry.

In an aspect, there is provided a use of at least one compound, isolated antibody, or antibody conjugate as defined herein for targeting Sortilin receptor.

In an aspect, there is provided a use of at least one compound, isolated antibody, or antibody conjugate as defined herein for inhibiting vasculogenic mimicry in cancerous tissues or cells expressing Sortilin.

In an aspect, there is provided a use of at least one compound, isolated antibody, or antibody conjugate as defined herein for treatment of cancer or aggressive cancer.

In an aspect, there is provided a use of at least one compound, isolated antibody, or antibody conjugate as defined herein for treatment of cancer or aggressive cancer in cancerous tissues or cells expressing Sortilin.

In an aspect, there is provided a use of at least one compound, isolated antibody, or antibody conjugate as defined herein in the manufacture of a medicament for inhibiting vasculogenic mimicry.

In an aspect, there is provided a use of at least one compound, isolated antibody, or antibody conjugate as defined herein in the manufacture of a medicament for target Sortilin receptor.

In an aspect, there is provided a use of at least one compound, isolated antibody, or antibody conjugate as defined herein in the manufacture of a medicament for inhibiting vasculogenic mimicry in cancerous tissues or cells expressing Sortilin.

In an aspect, there is provided a use of at least one compound, isolated antibody, or antibody conjugate as defined herein in the manufacture of a medicament for treatment of cancer or aggressive cancer.

In an aspect, there is provided a use of at least one compound, isolated antibody, or antibody conjugate as defined herein in the manufacture of a medicament for treatment of cancer or aggressive cancer in cancerous tissues or cells expressing Sortilin.

In an aspect, there is provided a use of at least one compound, isolated antibody or antibody conjugate defined herein, for decreasing vasculogenic mimicry tube length in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an aspect, there is provided a use of at least one compound, isolated antibody or antibody conjugate defined herein, for decreasing number of vasculogenic mimicry loops in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an aspect, there is provided a use of at least one compound, isolated antibody or antibody conjugate defined herein, for decreasing vasculogenic mimicry tube length in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In an aspect, there is provided a use of at least one compound, isolated antibody or antibody conjugate defined herein, for decreasing number of vasculogenic mimicry loops in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In an aspect, there is provided a use of at least one compound, isolated antibody or antibody conjugate defined herein, for use in inhibiting vasculogenic mimicry in cells expressing Sortilin, wherein the cells expressing Sortilin are immune cells, optionally macrophages, CD4+, CD8+, B220+, bone marrow-derived cells basophils, eosinophils and cytotoxic T lymphocytes, Natural Killer (NK) cells, T helper type 1 (Th1) cells.

In an aspect, there is provided a process for preparing the conjugate compound or antibody conjugate disclosed in the present disclosure, the process comprising:
reacting a linker together with said at least one therapeutic agent so as to obtain an intermediate;
optionally purifying said intermediate;
reacting said intermediate together with said peptide compound or isolated antibody so as to obtain said conjugate compound or antibody conjugate in which said at least one therapeutic agent is connected to said peptide compound or isolated antibody via said linker; and
optionally purifying said conjugate compound;
wherein the at least one therapeutic agent is connected to the peptide compound or isolated antibody at a free amine of a lysine residue or at an N-terminal; and wherein the peptide compound comprises 1, 2, 3 or 4 therapeutic agent molecules connected thereto, or the isolated antibody comprises 1, 2, 3, 4, 5, 6, 7, 8, 9, 10, 11, or 12 therapeutic agent molecules connected thereto.

In another aspect, there is provided a method of increasing stability and/or bioavailability of a therapeutic agent, comprising:
obtaining the conjugate compound disclosed herein, wherein said conjugate compound comprises said therapeutic agent, and
administering a therapeutically effective amount of said conjugate compound to a subject in need thereof.

In another aspect, there is provided a method of increasing stability and/or bioavailability of a therapeutic agent, comprising:
conjugating said therapeutic agent with the peptide compound as defined herein to obtain a conjugate compound, and
administering a therapeutically effective amount of said conjugate compound to a subject in need thereof.

In another aspect, there is provided a use of a conjugate compound as defined herein for increasing stability and/or bioavailability of said at least one therapeutic agent.

In another aspect, there is provided a use of at least one compound, isolated antibody, or antibody conjugate as defined herein in the manufacture of a medicament for inhibiting vasculogenic mimicry.

In another aspect, there is provided a use of at least one compound, isolated antibody, or antibody conjugate as defined herein in the manufacture of a medicament for targeting Sortilin receptor.

In another aspect, there is provided herein a method of increasing tolerability of a therapeutic agent, comprising:
conjugating the therapeutic agent with the peptide compound herein disclosed to obtain a conjugate compound, and
administering a therapeutically effective amount of the conjugate compound to a subject in need thereof.

In another aspect, there is provided herein a method of increasing tolerability of a therapeutic agent, comprising:
obtaining a conjugate compound or an antibody conjugate herein disclosed, wherein the conjugate compound or the antibody conjugate comprises the therapeutic agent, a linker, wherein the antibody conjugate targets Sortilin.
administering a therapeutically effective amount of the conjugate compound or the antibody conjugate to a subject in need thereof.

For example, there is provided a use of a conjugate compound or an antibody conjugate herein disclosed, for increasing tolerability of a therapeutic agent.

In a further aspect, there is provided a liposome, graphene, nanotube or nanoparticle comprising at least one compound, isolated antibody or antibody conjugate as defined herein for use in inhibiting vasculogenic mimicry.

In a further aspect, there is provided a liposome, graphene, nanotube or nanoparticle comprising at least one compound, isolated antibody or antibody conjugate as defined herein for use in targeting Sortilin receptor.

In a yet another aspect, there is provided a liposome, graphene, nanotube or nanoparticle coated with at least one compound, isolated antibody or antibody conjugate as defined herein for use in inhibiting vasculogenic mimicry.

In a yet another aspect, there is provided a liposome, graphene, nanotube or nanoparticle coated with at least one compound, isolated antibody or antibody conjugate as defined herein for use in targeting Sortilin receptor.

### BRIEF DESCRIPTION OF THE FIGURES

Further features and advantages of the disclosure will become more readily apparent from the following description of specific embodiments as illustrated by way of examples in the appended schemes and figures wherein:

FIG. 1 is a prior art representation depicting a graph showing percentage of human cancers presenting vasculogenic mimicry (VM) from ref 24 (n=3062 clinical cases). vasculogenic mimicry formation expression was associated with advanced tumor stage, high-grade carcinoma and nonresponse to chemotherapy. Vasculogenic mimicry was also associated with shorter overall survival time in various cancers. Abbreviations: BDMT: bi-directional differentiated malignant tumour; HCC: hepatocellular carcinomas; NSCLC: non-small cell lung cancer; OLSCC: oral/laryngeal squamous cell carcinoma.

FIG. 2 is a prior art representation depicting a graph showing percentage of breast cancers with vasculogenic mimicry (VM). In breast cancers, the presence of vasculogenic mimicry is the highest in Triple Negative (TN) breast cancer patients when compared to luminal or HER2+ positive breast cancers. Vasculogenic mimicry could represent an important survival mechanism contributing to failure of current anti-angiogenesis therapies to fully effect tumor eradication (30).

FIG. 3 is a prior art representation showing in vitro 3D-reconstruction of X-ray microtomography of vasculogenic mimicry using SKOV3 ovarian cancer cells (from ref. 31). Reconstructed view of the landscape of a 4 day 3D-culture of ovarian cancer cells on Matrigel showing elevated structures with tubular-like appearances are clearly visible (panel a). The structures within the white rectangle are shown in higher magnification in panel b and c, with the arrowhead denoting a seemingly tubular structure projecting above the flat cell aggregates. A cross-section of this structure is shown to demonstrate an air-filled space with an estimated diameter of 50 μm (panel c).

FIG. 4 shows a prior art confocal microscopy of vasculogenic mimicry tubular structure using SKOV3 expressing the green fluorescent protein (GFP) (from ref. 31). Panel a: A confocal microscopy Z-stack reconstruction demonstrating the presence of a cell containing tubular structure. Z-Stack shows a continuous upper monolayer [1], with central walled structures with a hollow center [2] and a continuous lower monolayer [3]. Panel b: Computer-generated cross-section clearly showing lumen-containing tubular structures.

FIG. 5 shows formation of 3D-tubular structures by ES-2 ovarian cancer cells. Tube-like structures in ES-2 ovarian cancer cells were rapidly formed and observed within 4 h after seeding on Matrigel.

FIGS. 6A, 6B, 6C and 6D show detection of Sortilin in 3D-tubular structures of ES-2 ovarian cancer cells. More particularly, FIG. 6A shows ES-2 ovarian cancers cells seeded on Matrigel. After 12 hrs, Sortilin was detected in 3D-tubular structures by confocal microscopy using a rabbit anti-Sortilin antibody. FIG. 6B shows a control with only the secondary anti-rabbit antibody. Overall results indicate that Sortilin detection in (FIG. 6A) is specific. FIG. 6C and FIG. 6D show the DAPI staining of ES2 cancer cell nuclei in the 3D-tubular structures under the conditions used for the anti-SORT1 and secondary antibody detections.

FIGS. 7A and 7B show the effect of Sortilin gene silencing on vasculogenic mimicry formation. ES-2 ovarian cancer cells were transfected with either scrambled siRNA (siScrambled) or specific SORT1 siRNA (siSORT1) and seeded on Matrigel. After 12 hrs, 3D-tubulare structures were observed in ES-2 cancer cells transfected with scrambled siRNA. ES-2 transfected with specific SORT1 siRNA do not form 3D-tubular structures. Quantitation of total tube length and total number of loops by Wimasis Image Analysis software showed that these structures were inhibited by more than 90% when Sortilin expression was reduced by siSORT1 (n=4).

FIGS. 8A and 8B show the effect of Doxorubicin-conjugate compound (DoxKA) on vascukogenic mimicry. More particularly, FIG. 8A shows ES2 ovarian cancer cells were seeded on Matrigel in the presence of increasing concentrations of either DoxKA, Doxorubicin or Doxil (Doxorubicin encapsulated in a liposome). After 12 hrs, 3D-tubular structures were inhibited by DoxKA and not by Doxorubicin or Doxil alone. FIG. 8B shows quantitation of total number of loops and total tube length by Wimasis Image Analysis software, and that these structures were inhibited at low nM concentrations of DoxKA.

FIG. 9 shows the effect of Aldoxorubicin-conjugate compound (AldoxKA) on vasculogenic mimicry. The Doxorubicin derivative Aldoxorubicin was conjugated on Katana peptide with a linker sensitive to acidic pH. ES-2 ovarian cancer cells were seeded on Matrigel in the presence of increasing concentrations of AldoxKA. After 12 hrs, 3D-tubulare structures were inhibited by AldoxKA and not by Doxorubicin alone (see FIG. 8).

FIGS. 10A and 10B show the effect of Docetaxel-conjugate compound (DoceKA) on vasculogenic mimicry. ES-2 ovarian cancer cells were seeded on Matrigel in the presence of either Docetaxel or DoceKA. After 12 hrs, DoceKA showed stronger inhibition of 3Dtubulare structures than Docetaxel alone at an equivalent concentration of Docetaxel (600 pM; FIG. 10A). Quantitation of total number of loops and total tube length showed that these structures were inhibited at low nM concentrations of DoceKA (FIG. 10B).

FIG. 11 shows the effect of Curcumin-conjugate compound (CurKA) on vasculogenic mimicry. ES-2 ovarian cancer cells were seeded on Matrigel in the presence of either Curcumin or CurKA. After 12 hrs, CurKA showed stronger inhibition of 3D-tubulare structures than Curcumin alone at an equivalent concentration of Curcumin. Total number of loops and total tube length showed that these structures were inhibited at nM concentrations of CurKA.

FIG. 12 shows the inhibition of vasculogenic mimicry by Anti-Sortilin (Anti-SORT1) mAb. ES-2 ovarian cancer cells were seeded on Matrigel in the presence of the vehicle (Control), mouse IgG (12 nM) or anti-SORT1 mAb (12 nM). Images were taken after 4 and 12 hrs. Anti-SORT1 mAb inhibits the formation of 3D-tubular structures and strongly affects total tube length and number of loops (n=2).

FIGS. 13A, 13B, 13C and 13D represent a series of graphs showing the binding and internalization of anti-SORT1 mAb to ES-2 ovarian cancer cells. FIG. 13A shows the labeling of anti-Sortilin antibody was performed with Alexa Fluor 488 protein labeling kit from Invitrogen. Human ES-2 ovarian cancer cells were incubated at 4° C. with anti-Sortilin-Alexa488 (1 μg/ml) for 30 minutes followed or not by trypsinization to assess the binding at the cell surface. Results clearly demonstrate that the major portion of the fluorescence signal was caused by the binding to the Sortilin receptor at the cell surface since trypsinization reduced the fluorescence levels by more than 90%. FIG. 13B shows the binding of the anti-Sortilin-Alexa488 (1 μg/ml, 30 minutes) at the cell surface of the human ES-ovarian cancer cells was performed in the presence of Sortilin ligands (neurotensin (NT) and progranulin (PGRN)) and in the presence of Katana peptides (KBP106 and KBP201). FIG. 13C shows anti-Sortilin antibody internalization into ES-2 cancer cells. In this experiment, binding of the anti-Sortilin-Alexa488 antibody (1 μg/ml, 30 minutes) on human ES-2 ovarian cancer cells was first performed at 4° C., then the cells were washed to remove unbound fluorescent antibody and cells were incubated at 37° C. for 1 or 2 hrs and then trypsinized. Fluorescence associated with internalized fluorescent anti-Sortilin-Alexa488 was then quantified by flow cytometry. Results indicate that about 50% of anti-Sortilin-Alexa488 that was first bound on the cell surface was internalized within 2 hrs. FIG. 13D shows the internalization of anti-Sortilin-Alexa488 was measured as described in FIG. 13C with increasing concentrations. Results demonstrate that the internalization of the anti-Sortilin-Alexa488 fluorescent conjugate increased as a function of fluorescent conjugate concentration and was saturable.

FIG. 14 shows a schematic diagram of different regions of Sortilin and the regions where the anti-Sortilin antibodies used in this disclosure were generated against.

FIG. 15 shows inhibition of vasculogenic mimicry of ovarian cancer cells by anti-Sortilin antibodies. Inhibition of vasculogenic mimicry by Anti-Sortilin (Anti-SORT1) mAb. ES-2 ovarian cancer cells were seeded on Matrigel in the presence of the vehicle (Control), mouse IgG (12 nM) or anti-SORT1 mAb #1 (12 nM), or anti-SORT1 mAb #2 (12 nM). Images were taken at 0 h and after 12 hrs of antibody treatment. Results show that anti-SORT1 mAb inhibits the formation of 3D-tubular structures and strongly affects total tube length and number of loops (n=2).

FIGS. 16A and 16B show effects of anti-Sortilin antibodies on vasculogenic mimicry of ES-2 ovarian cancer cells. More particularly, FIG. 16A shows anti-Sortilin mAb #1 and #2 decrease the total number of vasculogenic mimicry loops. FIG. 16B shows anti-Sortilin mAb #1 and #2 decrease the total vasculogenic mimicry tube length. The anti-Sortilin mAb #2 generated against the Sortilin extracellular amino acid sequence (300-422) strongly inhibits vasculogenic mimicry.

FIGS. 17A, 17B, 17C and 17D show histological characterization of Sortilin expression in normal and breast cancer cells and Alexa488-labelled KA-peptide uptake assessment in MDA-MB-231 cells. More particularly, FIG. 17A is an immunohistochemical technique showing the absence of Sortilin expression in normal adjacent tissue and its overexpression in stage IIIC infiltrating duct carcinoma (IDC) and lymph node metastasis carcinoma (LNMC). Sortilin intensity of staining was evaluated using the IS method. FIG. 17B demonstrates distribution of Sortilin in normal tissues versus IDC and LNMC breast tumours. FIG. 17C illustrates gene silencing of Sortilin was performed as described in the Methods section and validated by western blotting using Anti-SORT1 antibody. Uptake of 200 nM Alexa488-labeled KA-peptide was next performed in control (siScrambled) or Sortilin-deficient (siSORT1) MDA-MB-231 cancer cells. FIG. 17D depicts the uptake of 200 nm Alexa488-labeled KA-peptide was also evaluated in MDA-MB-231 cells in the absence (white bar) or presence (black bars) of excess unlabeled KA-peptide (50 μM), Neurotensin (10 μM) or Progranulin (1 nM).

FIG. 18A illustrates the synthesis of Docetaxel-Katana peptide conjugate. Diisopropylethylamine (DIEA; 0.21 ml, 1.2 mmol) was added dropwise to a suspension of Docetaxel (0.81 g, 1.0 mmol) and succinic anhydride (105 mg, 1.05 mmol) in DMSO (5 m) under stirring. The mixture was stirred at room temperature and monitored by UPLC-MS. After 2 h, the reaction was completed. The solvent was removed, and the resulting residue was dissolved in dichloromethane and loaded on Biotage silica column for purification. DoceSuOH was obtained as a white powder after lyophilization, UPLC-MS purity >95%. DIEA (0.234 mmol) was added dropwise to a solution of DoceSuOH (213 mg, 0.234 mmol) and TBTU (75 mg, 0.234 mmol) in DMSO (3-4 ml) in order to preactivate the DoceSuOH. The completion of preactivation was monitored by UPLC-MS, then a solution of KA-peptide (120 mg, 0.062 mmol) in dimethylsulfoxide (0.2 m) was added. The mixture was stirred at room temperature. The reaction was monitored by UPLC-MS until completion. The reaction mixture was purified using 3ORPC resin column and an AKTA purifier system (10% to 80% ACN) to give KA(SuDoce)2 or DoceKA as white powder after lyophilisation. FIG. 18B illustrates that the purity of the conjugate evaluated by UPLC-MS purity was greater than 95%.

FIG. 19 depicts the in vitro anticancer properties of DoceKA against MDA-MB-231 breast cancer cells as determined using the [3H]-Thymidine Incorporation assay. Incorporated [3H]-Thymidine was plotted for each drug concentration. IC50 values (nM) were calculated using the GraphPad Prism software. The IC50 value for DoceKA was 0.38 nM and the IC50 value for Docetaxel was 0.68 nM. FIG. 19 is a set of graphs depicting the cell-cycle distribution of MDA-MB231 cells determined based on cellular DNA content using flow cytometry after 24 hours of treatment with 2 μM Docetaxel or 1 μM DoceKA.

FIGS. 20A and 20B illustrate the impact of Sortilin gene silencing on the anti-migratory potential of DoceKA. More particularly, FIG. 20A depicts the effect of Docetaxel on MDA-MB-231 cell migration and FIG. 20B depicts the effect of DoceKA on MDA-MB-231 cell migration after SiRNA-mediated gene silencing of Sortilin. Transfected cells were pre-incubated for 2 hours with DoceKA (1 μM) or unconjugated Docetaxel (2 μM). Cells were then harvested and their migratory potential was assessed in real-time using the Xcelligence instrument.

FIGS. 21A, 21B and 21C illustrates cell death induction of Docetaxel and DoceKA conjugate in MDA-MB-231 cells. MDA-MB-231 cells treated for 5 hours with control (vehicle) or with increasing concentrations of Docetaxel or DoceKA. Cells were then harvested and the rate of apoptosis was determined following staining with Annexin V-FITC and propidium iodide (PI). Cells were analyzed by flow cytometry. More particularly, FIG. 21A depicts MDA-MB-231 cells were treated with control (vehicle), 10 μM Docetaxel or 5 μM DoceKA for 24 hours and cellular morphology was examined under a light microscope. FIG. 21B is a line graph showing percentages of apoptotic cell death based on various concentrations of DoceKA and Docetaxel. FIG. 21C depicts that DoceKA uptake was competed by an excess of free KA-peptide (50 μM) or Sortilin ligands, Neurotensin (10 μM) or Progranulin (1 nM) in MDA-MB-231 cells. Following 5 hours of incubation, cells were preceded to Annexin V-FITC/PI staining as shown in FIG. 21A.

FIGS. 22A and 22B illustrate the molecular mechanism of cell death induction by DoceKA in MDA-MB-231 cells. FIG. 22A illustrates the immunoblotting experiment showing the expression level of IL-6, Survivin, Bcl-xi and mutated p53 in MDA-MB-231 cell after treatment with Docetaxel and DoceKA. GAPDH was used as a control. FIG. 22B data are representative of three independent experiments and the bar graphs show the densitometric quantification of IL-6, Survivin, Bcl-xL and p53 expression as compared to GAPDH. The mean value of the control group was set to be 1.0.

FIGS. 23A and 23B depict the DoceKA impact on tubulin polymerization. More particularly, FIG. 23A illustrates cells treated with vehicle (DMSO), 2 μM Docetaxel or 1 μM DoceKA for 24 hours, fixed and immunostained with anti-a-tubulin antibody and imaged using confocal microscopy. DNA was stained with DAPI, and cells were visualized using confocal microscopy Representative cells from each condition are displayed. FIG. 23B illustrates that in a fluorescence-based polymerization assay, the effect of Docetaxel (2 μM) and DoceKA (1 μM) on polymerization of purified tubulin was examined in vitro. Paclitaxel (2 μM) and Vinblastine (2 μM) were added as controls for tubulin-polymerizing and tubulin-depolymerizing agents, respectively. Tubulin assembly into microtubules was determined by an increase in fluorescence emission (Ex. 340-360 nm±20 nm; Em. 410-460 nm±20 nm).

FIG. 24A depicts the residual tumor burden after no treatment (vehicle), treatment with Docetaxel (Docetaxel), or treatment with a Docetaxel conjugate (DoceKA). FIG. 24B illustrates the size of the tumor after no treatment, treatment with Docetaxel, or DoceKA 14 days after treatment, and 74 days after treatment. FIG. 24C illustrates the residual tumor burden 74 days after treatment with Docetaxel or DoceKA.

FIGS. 25A and 25B depict the effect of DoceKA on MDA-MB231 TNBC xenograft model. More particularly, FIG. 25A depicts the tumor volume in mice after treatment with either Docetaxel or DoceKA. Those treated with Docetaxel received 3 treatments at 15 mg/kg/week (MTD). Those treated with DoceKA received 5 treatments at an equivalent dose of Docetaxel. FIG. 25B depicts the weight of mice at various points after treatment with Docetaxel or DoceKA. Those treated with Docetaxel received 3 treatments at 15 mg/kg/week (MTD). Those treated with DoceKA received 5 treatments at an equivalent dose of Docetaxel. One mouse was sacrificed at day 15 because of toxicity (body weight: −25%).

FIGS. 26A and 26B depicts the DoceKA dose-response on MDA-MB231 TNBC model. More particularly, FIG. 26A illustrates the effect on the tumor volume after treatment with various dosages of DoceKA. FIG. 26B depicts the effect on tumour volume progression at different dosages of DoceKA at day 15 after treatment.

FIGS. 27A, 27B and 27C demonstrate the absence of effect by DoceKA on blood cells in nude mice. FIG. 27A illustrates the effect on lymphocytes after 3 treatments with different concentrations of Docetaxel or DoceKA. FIG. 27B illustrates the effect on platelets after 3 treatments with different concentrations of Docetaxel or DoceKA. FIG. 27C illustrates the effect on neutrophils after 3 treatments with different concentrations of Docetaxel or DoceKA. IP means intraperitoneal administration, while IV means intravenous delivery.

FIG. 28 depicts the preliminary toxicity data on DoceKA, specifically, the neutrophils count (g/L) after DoceKA treatments at different dosages of Docetaxel or DoceKA. Four days after a single injection at the maximum tolerable dose, Docetaxel induced a drastic reduction in neutrophils count; whereas, even after six DoceKA injections, neutrophils count remained within normal limits.

FIG. 29A illustrates the plasma concentration for DoceKA and released Docetaxel (BDL) at progressive time intervals. FIG. 29B illustrates the PK in CD-1 mice. DoceKA (20 mg/kg) was administered intravenously to mice. Plasma was collected at different time points. DoceKA and released Docetaxel were extracted with acetonitrile. DoceKA and released Docetaxel, were quantified by UPLC/MS using Paclitaxel as the internal standard: 2 μl and 10 μl of supematants were injected for DoceKA and Docetaxel directly without lyophilization. Concentration of released Docetaxel was below the limit of quantification (LOQ) for Docetaxel at 0.083, 0.25 and 4 hours. Reported Cmax for Docetaxel dosed at 20 mg/kg is about 20 μg/ml or 25 μM.

FIG. 30 illustrates Sortilin expression in human ovarian cancer and normal tissues by immunohistochemistry. The Sortilin expression increases progressively from normal cells, to benign cells, to borderline cells, to malignant cells, to metastatic cells.

FIG. 31A illustrates Sortilin expression in human ovarian cancer and normal tissues as measured using immunohistochemistry. FIG. 31B depicts Sortilin expression for normal, benign, low-grade serous carcinoma, high-grade serous carcinoma, clear cell carcinoma, mucinous carcinoma, endometrioid carcinoma, transitional cell carcinoma, borderline, metastatic cells, and germ cell and other non-epithelial cells.

FIG. 32 illustrates human ovarian cancer Sortilin gene expression in cDNA tissue microarray (OrGene). Specifically, it illustrates the Sortilin gene expression in healthy tissue and ovarian tumors grades I to IV using qPCR quantification.

FIG. 33A is a series of immunohistochemistry images of normal breast tissue and breast cancer tissue exposed to anti-SORT1 mAB. FIG. 33B depicts Sortilin expression in normal tissue, infiltrating ductal carcinoma, and lymph node metastatic carcinoma tissue.

FIG. 34 depicts immunohistochemistry images of the immunohistochemistry in three different patients with infiltrating ductal carcinoma (Stage IIIC), associated lymph node metastasis, and adjacent normal tissue. The IHC samples are stained with anti-SORT1 mAB.

FIG. 35A depicts images of immunohistochemistry of Sortilin in melanomas. Specifically, the images show Sortilin in normal tissue, and melanoma II, II, and IV stage tumors. FIG. 35B depicts the Sortilin expression in normal tissue and stages I to IV melanoma tumor tissues in a bar graph.

FIG. 36 is a series of images showing Sortilin in normal tissue, and melanoma II, II, and IV stage tumors.

FIGS. 37A and 37B depict the immunohistochemistry of Sortilin in uterine cancers. More particularly, FIG. 37A consists of images of Sortilin in normal, endometrial cancer, and cervical cancer tissues. FIG. 37B is a graphical representation of the Sortilin expression in normal, endometrial, and cervical cancer tissue.

FIGS. 38A and 38B depict the immunohistochemistry of Sortilin in lung cancers. More particularly, FIG. 38A consists of images of Sortilin in normal lung tissue and cancerous lung tissues. FIG. 38B is a graphical representation of the Sortilin expression in normal and cancerous lung tissues.

FIG. 39 is a series of western blot images illustrating Sortilin expression in several different cancer cell lines (ovary, breast, brain, and other cancers).

FIGS. 40A and 40B depict the confocal microscopy imaging of 3D tubular structure in OVCAR-3 ovarian cancer cells. Sortilin was detected using a rabbit anti-Sortilin antibody. More particularly, FIG. 40A is three paneled with the first image depicting the staining of Sortilin present in the cell, the second image depicting Hoechst staining of the nucleus of the cell and the final image depicting the two previous images merged. FIG. 40B is three paneled with the first image depicting the staining of a control antibody present in the cell, the second image depicting Hoechst staining of the nucleus of the cell and the final image depicting the two previous images merged. These results indicate that Sortilin positive cells contribute to vasculogenic mimicry in vitro.

FIGS. 41A, 41B and 41C illustrates the inhibition of vasculogenic mimicry by Docetaxel-conjugated compound (DoceKA) in ES-2 ovarian cancer cells. More particularly, FIG. 41A illustrates the total loops present when different concentrations of Docetaxel or DoceKa are present. FIG. 41B illustrates the total tube length present when different concentrations of Docetaxel or DoceKa are present. FIG. 41C illustrates the percent of branching points present when different concentrations of Docetaxel or DoceKa are present. These figures illustrate that a low pM concentration of DoceKA inhibits the Vasculogenic Mimicry of ES-2 ovarian cancer cells.

FIGS. 42A, 42B, 42C, 42D, 42E and 42F depicts the effect of Sortilin gene silencing on in vitro vasculogenic mimicry in TNBC-derived MDA-MB231 cells. More particularly, FIG. 42A illustrates MDA-MB231 cells transiently transfected with scrambled siRNA (siScrambled) at 0 hours. FIG. 42B illustrates MDA-MB231 cells transiently transfected with specific sortilin siRNA (siSortilin) at 0 hours. FIG. 42C illustrates MDA-MB231 cells transiently transfected with scrambled siRNA (siScrambled) at 24 hours. FIG. 42D illustrates MDA-MB231 cells were transiently transfected with specific sortilin siRNA (siSortilin) at 24 hours. FIG. 42E is a bar graph depicting the percentage of total loops of the siScrambled and siSortilin cells over 24 hours. FIG. 42F is a bar graph depicting the percent of mean loop area of the siScrambled cells and siSortilin cells over 24 hours.

FIGS. 43A and 43B depict inhibition of MDA-MB231 vasculogenic mimicry by DoceKA. More particularly, FIG. 43A illustrates the effect of different concentrations of Docetaxel (top panel) and DoceKa (bottom panel) administered to the cells. FIG. 43B is a graph illustrating the total number of loops present in the cell when exposed to different concentrations of Docetaxel and DoceKA.

FIG. 44A illustrates the vasculogenic mimicry of ES-2 ovarian cancer cells at 0, 2, 6, 12 and 24 hours. FIG. 44B depicts the gene expression of Sortilin (SORT1), CD133 and MMP9 at 0, 2, 6, 12, and 24 hours. CD133 is one of the most commonly used markers for isolation of cancer stem cells (CSC) population from tumors. CD133+ cancer cells (CSC) were positively associated with vasculogenic mimicry formation, local regional recurrence and distant metastasis.

FIG. 45A illustrates the vasculogenic mimicry of MDA-MB-231 TNBC cells at 0, 2, 6, 12 and 24 hours. FIG. 45B depicts the gene expression of Sortilin (SORT1), CD133 and MMP9 at 0, 2, 6, 12, and 24 hours.

FIGS. 46A and 46B depict the inhibition of vasculogenic mimicry in ES-2 ovarian cancer cells by anti-Sortilin antibodies. More particularity, FIG. 46A depicts images, taken after 12 hours, of the ES-2 ovarian cancer cells in the presence of rabbit Ig, anti-SORT1 rabbit pAb, mouse IgG, and anti-SORT1 mouse mAb. FIG. 46B illustrates the total number of loops present in the cells exposed to Rabbit IgG, anti-Sortilin Rabbit pAb, Mouse IgG and anti-Sortilin mAb at 12 hours. Anti-SORT1 mAb inhibits the formation of 3D-tubular structures and strongly affects total tube length and number of loops.

FIG. 47 depicts paneled images of ES-2 cells ovarian cancer cells in the presence of a control or anti-SORT1 antibody in various concentrations, at 0 and 12 hours.

FIGS. 48A and 48B illustrate the effect of anti-Sortilin on vascukogenic mimicry in ES-2 ovarian cancer cells. FIG. 48A depicts the total number of loops present in the cells at different concentrations of anti-SORT1 antibody. FIG. 48B illustrates the percentage proliferation of the cells exposed to different concentrations of anti-SORT1 antibody as compared to a control not exposed to anti-Sortilin.

FIG. 49 is a paneled image illustrating ES-2 ovarian cancer cells exposed to various concentrations of the Katana peptide (KBP106) alone at 0 and 24 hours of exposure. Under the same experimental conditions as used for the Katana conjugates, the peptide (KBP106) has no significant effect on vascukogenic mimicry up to 50 μM.

FIG. 50 is a paneled image illustrating ES-2 ovarian cancer cells exposed to the KBP106 peptide and Doxorubicin conjugate (KBB106). FIG. 50 depicts images of the ES-2 ovarian cancer cells exposed to various concentrations of the KBP106 peptide and KBB106 at 0 and 24 hours of exposure. An addition of an excess of KBP106 was shown to reverse vascukogenic mimicry inhibition caused by KBB106. Despite KBP106 having no effect on vascukogenic mimicry, the addition of KBP106 to KBB106 prevents vascukogenic mimicry inhibition by KBB106, suggesting that KBP106, by binding to Sortilin, prevents the interaction of KBB106 with the receptor.

FIG. 51 depicts images of ES-2 ovarian cancer cells exposed to Sortilin ligands, neurotensin and progranulin at 0 and 12 hours of exposure to each ligand. This figure demonstrates that Sortilin ligands, neurotensin and progranulin, do not effect vasculogenic mimicry.

FIG. 52 is a paneled image illustrating ES-2 ovarian cancer cells exposed to the KBB106 conjugate and either the neurotensin ligand or the progranulin ligand at 0 and 12 hours of exposure. These images demonstrate that the addition of the Sortilin ligands, neurotensin and progranulin reverses vasculogenic mimicry inhibition by the KBB106 conjugate.

FIGS. 53A, 53B, 53C, 53D and 53E consist of images of tissue sections from ES-2 xenograft tumors in nude mice. They are examples of single labelings at 40×. FIG. 53A illustrates the labelling of CASPASE-3; FIG. 53B illustrates the labelling of PAS; FIG. 53C illustrates the labelling of Ki-67; FIG. 5D illustrates the labelling of Mouse CD31; and FIG. 53E illustrates the labelling of CD133.

FIG. 54 shows examples of vasculogenic mimicry identification by immunohistochemistry. PAS-positive and CD31-positive indicate normal mouse vasculature, identified in the image as “blood vessels”. PAS-positive and CD31-negative indicate vasculogenic mimicry, identified in the image as “VM”.

FIGS. 55A, 55B and 55C consist of panels of images depicting ES-2 tumor tissue stained for mouse CD31-PAS (FIG. 55A), Sortilin-PAS (FIG. 55B), and CD133-PAS (FIG. 55C), respectively. All figures depict four progressively enlarged images of the stained tissue. In FIG. 55A, blood vessels are identified as “blood vessels” and vasculogenic mimicry is identified by “VM”.

FIGS. 56A, 56B and 56C consist of panels of images depicting ES-2 tumor tissue stained for mouse CD31-PAS (FIG. 56A), Sortilin-PAS (FIG. 56B), and CD133-PAS (FIG. 56C), respectively. All figures depict four progressively enlarged images of the stained tissue. In FIG. 56A, blood vessels are identified by the word “blood vessels”, while vasculogenic mimicry is identified by the word “VM”.

FIGS. 57A, 57B and 57C consist of images depicting ES-2 tumor tissue stained for mouse CD31-PAS (FIG. 57A), Sortilin-PAS (FIG. 57B), and CD133-PAS (FIG. 57C). These figures demonstrate Sortilin and CD133 immunohistochemical detection in vasculogenic mimicry.

FIG. 58 is an image of a western blot analysis under non-denaturing conditions that illustrates the presence of Sortilin in various triple negative breast cancer (TNBC) cell lines (MDA-MB 231, MDA-MB 488, MDA-MB 157, DU4475, HCC70, BT-20) as well as ES-2 ovarian cell line.

FIGS. 59A and 59B illustrate another example of BT-20 TNBC cancer cells that can form 3D-tubular structures. More particularly, FIG. 59A depicts 20, 000 BT-20 TNBC cancer cells before developing vasculogenic mimicry structure, while FIG. 59B depicts the same cells after the formation of vasculogenic mimicry structures.

### DETAILED DESCRIPTION OF THE DISCLOSURE

The term “peptide compounds” or “Katana peptides”, “Katana Biopharma Peptide” or “KBP” as used herein refers, for example, to peptides derived from bacterial proteins or from ligands of receptors that target receptors expressed on cancer cells including multidrug resistant cancer cells. For example, the peptide compounds can be derived from bacterial proteins involved in cell penetration or from sortilin ligands, for example progranulin and neurotensin. For example, the peptide compounds can be cyclic. In certain embodiments, peptide compounds are connected (for example via a covalent bond, an atom or a linker) to at least one therapeutic agent (such as an anticancer agent or a phytochemical), thereby forming a conjugate compound that can be used, for example, for treatment of a cancer or aggressive cancer. In certain other embodiments, peptide compounds can be used at the surface of liposomes. For example, the peptide compounds can be used for coating liposomes, graphene, nanotubes or nanoparticles that can be loaded with at least one therapeutic agent (such as an anticancer agent or phytochemical, or genes or siRNA).

The term “Katana Biopharma Peptide Family 1 peptide compounds” or “KBP Family 1 peptide compounds” refers to peptide compounds derived from bacterial cell penetrant proteins. For example, KBP Family 1 peptide compounds can be derived from a protein having an amino acid sequence of IKLSGGVQAKAGVINMDKSESM (SEQ ID NO: 5). Non limiting examples of KBP Family 1 peptide compounds are shown below:

Amino acid sequences

| | |
|---|---|
| KBP-101 | IKLSGGVQAKAGVINMDKSESM-Formula (V) (represented by SEQ ID NO: 5) |
| KBP-102 | Succinyl-IKLSGGVQAKAGVINMFKSESY-Formula (XXXVI) (comprises SEQ ID NO: 6 |
| | wherein a succinyl group is attached at the N-terminal end) |
| KBP-103 | IKLSGGVQAKAGVINMFKSESYK(Biotin)-Formula (XXXVII) (comprises SEQ ID NO: 7 |
| | wherein a biotin molecule is connected thereto at the C-terminal end) |
| KBP-104 | GVQAKAGVINMFKSESY-Formula (VIII) (represented by SEQ ID NO: 8) |
| KBP-105 | Acetyl-GVRAKAGVRNMFKSESY-Formula (XXXVIII) (represented by SEQ ID NO: 14) |
| KBP-106 | Acetyl-GVRAKAGVRN(Nle)FKSESY-Formula (XXXIX) (represented by SEQ ID NO: 15) |

As used herein, the peptide compound KBP-101 is represented by the amino acid sequence of IKLSGGVQAKAGVINMDKSESM (SEQ ID NO: 5).

As used herein, the peptide compound KBP-102 is represented by the amino acid sequence of Succinyl-IKLSGGVQAKAGVINMFKSESY that comprises the peptide sequence of SEQ ID NO: 6 wherein a succinyl group is attached thereto at the N-terminal end.

As used herein, the peptide compound KBP-103 is represented by the amino acid sequence of IKLSGGVQAKAGVINMFKSESYK(Biotin) that comprises the peptide sequence of SEQ ID NO: 7 wherein a biotin molecule is connected thereto at the C-terminal end.

As used herein, the peptide compound KBP-104 is represented by the amino acid sequence of GVQAKAGVINMFKSESY (SEQ ID NO: 8).

As used herein, the peptide compound KBP-105 is represented by the amino acid sequence of Acetyl-GVRAKAGVRNMFKSESY (SEQ ID NO: 14).

As used herein, the peptide compound KBP-106 is represented by the amino acid sequence of Acetyl-GVRAKAGVRN(Nle)FKSESY (SEQ ID NO: 15).

The term “Katana Biopharma Peptide Family 2 peptide compounds” or “KBP Family 2 peptide compounds” refers to peptides derived from sortilin ligands, progranulin and neurotensin. For example, peptides can be derived from human, rat or mouse progranulin. For example, KBP Family 2 peptide compounds can be derived from human progranulin, for example having the amino acid sequence KCLRREAPRWDAPLRDPALRQLL (SEQ ID NO: 19), from rat progranulin, for example having the amino acid sequence KCLRKKTPRWDILLRDPAPRPLL (SEQ ID NO: 20), from mouse progranulin, for example having the amino acid sequence KCLRKKIPRWDMFLRDPVPRPLL (SEQ ID NO: 21), or from neurotensin, for example having an amino acid sequence XLYENKPRRPYIL (SEQ ID NO: 22). Non limiting examples of KBP Family 2 peptide compounds are shown below:

Amino acid sequences

| | |
|---|---|
| KBP-201 | Acetyl-YKSLRRKAPRWDAPLRDPALRQLL-Formula (XXXX) (represented by SEQ |
| | ID NO: 16) |
| KBP-202 | Acetyl-YKSLRRKAPRWDAYLRDPALRQLL-Formula (XXXXI) (represented by |
| | SEQ ID NO: 17) |
| KBP-203 | Acetyl-YKSLRRKAPRWDAYLRDPALRPLL-Formula (XXXXII) (represented by |
| | SEQ ID NO: 18) |

As used herein, the peptide compound KBP-201 is represented by the amino acid sequence of Acetyl-YKSLRRKAPRWDAPLRDPALRQLL (SEQ ID NO: 16).

As used herein, the peptide compound KBP-202 is represented by the amino acid sequence of Acetyl-YKSLRRKAPRWDAYLRDPALRQLL (SEQ ID NO: 17).

As used herein, the peptide compound KBP-203 is represented by the amino acid sequence of Acetyl-YKSLRRKAPRWDAYLRDPALRPLL (SEQ ID NO: 18).

The term “Sortilin” or “Sortilin receptor” as used herein refers to a neuronal type-1 membrane glycoprotein, encoded by the SORT1 gene, belonging to the Vacuolar Protein Sorting 10 protein (Vps10) family of receptors. Sortilin (also known as the neurotensin receptor 3; Accession number NP_002950, herein incorporated by reference) is expressed abundantly in the central and peripheral nervous systems and is also expressed in other types of tissues. For example, the expression of sortilin is upregulated in a number of cancers including for example ovarian, breast, colon and prostate cancer. The encoded preproprotein is proteolytically processed by furin to generate the mature receptor with a molecular weight of 100-110 kDa. A truncated and soluble form of Sortilin (95 kDa) has also been described, corresponding to its large luminal domain (i.e. extracellular domain or ectodomain), which has been previously detected in the supernatant medium from sortilin-overexpressing cells (48). Amino acid residues of sortilin referenced herein correspond to positions in the full-length form (i.e. Accession number NP_002950). The extracellular domain of Sortilin is at amino acid residues 78-755 of the full-length form. The peptide compounds, conjugate compounds, antibodies and conjugate antibodies herein described can have a high binding affinity to sortilin and thus can specifically target cancer cells expressing or overexpressing sortilin.

The term “compound” as used in the present document refers to compounds of formulas (I), (II), (III), (IV), (V), (VII), (VII), (VIII), (IX), (X), (XI), (XII), (XIII), (XIV), (XV), (XVI), (XVII), (XIX), (XXIII), (XXVI), (XXVIII), (LI), (LII) or to pharmaceutically acceptable salts, solvates, hydrates and/or prodrugs of these compounds, isomers of these latter compounds, or racemic mixtures of these latter compounds, and/or to composition(s) made with such compound(s) as previously indicated in the present disclosure. The expression “compound” also refers to mixtures of the various compounds herein disclosed.

Compounds of the present disclosure include prodrugs. In general, such prodrugs will be functional derivatives of these compounds which are readily convertible in vivo into the compound from which it is notionally derived. Prodrugs of the compounds of the present disclosure may be conventional esters formed with available hydroxy, or amino group. For example, an available OH or nitrogen in a compound of the present disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the present disclosure are those in which one or more of the hydroxy groups in the compounds are masked as groups which can be converted to hydroxy groups in vivo. Conventional procedures for the selection and preparation of suitable prodrugs are described, for example, in “Design of Prodrugs” ed. H. Bundgaard, Elsevier, 1985.

Compounds of the present disclosure include radiolabeled forms, for example, compounds labeled by incorporation within the structure ²H, 3H, ¹⁴C, ⁵N, or a radioactive halogen such as ¹²⁵I. A radiolabeled compound of the compounds of the present disclosure may be prepared using standard methods known in the art.

The term “analog” as used herein includes parts, extensions, substitutions, variants, modifications or chemical equivalents and derivatives thereof of the amino acid of the present disclosure that perform substantially the same function as the peptide or antigen of the disclosure in substantially the same way. For example, analogs of peptides and antigens of the disclosure include, without limitation, conservative amino acid substitutions. Analogs of the peptides and antigens of the disclosure also include additions and deletions to the peptides and antigens of the disclosure.

A “conservative amino acid substitution”, as used herein, is one in which one amino acid residue is replaced with another amino acid residue without abolishing the peptide's or antigen's desired properties.

The expression “derivative thereof” as used herein when referring to a compound means a derivative of the compound that has a similar reactivity and that could be used as an alternative to the compound in order to obtain the same desired result.

The term “cancer” as used herein means a primary or a secondary cancer and includes a non-metastatic cancer and/or a metastatic cancer. Reference to cancer includes reference to cancer tissues or cells. For example, the cancer is ovarian cancer, brain cancer, breast cancer (e.g. triple negative breast cancer), melanoma, colorectal cancer, glioblastoma, liver cancer, lung cancer, prostate cancer, cervical cancer, head cancer, gastric cancer, kidney cancer, endometrial cancer, testis cancer, urothelial cancer, acute lymphoblastic leukemia, acute myeloid leukemia, Hodgkin lymphoma, neuroblastoma, non-Hodgkin lymphoma, soft tissue cancer, bone sarcoma, thyroid cancer, transitional cell bladder cancer, Wilm's tumour, glioma, pancreatic cancer or spleen cancer. The term “cancer” as used herein also comprises any cancer involving expression of Sortilin.

The term “aggressive cancer” as used herein refers to cancer having cancer cells that are rapidly dividing and growing. Aggressive cancer may be invasive or metastatic or is more likely to be invasive or metastatic and to spread to lymph nodes and/or other body organs. Reference to aggressive cancer includes reference to aggressive cancer tissues or cells. Aggressive cancer can be any of cancer type described herein. Aggressive cancer may also exhibit characteristics such as vasculogenic mimicry.

The term “vasculogenic mimicry” (or VM) as used herein refers to the formation of microvascular channels by cancer cells. The cancer cells involved vasculogenic mimicry are typically aggressive, metastatic and genetically deregulated. Vasculogenic mimicry differs from angiogenesis in that it occurs de novo without the presence of endothelial cells, as cancer cells line tumour vessels mimicking a true vascular endothelium. There are two main types of vasculogenic mimicry: tubular and patterned. Tubular vascukogenic mimicry is morphologically similar to normal blood vessels, whereas patterned vasculogenic mimicry is visibly different although capable of undergoing anastomosis with blood vessels. Vasculogenic mimicry is found in various cancer types, for example, melanoma, ovarian cancer, breast cancer (e.g. triple negative breast cancer), prostate cancer, osteosarcoma, bladder cancer, colorectal cancer, hepatocellular cancer, gastric cancer, lung cancer, and other cancer type described herein.

The expression “therapeutic agent” as used herein means an agent capable of producing a therapeutic effect by inhibiting or decreasing angiogenic or vasculogenic mimicry in a subject, in a cancerous tissue, or in cells, compared to a control. For example, the therapeutic agent is an anti-vasculogenic mimicry agent anti-sortilin antibody described in this disclosure. The anti-sortilin antibody can be conjugated to anticancer drugs, for example, docetaxel, doxorubicin, cabazitaxel, maytansinoids, auristatins, calicheamicins, amatoxin, amanitin, and aldoxorubicin, or phytochemicals (Curcumin). The anti-vasculogenic mimicry agent can also be a peptide described herein, for example, KBP-101, KBP-102, KBP-103, KBP-104, KBP-105, KBP-106, KBP-201, KBP-202, or KBP-203. The peptide can also be conjugated to anticancer drugs, for example, docetaxel, doxorubicin, cabazitaxel, maytansinoids, auristatins, calicheamicins, amatoxins, amanitin, and aldoxorubicin, or phytochemicals (Curcumin). For example, KBC-106, KBC-201, KBP-106-Cys-Aldorubicin, Docetaxel-Katana peptide conjugate (DoceKA), or Doxorubicin-Katana peptide conjugate (DoxKA).

The term “anticancer agent” as used herein means an agent capable of causing toxicity in cancer cells. For example, taxanes, which are derived from the bark of the Pacific yew tree Taxus brevifolia, can be used as anticancer agents. Taxanes include for example docetaxel and cabazitaxel. Other anticancer agents include for example anthracycline compounds which work by intercalating DNA. For example, anthracyclines include doxorubicin and aldoxorubicin.

The term “docetaxel” or “doce” as used herein means an anticancer agent having the structure:

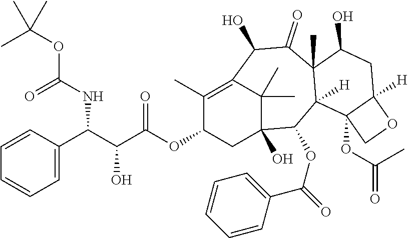

or pharmaceutically acceptable salts, solvates or prodrugs thereof as well as mixtures thereof. For example, docetaxel can be conjugated to a peptide compound or an isolated antibody of the present disclosure via the oxygen atom attached to the carbon atom at position 2 of its side chain. Docetaxel can be connected to the peptide compound or the isolated antibody directly or via a linker.

The term “doxorubicin”, “dox” or “doxo” as used herein means an anticancer agent having the structure:

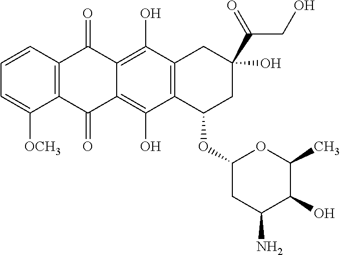 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

or pharmaceutically acceptable salts, solvates or prodrugs thereof as well as mixtures thereof. For example, doxorubicin can be conjugated to a peptide compound or an isolated antibody of the present disclosure via the oxygen atom attached to the carbon atom at position 14. Doxorubicin can be connected to the peptide compound or the isolated antibody directly or via a linker.

The term “cabazitaxel” or “cab” as used herein means an anticancer agent having the structure:

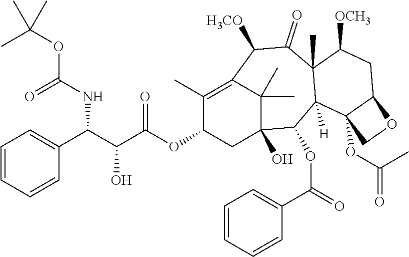 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

or pharmaceutically acceptable salts, solvates or prodrugs thereof as well as mixtures thereof. For example, cabazitaxel can be conjugated to a peptide compound or an isolated antibody of the present disclosure via the oxygen atom attached to the carbon atom at position 2 of its side chain. Cabazitaxel can be connected to the peptide compound or the isolated antibody directly or via a linker.

The term “aldoxorubicin” or “aldo” as used herein means an anticancer agent having the structure:

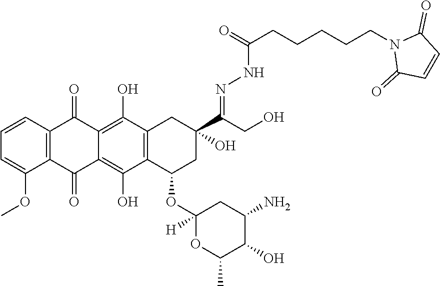 [SMILES: COc1cccc2c1C(=O)c1c(O)c3c(c(O)c1C2=O)C[C@@](O)(/C(CO)=N/NC(=O)CCCCCN1C(=O)C=CC1=O)C[C@@H]3O[C@H]1C[C@H](N)[C@H](O)[C@H](C)O1]

or pharmaceutically acceptable salts, solvates or prodrugs thereof as well as mixtures thereof. For example, aldoxorubicin can be conjugated to a peptide compound or an isolated antibody of the present disclosure via the (6-maleimidocaproyl) hydrazone attached to the carbon in position 13 of its side chain. Aldoxorubicin can be connected to the peptide compound or the isolated antibody directly or via its linker.

The term “phytochemical” as used herein means chemical compounds that occur naturally in plants and that can be used for inhibiting vasculogenic mimicry. Examples of phytochemicals include for example Curcumin. Curcumin (diferuloylmethane) is a yellow pigment present in the spice turmeric (Curcuma longa) that has been associated with anti-inflammatory. Other phytochemicals with anti-inflammatory properties include for example omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

The term “curcumin” or “cur” as used herein means a phytochemical having the structure:

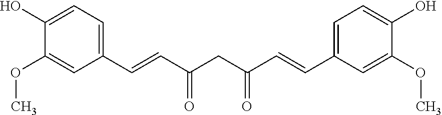 [SMILES: COc1cc(/C=C/C(=O)CC(=O)/C=C/c2ccc(O)c(OC)c2)ccc1O]

or pharmaceutically acceptable salts, solvates or prodrugs thereof as well as mixtures thereof. For example, curcumin can be conjugated to a peptide compound or an isolated antibody of the present disclosure via an oxygen atom of its phenol groups. Curcumin can be connected to the peptide compound or the isolated antibody directly or via a linker.

The expression “conjugate compounds”, “peptide-drug conjugates”, or “peptide conjugates” as used herein refers to compounds comprising a peptide compound herein disclosed connected to at least one therapeutic agent, optionally via a linker. Conjugate compound can comprise, for example, 1, 2, 3 or 4 molecules of a therapeutic agent connected thereto. These 1-4 molecules of therapeutic agent can be the same or different i.e. up to four different therapeutic agents could be connected to the peptide compounds. The therapeutic agent(s) are connected to the peptide compound via at least one covalent bond, at least one atom or at least one linker. Conjugate compounds can be used in inhibiting vasculogenic mimicry. Examples of conjugate compounds include, without limitation, the conjugate compounds shown below:

| | |
|---|---|
| KBC-106 | Acetyl-GVRAK(curcumin)AGVRN(Nle)FK(curcumin)SESY - Formula (XVI) |
| (2:1) | that comprises the peptide compound having SEQ ID NO: 15 wherein each lysine |
| | residue has a curcumin molecule connected thereto |
| KBC-201 | Acetyl-YK(curcumin)SLRRK(curcumin)APRWDAPLRDPALRQLL - Formula (XVII) |
| (2:1) | that comprises the peptide compound having SEQ ID NO: 16 wherein each lysine |
| | residue has a curcumin molecule connected thereto |

The term “antibody” as used herein refers to monoclonal antibodies including chimeric and humanized monoclonal antibodies, polyclonal antibodies, humanized antibodies, human antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. The antibodies are optionally in any useful isotype, including IgM which in one embodiment is used for diagnostic applications and IgG, such as IgG1, IgG2, IgG3 and IgG4 which in one embodiment is used for therapeutic applications.

The term “isolated antibody” refers to antibody produced in vitro or in vivo that has been removed from the source that produced the antibody, for example, an animal, hybridoma or other cell line, including recombinant cells that produce antibody. The isolated antibody is optionally “purified”, which means at least: 80%, 85%, 90%, 95%, 98% or 99% purity and optionally pharmaceutical grade purity.

The term “Anti-Sortilin mAb #1” or “Anti-SORT1 mAb #1” refers the monoclonal antibody obtained from EMB Millipore (clone F11; Cat #MABN1792). The immunogen of this antibody is in the extracellular domain of Sortilin, i.e. amino acid residues 78-755 of Sortilin.

The term “Anti-Sortilin mAb #2” or “Anti-SORT2 mAb #2” refers to the monoclonal antibody obtained from BD Bioscience (clone 48; Cat #612100). The immunogen of this antibody is amino acid residues 300-422 of Sortilin corresponding to part of the extracellular domain of this protein.

Specific antibodies, or antibody fragments, reactive against particular antigens or molecules, accessible in Sortilin, including amino acid residues 300-422 of Sortilin (SEQ ID NO: 25), may also be generated by screening expression libraries encoding immunoglobulin genes, or portions thereof, expressed in bacteria with cell surface components. For example, complete Fab fragments, VH regions and FV regions can be expressed in bacteria using phage expression libraries (see for example ref. 33). Example of antigenic Sortilin residues includes, without limitation, amino acid sequence shown below:

| | |
|---|---|
| SEQ ID NO: 25 (amino acid | GVKIYSFGLGGRFLFASVMADKDTTRRIHVSTDQGDTWSMAQL |
| residues 300-422 of sortilin) | PSVGQEQFYSILAANDDMVFMHVDEPGDTGFGTIFTSDDRGIVY |
| | SKSLDRHLYTTTGGETDFTNVTSLRGVYITSVLSED |
| SEQ ID NO: 26 (amino acid | GVKIYSFGLGGRFLFASVMADKDTTRRIHVS |
| residues 300-330 of sortilin) | |
| SEQ ID NO: 27 (amino acid | ASVMADKDTTRRIHVSTDQGDTWSMAQLPSV |
| residues 315-345 of sortilin) | |
| SEQ ID NO: 28 (amino acid | STDQGDTWSMAQLPSVGQEQFYSILAANDDM |
| residues 330-360 of sortilin) | |
| SEQ ID NO: 29 (amino acid | VGQEQFYSILAANDDMVFMHVDEPGDTGFGT |
| residues 345-375 of sortilin) | |
| SEQ ID NO: 30 (amino acid | MVFMHVDEPGDTGFGTIFTSDDRGIVYSKSL |
| residues 360-390 of sortilin) | |
| SEQ ID NO: 31 (amino acid | TIFTSDDRGIVYSKSLDRHLYTTTGGETDFT |
| residues 375-405 of sortilin) | |
| SEQ ID NO: 32 (amino acid | LDRHLYTTTGGETDFTNVTSLRGVYITSVLSED |
| residues 390-422 of sortilin) | |
| SEQ ID NO: 33 (amino acid | GVKIYSFGLGGRFLFASVMAD |
| residues 300-320 of sortilin) | |
| SEQ ID NO: 34 (amino acid | GRFLFASVMADKDTTRRIHVS |
| residues 310-330 of sortilin) | |
| SEQ ID NO: 35 (amino acid | DKDTTRRIHVSTDQGDTWSMA |
| residues 320-340 of sortilin) | |
| SEQ ID NO: 36 (amino acid | STDQGDTWSMAQLPSVGQEQF |
| residues 330-350 of sortilin) | |
| SEQ ID NO: 37 (amino acid | AQLPSVGQEQFYSILAANDDM |
| residues 340-360 of sortilin) | |
| SEQ ID NO: 38 (amino acid | FYSILAANDDMVFMHVDEPGD |
| residues 350-370 of sortilin) | |
| SEQ ID NO: 39 (amino acid | MVFMHVDEPGDTGFGTIFTSD |
| residues 360-380 of sortilin) | |
| SEQ ID NO: 40 (amino acid | DTGFGTIFTSDDRGIVYSKSL |
| residues 370-390 of sortilin) | |
| SEQ ID NO: 41 (amino acid | DDRGIVYSKSLDRHLYTTTGG |
| residues 380-400 of sortilin) | |
| SEQ ID NO: 42 (amino acid | LDRHLYTTTGGETDFTNVTSL |
| residues 390-410 of sortilin) | |
| SEQ ID NO: 43 (amino acid | GETDFTNVTSLRGVYITSVLSED |
| residues 400-422 of sortilin) | |
| SEQ ID NO: 44 (amino acid | ADKDTT |
| residues 319-324 of sortilin) | |
| SEQ ID NO: 45 (amino acid | STDQGDTWS |
| residues 330-338 of sortilin) | |
| SEQ ID NO: 46 (amino acid | LPSVGQE |
| residues 342-348 of sortilin) | |
| SEQ ID NO: 47 (amino acid | DEPGDTGF |
| residues 366-373 of sortilin) | |
| SEQ ID NO: 48 (amino acid | TS |
| residues 378-379 of sortilin) | |
| SEQ ID NO: 49 (amino acid | RGI |
| residues 382-384 of sortilin) | |
| SEQ ID NO: 50 (amino acid | TTTGGETDFT |
| residues 396-405 of sortilin) | |

In some embodiments, the isolated antibody that specifically binds to a polypeptide having or comprising the amino acid sequence as shown in any one of SEQ ID NOs: 25-50, an analog thereof, or a fragment thereof, is for use in inhibiting vasculogenic mimicry. In some embodiments, the isolated antibody targets Sortilin receptor. In other embodiments, the isolated antibody binds at least 2, optionally at least 4 contiguous amino acid residues as shown in any one of SEQ ID NOs: 25-50, an analog thereof, or a fragment thereof.

The term “humanized antibody” as used herein means that the antibody or fragment comprises human conserved framework regions (alternatively referred to as constant regions) and the hypervariable regions (alternatively referred to as the antigen binding domain) are of non-human origin. For example, the hypervariable region may be from a mouse, rat or other species. The humanization of antibodies from non-human species has been well described in the literature. See for example Carter & Merchant 1997 (34). Humanized antibodies are also readily obtained commercially.

Humanized forms of rodent antibodies are readily generated by CDR grafting (35). In this approach the six CDR loops comprising the antigen binding site of the rodent monoclonal antibody are linked to corresponding human framework regions. CDR grafting often yields antibodies with reduced affinity as the amino acids of the framework regions may influence antigen recognition (36). To maintain the affinity of the antibody, it is often necessary to replace certain framework residues by site directed mutagenesis or other recombinant techniques and may be aided by computer modeling of the antigen binding site (37). Humanized forms of antibodies are optionally obtained by resurfacing (38). In this approach only the surface residues of a rodent antibody are humanized.

Humanized antibodies are selected from any class of immunoglobulins including: IgM, IgG, IgD, IgA or IgE; and any isotype, including: IgG1, IgG2, IgG3 and IgG4. The humanized or human antibody may include sequences from one or more than one isotype or class. Further, these antibodies are typically produced as antigen binding fragments such as Fab, Fab′ F(ab′)2, Fd, Fv and single domain antibody fragments, or as single chain antibodies in which the heavy and light chains are linked by a spacer. Also, the humanized antibodies may exist in monomeric or polymeric form. The humanized antibody optionally comprises one non-human chain and one humanized chain (i.e. one humanized heavy or light chain).

Additionally, antibodies specific for polypeptides having or comprising the amino acid sequence as shown in any one of SEQ ID NOs: 25-50, e.g. amino acid residues 300-422 of sortilin (Accession number NP_002950), are readily isolated by screening antibody phage display libraries. For example, an antibody phage library is optionally screened by using parts of the antigen of the current disclosure to identify antibody fragments specific for sortilin. Antibody fragments identified are optionally used to produce a variety of recombinant antibodies that are useful with different embodiments of the present disclosure. Antibody phage display libraries are commercially available, for example, through Xoma (Berkeley, Calif.) Methods for screening antibody phage libraries are well known in the art.

Other methods for generating antibodies are known in the art. For example, an antigenic polypeptide having or comprising the amino acid sequence as shown in any one of SEQ ID NOs: 25-50, an analog thereof, or a fragment thereof, can be conjugated to KLH for immunization of, for example, BALB/c mice to generate B cells reactive to epitopes on the polypeptide. Alternatively a portion of the polypeptide having or comprising the amino acid sequence of any of the SEQ ID NOs: 25-50 peptides comprising one or more antigenic determinants is conjugated to KLH, minimally comprising 3 or 5 contiguous amino acids of any of the peptide sequence that is immunogenic either alone or when coupled to KLH.

After immunization of an animal with an antigenic preparation of a Sortilin polypeptide (e.g. amino acid sequence as shown in SEQ ID NO: 25-50, an analog thereof, or a fragment thereof), antisera can be obtained and polyclonal antibodies can be isolated from the serum. For production of monoclonal antibodies, antibody-producing cells (i.e. B-lymphocytes) can be harvested from an immunized animal and fused by somatic cell fusion procedures well known in the art with immortalizing cells such as myeloma cells to yield hybridoma cells. Such techniques include, for example, the hybridoma technique (39), the human B cell hybridoma technique (40), and the EBV-hybrdoma technique to produce human monoclonal antibodies (41). Hybridoma cells can be screened immunochemically for production of antibodies specifically bind with a Sortilin polypeptide and monoclonal antibodies isolated from a culture comprising such hybridoma cells.

The term “specifically binds” or derivative thereof as used herein in reference to an antibody is intended to mean, as is generally understood in the art, that the antibody is sufficiently selective between the antigen of interest (e.g., a Sortilin polypeptide) and other antigens that are not of interest that the antibody is useful for detecting the presence of the antigen of interest in a particular type of biological sample. Without wishing to be bound by theory, in certain methods employing the antibody, for example, therapeutic applications, a higher degree of specificity in binding may be desirable. Monoclonal antibodies generally have a greater tendency than polyclonal antibodies in discriminating effectively between the desired antigens and cross-reacting polypeptides. Affinity of the antibody for the antigen, which is usually expressed by a dissociation constant, is a characteristic that influences the specificity of an antibody:antigen interaction. The desired specificity may be reached with a range of different affinities, and in general preferred antibodies will have a dissociation constant of about 10-, 10-7, 10-, 10-9 or less. In another example, the antibody can bind 3-5, 5-7, 7-10, 10-15, 5-15, or 5-30 fold more efficiently to its antigen of interest compared to another molecule.

Further, the properties of the antibody obtained are influenced by the techniques used to screen antibodies. For example, if an antibody is to be used for binding an antigen in solution, then solution binding may be carried out. Different techniques are available for testing interaction between antibodies and antigens to identify particularly desirable antibodies. Such techniques include western blots, immunoprecipitation assays, immunohistochemistry, ELISAs, surface plasmon resonance binding assays such as the Biacore binding assay (Bia-core AB, Uppsala, Sweden), and sandwich assays such as paramagnetic bead system.

In an aspect, the disclosure provides antibodies that bind to a soluble Sortilin polypeptide. Such antibodies may be generated as described herein, using a soluble Sortilin polypeptide sequence as shown in any one of SEQ ID NOs: 25-50, an analog or a fragment thereof as the antigen. Antibodies of this type can be used, e.g., to detect Sortilin polypeptides in biological samples and/or to monitor soluble Sortilin polypeptide levels in a subject. In another aspect, an antibody that specifically binds to a soluble Sortilin polypeptide can be used to modulate activity of Sortilin polypeptide and associated pathway, thereby inhibiting vasculogenic mimicry.

The expression “antibody-drug conjugates”, antibody conjugates or “conjugate antibody” as used herein refers to an antibody herein disclosed connected to at least one therapeutic agent, optionally via a linker. Antibody-drug conjugates can comprise, for example, 1, 2, 3, 4, 5, 6, 7, 8, 9, 10, 11, or 12 molecules of a therapeutic agent connected thereto. These 1-12 molecules of therapeutic agent can be the same or different i.e. up to four different therapeutic agents could be connected to the antibody. The therapeutic agent(s) are connected to the antibody via at least one covalent bond, at least one atom or at least one linker. Antibody-drug conjugate can be used in inhibiting vascusogenic mimicry. Examples of antibody-drug conjugate include, without limitation, anti-Sortilin antibody conjugated with anticancer drugs such as docetaxel, doxorubicin, cabazitaxel, maytansinoids, aurstatine, calicheamicins, amatoxins, amanitin, and aidoxorubicin, and/or phytochemicals such as Curcumin. The following table summarizes anticancer drugs that can be conjugated with an anti-Sortilin antibody:

A) Cytotoxics (Cytotoxic agents)

| | |
|---|---|
| 1. | Alkylating agent |
| 2. | Platinum coordination: Cisplatin, Carboplatin, Oxaliplatin |
| 3. | Antimetabolites |
| 4. | Microtubule damaging agents: Vincristine, Vinblastine, Vinorelbine, Paclitaxel, Docetaxel |
| 5. | Topoisomerase-2 inhibitor: Etoposide |
| 6. | Topoisomerase-1 inhibitor: Topotecan, Irinotecan |
| 7. | Antibiotics: Actinomycin D, Doxorubicin, Daunorubicin, Epirubicin, Bleomcins, Mitomycin C |
| 8. | Miscelleneous: Hydroxyurea, L-Asparaginase, Tretinoin |

B) Toxins

| | |
|---|---|
| 1. | Maytansinoids |
| 2. | Auristatins |
| 3. | Calicheamicins |
| 4. | Amatoxin |
| 5. | Amanitin |

C) Anticancer peptides

| | |
|---|---|
| 1. | D-peptide A, B, C and D, D-K6L9, NRC-03, NRC-07, Gomesin, Hepcidin TH2-3, Dermaseptin |
| | B2, PTP7, MGA2, HNP-1, Tachyplesin, Temporin-1 CEa, NK-2, Cecropin CB1 |

D) Targeted drugs

| | |
|---|---|
| 1. | Tyrosine protein kinase inhibitors: Imatinib, Dasatinib |
| 2. | EFG receptor inhibitor: Gefitnib, Erlotinib |
| 3. | Angiogenesis inhibitor: Bevacizumab, Thalidomide, Endostatin, Angiostatin, Angiopoitein, |
| | Cannabionoids |
| 4. | Proteasome inhibitor: Bortezomib, Carfizomib, Izazomib, Marizomib, Epoxomicin |
| 5. | Monoclonal antibodies (mAbs): Rituximab, Trastuzumab |

E) Immunotherapies

| | |
|---|---|
| 1. | Check point inhibitors |
| 2. | CAR-T cell therapy |
| 3. | Antibodies |
| 4. | Antibody drug conjugate |
| 5. | Bispecific T cell engagers and Bispecific antibodies |
| 6. | Genetically Engineered T cell-mediated cell killing |
| 7. | Oncolytic viruses |
| 8. | T-cell mediated cytolysis |

F) Phytochemicals

| | |
|---|---|
| 1. | Alkaloids: Chlorogenic acid, Theobromine,Theophylline |
| 2. | Anthocyanins: Cyanidin, Malvidin |
| 3. | Carotenoids: Beta-Carotene, Lutein, Lycopene |
| 4. | Coumestans |
| 5. | Flavan-3-Ols |
| 6. | Flavonoids: Epicatechin, Hesperidin, Isorhamnetin, Kaempferol, Myricetin, Naringin, |
| | Nobiletin, Proanthocyanidins, Quercetin, Rutin, Tangeretin |
| 7. | Hydroxycinnamic Acids: Chicoric acid, Coumarin, Ferulic acid, Scopoletin |
| 8. | Isoflavones: Daidzein, Genistein |
| 9. | Lignans: Silymarin |
| 10. | Monoterpenes: Geraniol, Limonene |
| 11. | Organosulfides: Allicin, Glutathione, lndole-3-Carbinol, Isothiocyanates, Sulforaphane |
| 12. | Other Phytochemicals: Damnacanthal, Digoxin, Phytic acid |
| 13. | Phenolic Acids: Capsaicin, Ellagic Acid, Gallic acid, Rosmarinic acid, Tannic Acid |
| 14. | Phytosterols: Beta-Sitosterol |
| 15. | Saponins |
| 16. | Stylbenes: Pterostilbene, Resveratrol |
| 17. | Triterpenoids: Ursolic acid |
| 18. | Xanthophylls: Astaxanthin, Beta-Cryptoxanthin |
| 19. | Monophenols: Hydroxytyrosol |

G) oligopeptidomimetics

| | |
|---|---|
| 1. | Tubulysins |

The term “conjugating” as used herein, refers, for example, to the preparation of a conjugate as defined above. Such an action comprises connecting a peptide compound or an antibody together with at least one therapeutic agent, optionally via a linker.

The term “CD133 positive cell” or “CD133 positive cells” as used herein refers to a cell or cells expressing the CD133 cell surface marker.

For example, the following are general chemical formulas of some peptide-conjugate compounds herein disclosed.

Curcumin-Katana conjugate compound:

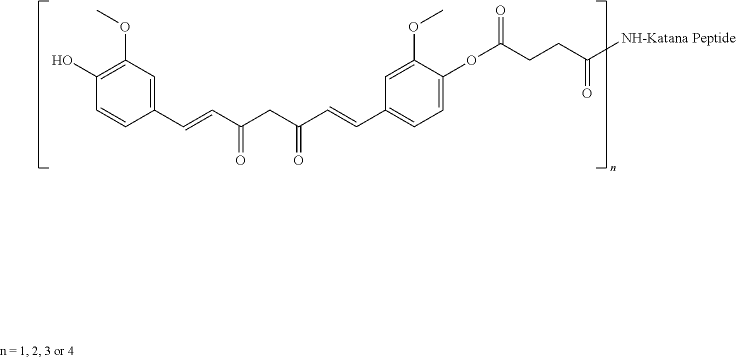

For example, the following are the chemical structures of some conjugate compounds herein disclosed.

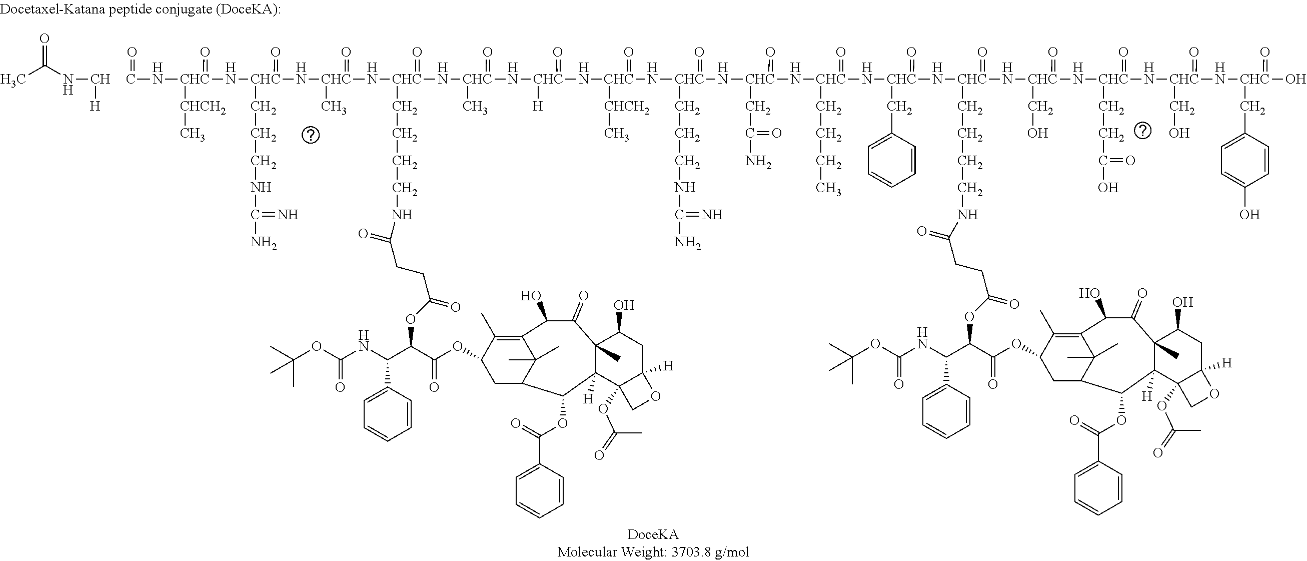

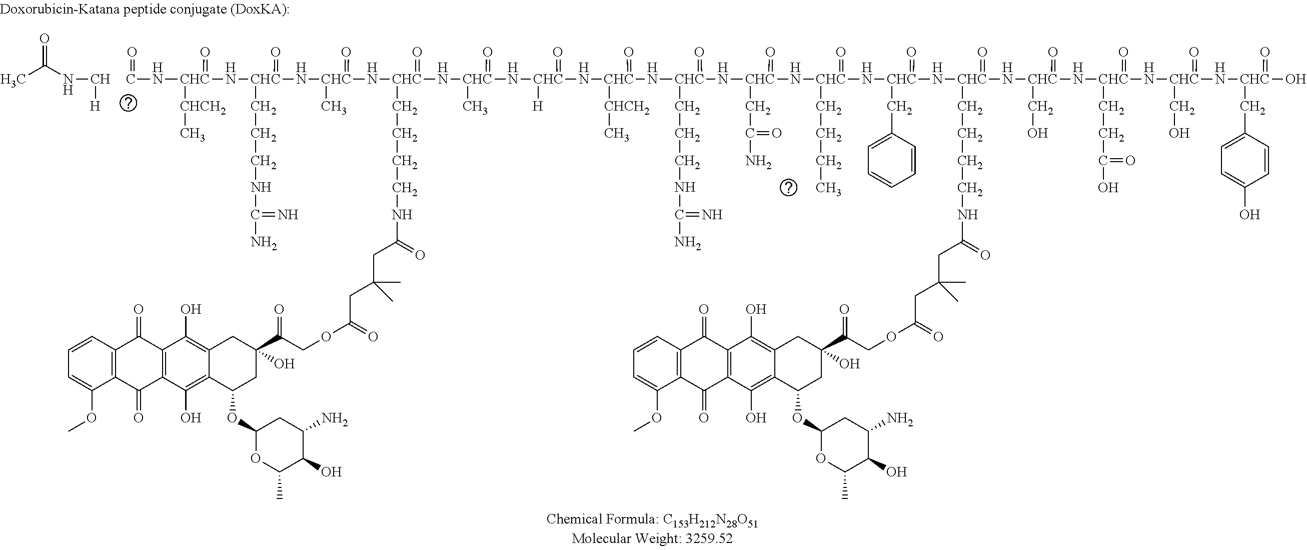

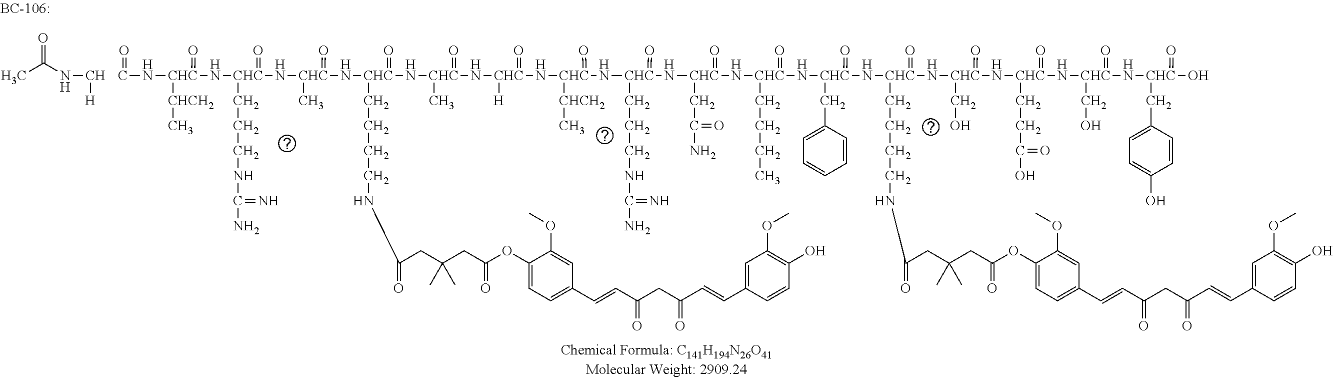

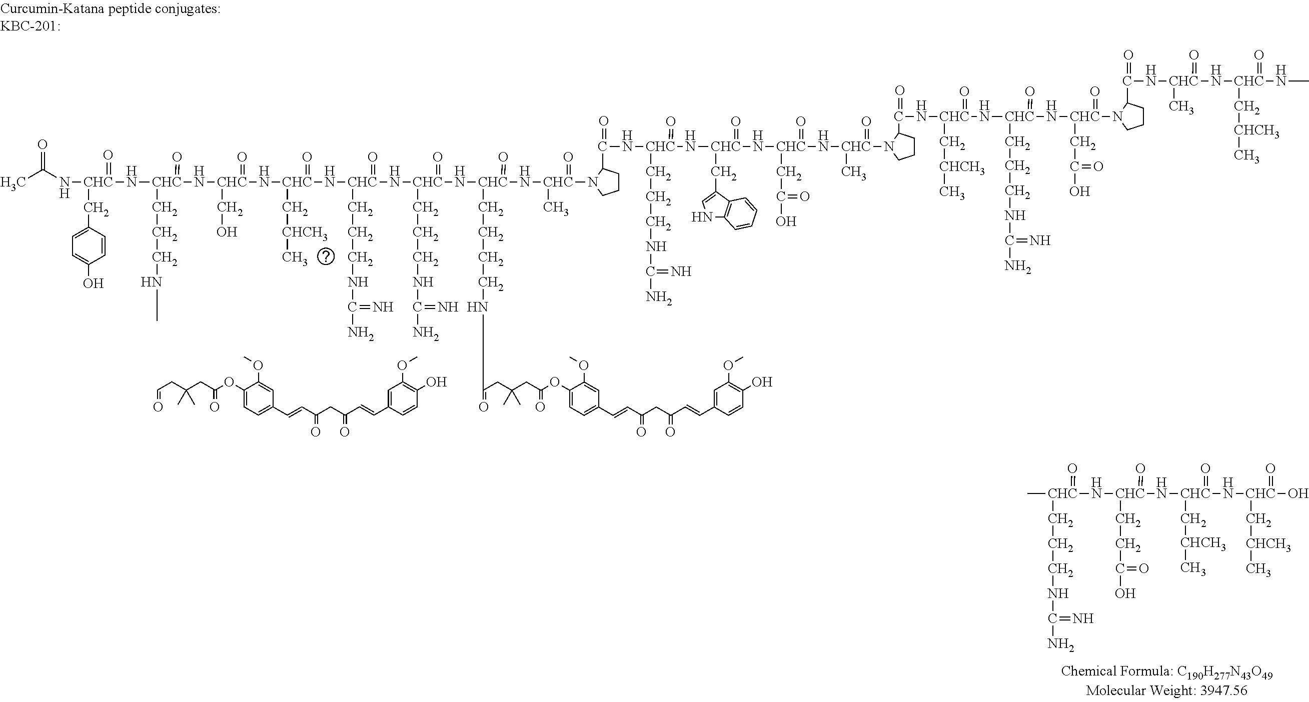

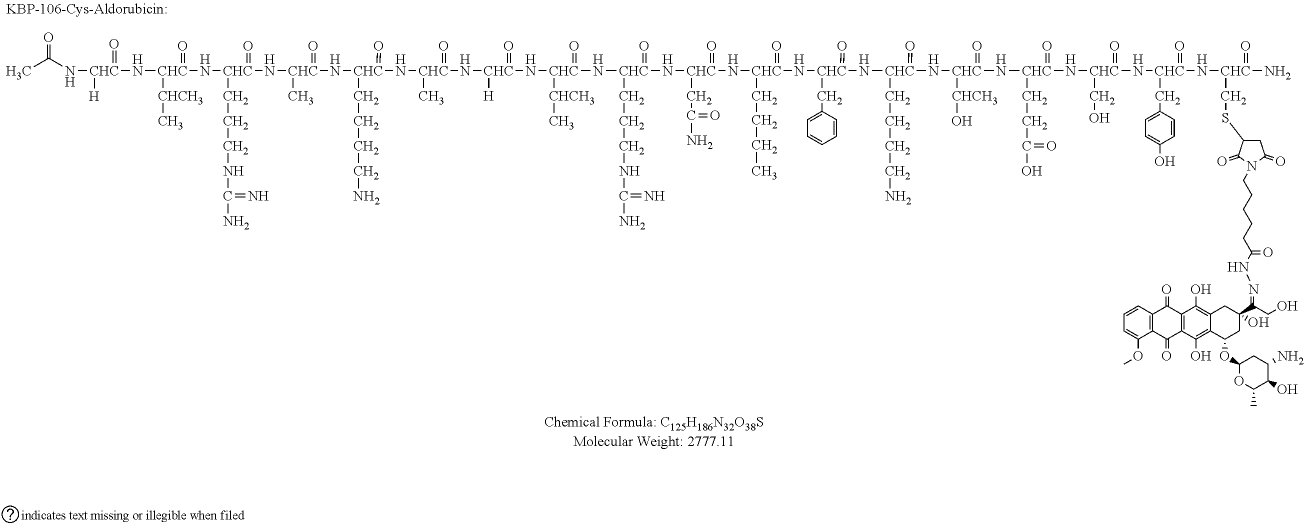

The term “linker” as used herein means a chemical structure connecting a peptide compound or an antibody herein disclosed to at least one therapeutic agent. The linker can be connected to the peptide compound or the isolated antibody at different functional groups on the peptide compound or antibody. For example, the linker can be connected to the peptide compound or the isolated antibody at the primary amines (amines (—NH2): this group exists at the N-terminus of each polypeptide chain (called the alpha-amine) and in the side chain of lysine (Lys, K) residues (called the epsilon-amine). For example, the linker can be connected to the peptide compound or the isolated antibody at the carboxyls (—COOH): this group exists at the C-terminus of each polypeptide chain and in the side chains of aspartic acid (Asp, D) and glutamic acid (Glu, E). For example, the linker can be connected to the peptide compound or the isolated antibody at the Sulfhydryls (—SH): This group exists in the side chain of cysteine (Cys, C). Often, as part of a protein's secondary or tertiary structure, cysteines are joined together between their side chains via disulfide bonds (—S—S—). These must be reduced to sulfhydryls to make them available for crosslinking by most types of reactive groups. For example, the linker can be connected to the peptide compound or the isolated antibody at the Carbonyls (—CHO): Ketone or aldehyde groups can be created in glycoproteins by oxidizing the polysaccharide post-translational modifications (glycosylation) with sodium meta-periodate. For example, the linker can be a cleavable linker. For example, the linker can be a non-cleavable linker.

The following table summarizes the reactivity class and the chemical group of some of the principal linkers for standard chemical conjugation:

| Reactivity class | Chemical group |
|---|---|
| Carboxyl-to-amine reactive groups | Carbodiimide (e.g., EDC) |
| Amine-reactive groups | NHS ester |
| | Imidoester |
| | Pentafluorophenyl ester |
| | Hydroxymethyl phosphine |
| Sulfhydryl-reactive groups | Maleimide |
| | Haloacetyl (Bromo- or Iodo-) |
| | Pyridyldisulfide |
| | Thiosulfonate |
| | Vinylsulfone |
| Aldehyde-reactive groups | Hydrazide |
| i.e., oxidized sugars (carbonyls) | Alkoxyamine |
| Photoreactive groups | Diazirine |
| | Aryl Azide |

For example, homobifunctional and heterobifunctional crosslinkers can be used. For example, Disuccinimidyl suberate (DSS) is a homobifunctional crosslinker that has identical amine-reactive NHS-ester groups at either end of a short spacer arm. For example, Sulfosuccinimidyl 4-(N-maleimidomethyl)cyclohexane-1-carboxylate (Sulfo-SMCC) is a heterobifunctional crosslinker that has an amine-reactive sulfo-NHS-ester group at one end and a sulfhydryl reactive maleimide group at the opposite end of a cyclohexane spacer arm. This allows for sequential, two-step conjugation procedures. Among the commercially available homobifunctional cross-linkers are: BSOCOES (Bis(2-[Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); and BASED (Bis(s-[4-azidosalicylamido]-ethyl)disulfide iodinatable).

The peptide compounds or antibodies may be conjugated through a variety of linkers, e.g., sulfhydryl groups, amino groups (amines), or any appropriate reactive group. The linker can be a covalent bond. The linker group may comprise a flexible arm, e.g., 2, 3, 4, 5, 6, 7, 8, 9, 10, 11, 12, 13, 14, or 15 carbon atoms.

Exemplary linkers include, without limitation, pyridinedisulfide, thiosulfonate, vinylsulfonate, isocyanate, imidoester, diazine, hydrazine, thiol, carboxylic acid, multi-peptide linkers, and acetylene. Alternatively other linkers that can be used include BS³ [Bis(sulfosuccinimidyl)suberate] (which is a homobifunctional N-hydroxysuccinimide ester that targets accessible primary amines), NHS/EDC (N-hydroxysuccinimide and 1-ethyl-3-(3-dimethylaminopropyl)carbodiimide (NHS/EDC allows for the conjugation of primary amine groups with carboxyl groups), sulfo-EMCS ([N-ε-maleimidocaproic acid]hydrazide (sulfo-EMCS are heterobifunctional reactive groups that are reactive toward sulfhydryl and amino groups), hydrazide (most proteins contain exposed carbohydrates and hydrazide is a useful reagent for linking carboxyl groups to primary amines).

To form covalent bonds, one can use as a chemically reactive group a wide variety of active carboxyl groups (e.g., esters) where the hydroxyl moiety is physiologically acceptable at the levels required to modify the peptide compound or the antibody. Particular agents include for example N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA), maleimido hexanoic acid (MHA), and maleimido undecanoic acid (MUA).

Primary amines are the principal targets for NHS esters; NHS esters react with primary amines to form covalent amide bonds. Accessible α-amine groups present on the N-termini of proteins and the ε-amine of lysine react with NHS esters. Thus, conjugated compounds herein disclosed can include a linker having a NHS ester conjugated to an N-terminal amino of a peptide compound or an antibody, or to an ε-amine of lysine. An amide bond is formed when the NHS ester reacts with primary amines releasing N-hydroxysuccinimide. Succinimide containing reactive groups may be referred to more simply as succinimidyl groups. In some embodiments, the functional group on the peptide compound or antibody will be a thiol group and the chemically reactive group will be a maleimido-containing group such as gamma-maleimide-butylamide (GMBA or MPA). Such maleimide-containing groups may be referred to herein as maleido groups.

Amine-to-amine linkers include NHS esters, imidoesters, and others, examples of which are listed below.

Exemplary NHS esters:
DSG (disuccinimidyl glutarate)
DSS (disuccinimidyl suberate)
BS³ (bisfsulfosuccinimidyl] suberate)
TSAT (tris-succinimidyl aminotriacetate)
Variants of bis-succinimide ester-activated compounds including a polyethylene glycol
spacer such as BS(PEG)ₙ where n is 1-20 (e.g., BS(PEG)₅ and BS(PEG)₉)
DSP (Dithiobis[succinimidyl propionate])
DTSSP (3,3′-dithiobis[sulfosuccinimidylpropionate])
DST (disuccinimidyl tartarate)
BSOCOES (bis[2-(succinimidooxycarbonyloxy)ethyl]sulfone)
EGS (ethylene glycol bisfsuccinimidylsuccinate])
sulfo-EGS (ethylene glycol bis[sulfosuccinimidylsuccinate])
Exemplary imidoesters:
DMA (dimethyl adipimidate•2 HCl)
DMP (dimethyl pimelimidate•2 HCl)
DMS (dimethyl suberimidate•2 HCl)
DTBP (dimethyl 3,3′-dithiobispropionimidate•2 HCl)
Other exemplary amine-to-amine linkers:
DFDNB (1,5-difluoro-2,4-dinitrobenzene)
THPP (β-[tris(hydroxymethyl) phosphinol propionic acid (betaine))

The linker may also be a sulfhydryl-to-sulfhydryl linker, such as the maleimides and pyrdyldithiols listed below.

| Exemplary maleimides: | Another sulfhydryl linker: |
|---|---|
| BMOE (bis-maleimidoethane) | HBVS (1,6-hexane-bis-vinylsulfone) |
| BMB (1,4-bismaleimidobutane) | |
| BMH (bismaleimidohexane) | |
| TMEA (tris[2-maleimidoethyl]amine) | |
| BM(PEG)2 1,8-bis-maleimidodiethyleneglycol) | |
| BM(PEG)ₙ, where n is 1 to 20 (e.g., 2 or 3) | |
| BMDB (1,4 bismaleimidyl-2,3-dihydroxybutane) | |
| DTME (dithio-bismaleimidoethane) | |

Exemplary pyridyldithiol:

DPDPB (1,4-di-[3′-(2′-pyridyldithio)-propionamido]butane)

The linker may be an amine-to-sulfhydryl linker, which includes NHS ester/maleimide compounds. Examples of these compounds are provided below.

Amine-to-sulfhydryl linkers:
AMAS (N-(α-maleimidoacetoxy)succinimide ester)
BMPS (N-[β-maleimidopropyloxy]succinimide ester)
GMBS (N-[γ-maleimidobutyryloxy]succinimide ester)
sulfo-GMBS (N-[γ-maleimidobutyryloxy]sulfosuccinimide ester)
MBS (m-maleimidobenzoyl-N-hydroxysuccinimide ester)
sulfo-MBS (m-maleimidobenzoyl-N-hydroxysulfosuccinimide ester)
SMCC (succinimidyl 4-[N-maleimidomethyl]cyclohexane-1-carboxylate)
sulfo-SMCC (Sulfosuccinimidyl 4-[N-maleimidomethyl]cyclohexane-1-carboxylate)
EMOS ([N-ε-maleimidocaproyloxy]succinimide ester)
Sulfo-EMCS ([N-ε-maleimidocaproyloxy]sulfosuccinimide ester)
SMPB (succinimidyl 4-[p-maleimidophenyl]butyrate)
sulfo-SMPB (sulfosuccinimidyl 4-[p-maleimidophenyl]butyrate)
SMPH (succinimidyl-6-[3-maleimidopropionamido]hexanoate)
LC-SMCC (succinimidyl-4-[N-maleimidomethyl]cyclohexane-1-carboxy-[6-amidocaproate])
sulfo-KMUS (N-[κ-maleimidoundecanoyloxy]sulfosuccinimide ester)
SM(PEG)ₙ (succinimidyl-([N-maleimidopropionamido-polyethyleneglycol) ester),
where n is 1 to 30 (e.g., 2, 4, 6, 8, 12, or 24)
SPDP (N-succinimidyl 3-(2-pyridyldithio)-propionate)
LC-SPDP (succinimidyl 6-(3-[2-pyridyldithio]-propionamido)hexanoate)
sulfo-LC-SPDP (sulfosuccinimidyl 6-(3′-[2-pyridyldithio]-propionamido)hexanoate)
SMPT (4-succinimidyloxycarbonyl-α-methyl-α-[2-pyridyldithio]toluene)
Sulfo-LC-SMPT (4-sulfosuccinimidyl-6-[α-methyl-α-(2-pyridyldithio)toluamido]hexanoate)
SIA (N-succinimidyl iodoacetate)
SBAP (succinimidyl 3-[bromoacetamido]propionate)
SIAB (N-succinimidyl[4-iodoacetyl]aminobenzoate)
sulfo-SIAB (N-sulfosuccinimidyl[4-iodoacetyl]aminobenzoate)

The linker can react with an amino group and a non-selective entity. Such linkers include NHS ester/aryl azide and NHS ester/diaziine linkers, examples of which are listed below.

NHS ester/aryl azide linkers:
NHS-ASA (N-hydroxysuccinimidyl-4-azidosalicylic acid)
ANB-NOS (N-5-azido-2-nitrobenzoyloxysuccinimide)
sulfo-HSAB (N-hydroxysulfosuccinimidyl-4-azidobenzoate)
sulfo-NHS-LC-ASA (sulfosuccinimidyl[4-azidosalicylamido]hexanoate)
SANPAH (N-succinimidyl-6-(4′-azido-2′-nitrophenylamino)hexanoate)
sulfo-SANPAH (N-sulfosuccinimidyl-6-(4′-azido-2′-nitrophenylamino)hexanoate)
sulfo-SFAD (sulfosuccinimidyl-(perfluoroazidobenzamido)-ethyl-1,3′-dithioproprionate)
sulfo-SAND (sulfosuccinimidyl-2-(m-azido-o-nitrobenzamido)-ethyl-1,3′-proprionate)
sulfo-SAED (sulfosuccinimidyl 2-[7-amino-4-methylcoumarin-3-acetamido]ethyl-
1,3′dithiopropionate)
NHS ester/diazirine linkers:
SDA (succinimidyl 4,4′-azipentanoate)
LC-SDA (succinimidyl 6-(4,4′-azipentanamido)hexanoate)
SDAD (succinimidyl 2-([4,4′-azipentanamido]ethyl)-1,3′-dithioproprionate)
sulfo-SDA (sulfosuccinimidyl 4,4′-azipentanoate)
sulfo-LC-SDA (sulfosuccinimidyl 6-(4,4′-azipentanamido)hexanoate)
sulfo-SDAD (sulfosuccinimidyl 2-([4,4′-azipentanamido]ethyl)-1,3′-dithioproprionate)

Exemplary amine-to-carboxyl linkers include carbodiimide compounds (e.g., DCC (N,N-dicyclohexylcarbodimide) and EDC (1-ethyl-3-[3-dimethylaminopropyglcarbodiimide)). Exemplary sulfhydryl-to-nonselective linkers include pyridyldithiol/aryl azide compounds (e.g., APDP ((N-[4-(p-azidosalicylamido)butyl]-3′-(2′-pyridyldithio)propionamide)). Exemplary sulfhydryl-to-carbohydrate linkers include maleimide/hydrazide compounds (e.g., BMPH (N-[β-maleimidopropionic acid]hydrazide), EMCH ([N-ε-maleimidocaproic acid]hydrazide), MPBH 4-(4-N-maleimidophenyl)butyric acid hydrazide), and KMUH (N-[κ-maleimidoundecanoic acid]hydrazide)) and pyridyldithiol/hydrazide compounds (e.g., PDPH (3-(2-pyridyldithio)propionyl hydrazide)). Exemplary carbohydrate-to-nonselective linkers include hydrazide/aryl azide compounds (e.g., ABH (p-azidobenzoyl hydrazide)). Exemplary hydroxyl-to-sulfhydryl linkers include isocyanate/maleimide compounds (e.g., (N-[p-maleimidophenyg]isocyanate)). Exemplary amine-to-DNA linkers include NHS ester/psoralen compounds (e.g., SPB (succinimidyl-[4-(psoralen-8-yloxy)]-butyrate)).

To generate a branch point of varying complexity in a conjugate peptide compound or antibody, the linker can be capable of linking 3-7 entities.

Exemplary tri-functional linkers:

| | |
|---|---|
| 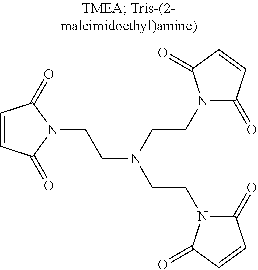 [SMILES: O=C1C=CC(=O)N1CCN(CCN1C(=O)C=CC1=O)CCN1C(=O)C=CC1=O] | LC-TSAT (tris-succinimidyl (6- aminocaproyl)aminotriacetate), tris- succinimidyl-1,3,5-benzenetricarboxylate MDSI (maleimido-3,5-disuccinimidyl isophthalate) |
| 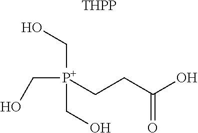 [SMILES: O=C(O)CC[P+](CO)(CO)CO ; "THPP"] | |
| 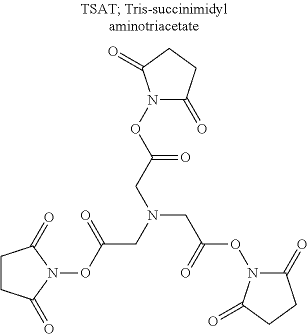 [SMILES: O=C(CN(CC(=O)ON1C(=O)CCC1=O)CC(=O)ON1C(=O)CCC1=O)ON1C(=O)CCC1=O] | SDMB (succinimidyl-3,5- dimaleimidophenyl benzoate Mal-4 (tetrakis-(3-maleimidopropyl) pentaerythritol, NHS-4 (tetrakis-(N- succinimidylcarboxypropyl) pentaerythritol)) |

TMEA and TSAT reach through their maleimide groups with sulfhydryl groups. The hydroxyl groups and carboxy group of THPP can react with primary or secondary amines. Other useful linkers conform to the formula Y═C═N-Q-A-C(O)—Z, where Q is a homoaromatic or heteroaromatic ring system; A is a single bond or an unsubstituted or substituted divalent C₁₋₃₀ bridging group, Y is O or S; and Z is Cl, Br, I, N₃, N-succinimidyloxy, imidazolyl, 1-benzotriazolyloxy, OAr where Ar is an electron-deficient activating aryl group, or OC(O)R where R is -A-Q-N═C═Y or C₄-20 tertiary-alkyl (see U.S. Pat. No. 4,680,338).

Other useful linkers have the formula

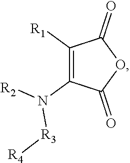

where R₁ is H, C₁₋₆ alkyl, C₂₋₆ alkenyl, C₆₋₁₂ aryl or aralkyl or these coupled with a divalent organic

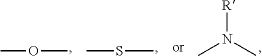

where R′ is C₁₋₆ alkyl, linking moiety; R₂ is H, C₁₋₁₂ alkyl, C₆₋₁₂ aryl, or C₆₋₁₂ aralkyl, R₃ is

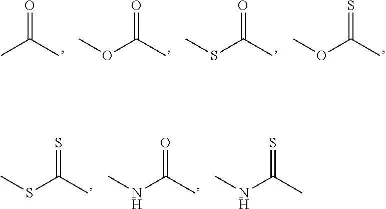

or another chemical structure that is able to delocalize the lone pair electrons of the adjacent nitrogen and R₄ is a pendant reactive group capable of linking R₃ to a peptide compound, an antibody or to an agent (see for example U.S. Pat. No. 5,306,809).

The linker may include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly or Cys). Where the linker is a short peptide, it can be a glycine-rich peptide (which tend to be flexible) such as a peptide having the sequence [Gly-Gly-Gly-Gly-Ser]n where n is an integer from 1 to 6, inclusive (see U.S. Pat. No. 7,271,149) or a serine-rich peptide linker (see U.S. Pat. No. 5,525,491). Serine rich peptide linkers include those of the formula [X-X-X-X-Gly]_{y} where up to two of the X are Thr, the remaining X are Ser, and y is an integer from 1 to 5, inclusive (e.g., Ser-Ser-Ser-Ser-Gly, where y is greater than 1). Other linkers include rigid linkers (e.g., PAPAP and (PT)ₙP, where n is 2, 3, 4, 5, 6, or 7) and α-helical linkers (e.g., A(EAAAK)ₙA, where n is 1, 2, 3, 4, or 5).

The linker can be an aliphatic linker (e.g., with an amide bond to the polypeptide and an ester bond to the therapeutic agent). Where an aliphatic linker is used, it may vary with regard to length (e.g. C₁-C₂₀) and the chemical moieties it includes (e.g., an amino group or carbamate).

Examples of suitable amino acid linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

The linker can also be a branched polypeptide. Exemplary branched peptide linkers are described in U.S. Pat. No. 6,759,509, herein incorporated by reference.

The linker can provide a cleavable linkage (e.g., a thioester linkage) or a non-cleavable linkage (e.g., a maleimide linkage). For example, a cytotoxic protein can be bound to a linker that reacts with modified free amines, which are present at lysine residues within the polypeptide and at the amino-terminus of the polypeptide. Thus, linkers useful in the present conjugate compounds can comprise a group that is reactive with a primary amine on the polypeptide or modified polypeptide to which the therapeutic agent moiety is conjugated. More specifically, the linker can be selected from the group consisting of monofluoro cyclooctyne (MFCO), bicyclo[6.1.0]nonyne (BCN), N-succinimidyl-S-acetylthioacetate (SATA), N-succinimidyl-S-acetytthiopropionate (SATP), maleimido and dibenzocyclooctyne ester (a DBCO ester). Useful cyclooctynes, within a given linker, include OCT, ALO, MOFO, DIFO, DIBO, BARAC, DIBAC, and DIMAC.

The linker may comprise a flexible arm, such as for example, a short arm (<2 carbon chain), a medium-size arm (from 2-5 carbon chain), or a long arm (3-6 carbon chain).

Click chemistry can also be used for conjugation on a peptide (DBCO, TCO, tetrazine, azide and alkyne linkers). These families of linkers can be reactive toward amine, carboxyl and sulfhydryl groups. In addition, these linkers can also be biotinylated, pegylated, modified with a fluorescent imaging dye, or phosphoramidited for incorporation onto an oligonucleotide sequence.

The term “intermediate” as used herein refers to a therapeutic agent that has been reacted with a linker thereby forming an intermediate or an activated form of the therapeutic agent. The intermediate can be reacted with a peptide compound or an antibody herein disclosed thereby forming a conjugate compound herein disclosed that can be used for treatment of a cancer or aggressive cancer.

The expression “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “non-standard,” “unnatural” or “unusual” refers to the wide selection of non-natural, rare or synthetic amino acids such as those described by Hunt, S. in Chemisry and Biochemistry of the Amino Acids, Barrett, G. C., ed., Chapman and Hall: New York, 1985. Some examples of non-standard amino acids include non-alpha amino acids, D-amino acids.

Abbreviations used for amino acids and designation of peptides follow the rules of the IUPAC-IUB Commission of Biochemical Nomenclature in J. Biol. Chem. 1972, 247, 977-983. This document has been updated: Biochem. J., 1984, 219, 345-373; Eur. J. Biochem., 1984, 138, 9-37; 1985, 152, 1; Int. J. Pept. Prot. Res., 1984, 24, following p 84; J. Biol. Chem., 1985, 260, 14-42; Pure Appl. Chem. 1984, 56, 595-624; Amino Acids and Peptides, 1985, 16, 387-410; and in Biochemical Nomenclature and Related Documents, 2^{nd} edition, Portland Press, 1992, pp 39-67. Extensions to the rules were published in the JCBN/NC-IUB Newsletter 1985, 1986, 1989; see Biochemical Nomenclature and Related Documents, 2^{nd} edition, Portland Press, 1992, pp 68-69.

The term “antagonist” refers to a compound that reduces at least some of the effect of the endogenous ligand of a protein, receptor, enzyme, interaction, or the like.

The term “inhibitor” refers to a compound that reduces the normal activity of a protein, receptor, enzyme, interaction, or the like.

The expression “inverse agonist” refers to a compound that reduces the activity of a constitutively-active receptor below its basal level.

The term “library” refers to a collection of compounds that can be used for example for drug discovery purposes. For example, the library compounds can be peptide compounds, antibodies, peptide-conjugates and/or antibody-conjugates herein disclosed.

The term “mixture” as used herein, means a composition comprising two or more peptide-compounds or antibodies. In an embodiment a mixture is a mixture of two or more distinct peptide-compounds or antibodies. In a further embodiment, when a peptide-compound or antibody is referred to as a “mixture”, this means that it can comprise two or more “forms” of the peptide-compounds or antibodies, such as, salts, solvates, prodrugs or, where applicable, stereoisomers of the peptide-compound in any ratio. A person of skill in the art would understand that a peptide-compound or an antibody in a mixture can also exist as a mixture of forms. For example, a peptide-compound or an antibody may exist as a hydrate of a salt or as a hydrate of a salt of a prodrug of the peptide-compound or antibody. All forms of the peptide-compounds and antibodies disclosed herein are within the scope of the present application.

The term “modulator” refers to a peptide-compound or an antibody that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist.” Exemplary biological processes or mechanisms affected by a modulator include, but are not limited to, enzyme binding, receptor binding and hormone release or secretion. Exemplary chemical processes or mechanisms affected by a modulator include, but are not limited to, catalysis and hydrolysis.

The term “peptide” refers to a chemical compound comprising at least two amino acids covalently bonded together using amide bonds.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The expression “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in Protective Groups in Organic Synthesis, T. W. Greene and P. G. Wuts, eds., John Wiley & Sons, New York, 4^{th} edition, 2006, 1082 pp, ISBN 9780471697541. Examples of amino protecting groups include, but are not limited to, phthalimido, trichloroacetyl, benzyloxycarbonyl, tert butoxycarbonyl, and adamantyl-oxycarbonyl. In some embodiments, amino protecting groups are carbamate amino protecting groups, which are defined as an amino protecting group that when bound to an amino group forms a carbamate. In other embodiments, amino carbamate protecting groups are allyloxycarbonyl (Alloc), benzyloxycarbonyl (Cbz), 9 fluorenylmethoxycarbonyl (Fmoc), tert-butoxycarbonyl (Boc) and α,α dimethyl-3,5 dimethoxybenzyloxycarbonyl (Ddz). For a recent discussion of newer nitrogen protecting groups see: Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to, methyl ester, tert-butyl ester, benzyl ester, trimethylsilylethyl ester, and 2,2,2-trichloroethyl ester.

The expression “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of ref. 52, modified as in ref. 53. Such an algorithm is incorporated into the NBLAST and XBLAST programs of ref. 49. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in ref. 47. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The expression “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The expression “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry (Solid-Phase Synthesis: A Practical Guide, F. Albericio, ed., CRC Press, 2000, 848 pp, ISBN: 978-0824703592; Organic Synthesis on Solid Phase, 2^{nd} edition, Florencio Zaragoza Dörwald, Wiley-VCH, 2002, 530 pp, ISBN: 3-527-30603-X; Solid-Phase Organic Synthesis: Concepts, Strategies, and Applications, P. H. Toy, Y. Lam, eds., Wiley, 2012, 568 pp, ISBN: 978-0470599143).

The term “solid support,” “solid phase” or “resin” refers to a mechanically and chemically stable polymeric matrix utilized to conduct solid phase chemistry. This is denoted by “Resin,” “P-” or the following symbol:

[symbol]

Examples of appropriate polymer materials include, but are not limited to, polystyrene, polyethylene, polyethylene glycol (PEG, including, but not limited to, ChemMatrix® (Matrix Innovation, Quebec, Quebec, Canada; J. Comb. Chem. 2006, 8, 213-220)), polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., ed.; SPCC Ltd.: Birmingham, UK; p 205), polyacrylate (CLEAR™), polyacrylamide, polyurethane, PEGA [polyethyleneglycol poly(N,N dimethyl-acrylamide) co-polymer, Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, preferably 0.5-2%). This solid support can include as non-limiting examples aminomethyl polystyrene, hydroxymethyl polystyrene, benzhydrylamine polystyrene (BHA), methylbenzhydrylamine (MBHA) polystyrene, and other polymeric backbones containing free chemical functional groups, most typically, NH₂ or —OH, for further derivatization or reaction. The term is also meant to include “Ultraresins” with a high proportion (“loading”) of these functional groups such as those prepared from polyethyleneimines and cross-linking molecules (J. Comb. Chem. 2004, 6, 340-349). At the conclusion of the synthesis, resins are typically discarded, although they have been shown to be able to be recycled (Tetrahedron Lett. 1975, 16, 3055).

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The expression “pharmaceutically acceptable” means compatible with the treatment of subjects such as animals or humans.

The expression “pharmaceutically acceptable salt” means an acid addition salt or basic addition salt which is suitable for or compatible with the treatment of subjects such as animals or humans.

The expression “pharmaceutically acceptable acid addition salt” as used herein means any non-toxic organic or inorganic salt of any compound or antibody of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluenesulfonic and methanesulfonic acids. Either the mono or di-acid salts can be formed, and such salts may exist in either a hydrated, solvated or substantially anhydrous form. In general, the acid addition salts of the compounds or antibodies of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds or antibodies of the present disclosure, for laboratory use, or for subsequent conversion to a pharmaceutically acceptable acid addition salt.

The expression “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where CO₂H is a functional group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds, antibodies or conjugate compounds of the disclosure, for laboratory use, or for subsequent conversion to a pharmaceutically acceptable acid addition salt.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or antibody, or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound or antibody in the appropriate solvent and isolating the solvate by cooling or using an antisolvent. The solvate is typically dried or azeotroped under ambient conditions.

The term “subject” as used herein includes all members of the animal kingdom including mammals such as a mouse, a rat, a dog and a human.

The terms “suitable” and “appropriate” mean that the selection of the particular group or conditions would depend on the specific synthetic manipulation to be performed and the identity of the molecule but the selection would be well within the skill of a person trained in the art. All process steps described herein are to be conducted under conditions suitable to provide the product shown. A person skilled in the art would understand that all reaction conditions, including, for example, reaction solvent, reaction time, reaction temperature, reaction pressure, reactant ratio and whether the reaction should be performed under an anhydrous or inert atmosphere, can be varied to optimize the yield of the desired product and it is within their skill to do so.

The expression a “therapeutically effective amount”, “effective amount” or a “sufficient amount” of a compound or composition of the present disclosure is a quantity sufficient to, when administered to the subject, including a mammal, for example a human, effect beneficial or desired results, including clinical results, and, as such, a “therapeutically effective amount” or an “effective amount” depends upon the context in which it is being applied. For example, in the context of treating cancer, it is an amount of the compound, peptide compound-conjugate, antibody, antibody-conjugate or composition sufficient to achieve such treatment of the cancer as compared to the response obtained without administration of the compound, peptide compound-conjugate, antibody, antibody-conjugate or composition. The amount of a given compound, peptide compound-conjugate, antibody, antibody-conjugate or composition of the present disclosure that will correspond to an effective amount will vary depending upon various factors, such as the given drug, peptide compound-conjugate, antibody, antibody-conjugate, the pharmaceutical formulation, the route of administration, the type of disease or disorder, the identity of the subject or host being treated, and the like, but can nevertheless be routinely determined by one skilled in the art. Also, as used herein, a “therapeutically effective amount” or “effective amount” of a compound, peptide compound-conjugate, antibody, antibody-conjugate or composition of the present disclosure is an amount which inhibits, suppresses or reduces a cancer (e.g., as determined by clinical symptoms or the amount of cancerous cells) in a subject as compared to a control.

As used herein, and as well understood in the art, “treatment” or “treating” is an approach for obtaining beneficial or desired results, including clinical results. Beneficial or desired clinical results can include, but are not limited to, inhibiting vasculogenic mimicry. For example, decreasing vasculogenic mimicry tube length in a subject, a cancerous tissue and/or cells by at least about 10%, at least about 20%, at least about 30%, at least about 40%, at least about 50%, at least about 60%, at least about 70%, at least about 80% or at least about 90% greater than an untreated control subject, cancerous tissue and/or cells. For example, decreasing number of vasculogenic mimicry loops in a subject, cancerous tissue and/or cells by at least about 10%, at least about 20%, at least about 30%, at least about 40%, at least about 50%, at least about 60%, at least about 70%, at least about 80% or at least about 90% greater than an untreated control subject, cancerous tissue and/or cells. “Treatment” also means alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “tolerability” or “tolerated” as used herein means a degree to which a therapeutic agent may be endured or accepted by a subject treated with the therapeutic agent. For example, tolerability may be assessed by measuring different parameters such as (i) maintenance or absence of weight loss, (ii) duration of treatment withstood and (iii) decrease or absence of side effects. For example, it is well established that a therapeutic agent is tolerated by a subject when there is no weight loss observed during treatment using such a therapeutic agent. For example, the conjugates of the present disclosure (comprising at least one therapeutic agent) can increase the tolerability of a given therapeutic agent since the conjugate is being more selective to receptors than the therapeutic agent taken alone. Unconjugated toxins may be too toxic to be administered or used alone in a subject. Therefore, highly potent toxins can be used in antibody-drug conjugates to increase tolerability. For example, the conjugates of the present disclosure is an antibody conjugate described herein. In some embodiments, the conjugate is a conjugate antibody comprising at least one therapeutic agent herein described, for increasing the tolerability of the at least one therapeutic agent. In some embodiments, the therapeutic agent is a toxin selected from the group consisting of maytansinoids, auristatins, calicheamicins, amatoxin, and amanitin.

The term “administered” or “administering” as used herein means administration of a therapeutically effective amount of a compound, peptide compound-conjugate, antibody, antibody-conjugate or composition of the application to a cell either in vitro (e.g. a cell culture) or in vivo (e.g. in a subject).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In compositions comprising an “additional” or “second” component, the second component as used herein is chemically different from the other components or first component. A “third” component is different from the other, first, and second components, and further enumerated or “additional” components are similarly different.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

A platform allowing the transport of therapeutic agents into cancer cells for new therapies directed against primary and secondary tumours was previously developed. This approach utilizes peptide compounds derived from bacterial proteins or from ligands of receptors expressed in cancer cells (e.g. sortilins/syndecans). In the present disclosure, the conjugation of therapeutic to one of these peptide compounds for use in inhibiting vasculogenic mimicry is described. For example, phytochemicals, for example Curcumin, can be conjugated to the peptide compounds.

Disclosed herein are peptide compounds as well as conjugate compounds comprising at least one therapeutic agent connected to a peptide compound for use in inhibiting vasculogenic mimicry.

Accordingly, a first aspect is a peptide compound having at least 60% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid

and wherein at least one protecting group and/or at least one labelling agent is optionally connected to said peptide at an N- and/or C-terminal end,

for use in inhibiting vasculogenic mimicry.

Another aspect is a peptide compound having at least at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end,
for use in inhibiting vasculogenic mimicry.

Yet another aspect is a peptide compound having at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₁₉ is independently chosen from any amino acid

and wherein at least one protecting group and/or at least one labelling agent is optionally connected to said peptide at an N- and/or C-terminal end,

for use in inhibiting vasculogenic mimicry.

In some embodiments, the peptide compound targets Sortilin receptor. In some embodiments, the peptide compound is for use in targeting Sortilin receptor.

For example, the peptide compound is a peptide compound that comprises:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

For example, the peptide compound is a peptide compound that consists essentially of:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

For example, the peptide compound is a peptide compound that consists of:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

According to another aspect, there is provided a peptide compound that comprises a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X&, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid

and wherein at least one protecting group and/or at least one labelling agent is optionally connected to said peptide at an N- and/or C-terminal end,

for use in inhibiting vasculogenic mimicry.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound chosen from peptide compounds of formula (I), formula (II), formula (III), formula (IV), formula (V), formula (VI), formula (VII), formula (VIII), formula (IX), formula (X), formula (X), formula (XII) and formula (XIII).

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (I) or SEQ ID NO: 1.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (II) or SEQ ID NO: 2.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (III) or SEQ ID NO: 3.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IV) or SEQ ID NO: 4.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (V) or SEQ ID NO: 5.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VI) or SEQ ID NO: 6.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VII) or SEQ ID NO: 7.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VIII) or SEQ ID NO: 8.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IX) or SEQ ID NO: 9.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (X) or SEQ ID NO: 10.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XI) or SEQ ID NO: 11.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XII) or SEQ ID NO: 12.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XIII) or SEQ ID NO: 13.

For example, the peptide compound has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (LI) or SEQ ID NO: 23.

In one embodiment, n is 0. In one embodiment, n is 1. In one embodiment, n is 2. In one embodiment, n is 3. In one embodiment, n is 4. In one embodiment, n is 5.

In an embodiment, the peptide compound is represented by formula (I) or formula (II). In one embodiment, the peptide compound is represented by formula (I) or SEQ ID NO: 1. In one embodiment, the peptide compound is represented by formula (II) or SEQ ID NO: 2. In one embodiment, the peptide compound is represented by formula (III) or formula (IV). In one embodiment, the peptide compound is represented by formula (III). In one embodiment, the peptide compound is represented by formula (IV). In an embodiment, the peptide compound is represented by formula (V), formula (VI), formula (VII), formula (VIII), formula (IX) or formula (X). In one embodiment, the peptide compound is represented by formula (V). In one embodiment, the peptide compound is represented by formula (VI). In one embodiment, the peptide compound is represented by formula (Vii). In one embodiment, the peptide compound is represented by formula (VIII). In one embodiment, the peptide compound is represented by formula (IX). In one embodiment, the peptide compound is represented by formula (X). In one embodiment, the peptide compound is represented by formula (X), formula (XII) or formula (XIII). In one embodiment, the peptide compound is represented by formula (XI). In one embodiment, the peptide compound is represented by formula (XII). In one embodiment, the peptide compound is represented by formula (XIII). In one embodiment, the peptide compound is represented by formula (LI).

In one embodiment, the peptide compound is represented by the amino acid sequence of SEQ ID NO: 1. In one embodiment, the peptide compound is represented by the amino acid sequence of SEQ ID NO: 2. In one embodiment, the peptide compound is represented by the amino acid sequence of SEQ ID NO: 3. In one embodiment, the peptide compound is represented by the amino acid sequence of SEQ ID NO: 4. In one embodiment, the peptide compound is represented by the amino acid sequence of SEQ ID NO: 5. In one embodiment, the peptide compound is represented by the amino acid sequence of SEQ ID NO: 6. In one embodiment, the peptide compound is represented by the amino acid sequence of SEQ ID NO: 7. In one embodiment, the peptide compound is represented by the amino acid sequence of SEQ ID NO: 8. In one embodiment, the peptide compound is represented by the amino acid sequence of SEQ ID NO: 9. In one embodiment, the peptide compound is represented by the amino acid sequence of SEQ ID NO: 10. In one embodiment, the peptide compound is represented by the amino acid sequence of SEQ ID NO: 11. In one embodiment, the peptide compound is represented by the amino acid sequence of SEQ ID NO: 12. In one embodiment, the peptide compound is represented by the amino acid sequence of SEQ ID NO: 13. In one embodiment, the peptide compound is represented by the amino acid sequence of SEQ ID NO: 23.

In one embodiment, at least one protecting group is connected to said peptide at an N- and/or C-terminal end.

In one embodiment, a succinyl group is connected to the peptide compound. For example, the peptide compound has the sequence of Succinyl-IKLSGGVQAKAGVINMFKSESY, corresponding to SEQ ID NO: 6 and having a succinyl group attached thereto at the N-terminal end.

In one embodiment, an acetyl group is connected to the peptide compound. For example, the peptide compound has the sequence of Acetyl-GVRAKAGVRNMFKSESY (SEQ ID NO: 14). For example, the peptide compound has the sequence of Acetyl-GVRAKAGVRN(Nle)FKSESY (SEQ ID NO: 15). For example, the peptide compound has the sequence of Acetyl-YKSLRRKAPRWDAPLRDPALRQLL (SEQ ID NO: 16). For example, the peptide compound has the sequence of Acetyl-YKSLRRKAPRWDAYLRDPALRQLL (SEQ ID NO: 17). For example, the peptide compound has the sequence of Acetyl-YKSLRRKAPRWDAYLRDPALRPLL (SEQ ID NO: 18).

In one embodiment, at least one labelling agent is connected to said peptide at an N- and/or C-terminal end.

The person skilled in the art will understand that commonly used labelling agents can be used. For example, the labelling agent is a vitamin. For example, the labelling agent is biotin. For example, the labelling agent is used as a fluorescent probe and/or as an imaging agent.

Z In one embodiment, the peptide compound is biotinylated. For example, the peptide compound has the sequence of IKLSGGVQAKAGVINMFKSESYK(Biotin), corresponding to SEQ ID NO: 7 and having a biotin molecule attached thereto at the C-terminal end.

For example, the peptide compound is represented by Formula (XXXVI):

Succinyl-IKLSGGVQAKAGVINMFKSESY (XXXVI)
that comprises the peptide compound having SEQ ID NO: 6 wherein a succinyl group is attached at the N-terminal end.

In one embodiment, X₁₆ is independently chosen from Q, P, Y, I and L.

For example, X₁₆ is Q. For example, X₁₆ is P. For example, X₁₆ is Y. For example, X₁₆ is I.

In one embodiment, X₁₇ is independently chosen from Q, P, Y, I and L.

For example, X₁₇ is Q. For example, X₁₇ is P. For example, X₁₇ is Y. For example, X₁₇ is I.

In one embodiment, X₂₀ is independently chosen from Q, P, Y, I and L.

For example, X₂₀ is Q. For example, X₂₀ is P. For example, X₂₀ is Y. For example, X₂₀ is I.

In one embodiment, X₂₁ is independently chosen from Q, P, Y, I and L.

For example, X₂₁ is Q. For example, X₂₁ is P. For example, X₂₁ is Y. For example, X₂₁ is I.

In one embodiment, the peptide compound is chosen from:

(SEQ ID NO: 1)
X₁X₂X₃X₄X₅GVX₆AKAGVX₇NX₈FKSESY;
(SEQ ID NO: 2)
(X₉)ₙGVX₁₀AKAGVX₁₁NX₁₂FKSESY;
(SEQ ID NO: 3)
YKX₁₃LRRX₁₄APRWDX₁₅PLRDPALRX₁₆X₁₇L;
(SEQ ID NO: 4)
YKX₁₈LRR(X₁₉)ₙPLRDPALRX₂₀X₂₁L;
(SEQ ID NO: 5)
IKLSGGVQAKAGVINMDKSESM;
Succinyl-IKLSGGVQAKAGVINMFKSESY (that comprises
SEQ ID NO: 6 wherein a succinyl group is attached
thereto at the N-terminal end);
IKLSGGVQAKAGVINMFKSESYK(Biotin) (that comprises
SEQ ID NO: 7 wherein a biotin molecule is attached
thereto at the C-terminal end);
(SEQ ID NO: 8)
GVQAKAGVINMFKSESY;
(SEQ ID NO: 14)
Acetyl-GVRAKAGVRNMFKSESY;
(SEQ ID NO: 15)
Acetyl-GVRAKAGVRN(Nle)FKSESY;
(SEQ ID NO: 16)
Acetyl-YKSLRRKAPRWDAPLRDPALRQLL;
(SEQ ID NO: 17)
Acetyl-YKSLRRKAPRWDAYLRDPALRQLL;
(SEQ ID NO: 18)
Acetyl-YKSLRRKAPRWDAYLRDPALRPLL;
(SEQ ID NO: 23)
GVRAKAGVRN(Nle)FKSESYC;
and
(SEQ ID NO: 24)
Acetyl-GVRAKAGVRN(Nle)FKSESYC.

In one embodiment, the peptide compounds can be modified at the C- and/or N-terminal by the addition of one or more amino acid residue in order to obtain or increase preferential binding sites at the peptide terminal end. For example, the amino acid can be cysteine. For example, the amino acid can be lysine. For example, the amino acid can be cysteine added at the C-terminal of a peptide. In one embodiment, the peptide compound is modified by the addition of cysteine at the C-terminal. In a specific embodiment, the peptide compound has the sequence of Acetyl-GVRAKAGVRN(Nle)FKSESY corresponding to SEQ ID NO: 15 is modified by the addition of cysteine at the C-terminal.

In an aspect, the peptide compound or derivative thereof described herein specifically binds to a polypeptide having the amino acid sequence as shown in any one of SEQ ID NOs: 25-50, an analog thereof, or a fragment thereof, for use in inhibiting vasculogenic mimicry. In another aspect, the peptide compound or derivative thereof described herein targets Sortilin receptor. In another aspect, the peptide compound or derivative thereof described herein is for use in targeting Sortilin receptor. In another aspect, the peptide compound or derivative thereof described herein binds at least 2, 3, 4 or 5 contiguous amino acid residues as shown in any one of SEQ ID NOs: 25-50, an analog thereof, or a fragment thereof. For example, the peptide compound or derivative thereof binds at least 2 contiguous amino acid residues as shown in SEQ ID NO: 25. For example, the peptide compound or derivative thereof binds at least 3 contiguous amino acid residues as shown in SEQ ID NO: 25. For example, the peptide compound or derivative thereof binds at least 4 contiguous amino acid residues as shown in SEQ ID NO: 25. For example, the peptide compound or derivative thereof binds at least 5 contiguous amino acid residues as shown in SEQ ID NO: 25.

The peptide compounds described herein can be connected, linked, mixed or conjugated to small molecules, peptides, proteins, oligonucleotides, diagnostic agents, imaging or radionuclide agents, large molecules such as monoclonal antibodies, therapeutic agents such phytochemicals or to drug delivery systems including nanoparticles, liposomes, nanotubes, graphene particles loaded with a therapeutic agent, imaging agent, gene, siRNA. The resulting conjugate compounds can be used as mono- or combined therapies for example for inhibiting vasculogenic mimicry.

Accordingly, another aspect disclosed herein is a conjugate compound having the formula of A-(B)ₙ,

wherein
n is 1, 2, 3 or 4;
A is a peptide compound as defined herein, wherein said peptide is optionally protected by a protecting group; and
B is at least one therapeutic agent, wherein B is connected to A,
optionally the peptide compound is cyclic,
for use in inhibiting vasculogenic mimicry.

Yet another aspect disclosed herein is a conjugate compound having the formula of A-(B)ₙ,

wherein
n is 1, 2, 3 or 4;
A is a peptide compound as defined in the present disclosure, wherein said peptide compound is optionally protected by a protecting group; and
B is at least one therapeutic agent, wherein B is connected to A, optionally at a free amine of said peptide compound, at an N-terminal position of said peptide compound, at a free —SH of said peptide compound, or at a free carboxyl of said peptide compound,
optionally the peptide compound is cyclic,
for use in inhibiting vasculogenic mimicry.

In some embodiments, the conjugate peptide described herein targets Sortilin receptor. In some embodiments, the conjugate peptide described herein is for use in targeting Sortilin receptor.

Yet another aspect disclosed herein is a conjugate compound having the formula of A-(B)ₙ,

wherein
n is 1, 2, 3 or 4;
A is a peptide compound as defined herein; and
B is at least one therapeutic agent, wherein B is connected to A at a free amine of a lysine residue of said peptide compound, optionally via a linker, or at an N-terminal position of said peptide compound, optionally via a linker,
optionally the peptide compound is cyclic,
for use in treating cancer or aggressive cancer.

In an embodiment, B is connected to A via a linker, optionally a cleavable linker.

For example, the at least one therapeutic agent is a phytochemical chosen from curcumin, omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

In an embodiment, the therapeutic agent is a phytochemical or an anticancer agent.

In an embodiment, the phytochemical is curcumin.

In an embodiment, the conjugate compound is chosen from:

GVRAK(curcumin)AGVRN(Nle)FK(curcumin)SESY Formula (XIV)
that comprises the peptide compound having SEQ ID NO: 10 wherein each lysine residue has a curcumin molecule connected thereto; and

YK(curcumin)SLRRK(curcumin)APRWDAPLRDPALRQLL Formula (XV)
that comprises the peptide compound having SEQ ID NO: 11 wherein each lysine residue has a curcumin molecule connected thereto.

For example, the conjugate compound is represented by formula (XIV).

For example, the conjugate compound is represented by formula (XV).

In an embodiment, the conjugate compound is chosen from:

Acetyl-GVRAK(curcumin)AGVRN(Nle)FK(curcumin)SESY Formula (XVI)
that comprises the peptide compound having SEQ ID NO: 15 wherein each lysine residue has a curcumin molecule connected thereto, and

Acetyl-YK(curcumin)SLRRK(curcumin)APRWDAPLRDPALRQLL Formula (XVII)
that comprises the peptide compound having SEQ ID NO: 16 wherein each lysine residue has a curcumin molecule connected thereto.

For example, the conjugate compound is represented by formula (XVI).

For example, the conjugate compound is represented by formula (XVII).

In an embodiment, the therapeutic agent is an anticancer agent.

In an embodiment, the anticancer agent is docetaxel.

In an embodiment, the conjugate compound is represented by formula (XIX):

GVRAK(docetaxel)AGVRN(Nle)FK(docetaxel)SESY Formula (XIX)
that comprises the peptide compound having SEQ ID NO: 10 wherein each lysine residue has a docetaxel molecule connected thereto.

In another embodiment, the conjugate compound is represented by formula (XXIII):

Acetyl-GVRAK(docetaxel)AGVRN(Nle)FK(docetaxel)SESY Formula (XXIII)
that comprises the peptide compound having SEQ ID NO: 15 wherein each lysine residue has a docetaxel molecule connected thereto.

In an embodiment, the anticancer agent is doxorubicin.

In an embodiment, the conjugate compound is represented by formula (XXVI):

GVRAK(doxorubicin)AGVRN(Nle)FK(doxorubicin)SESY Formula (XXVI)
that comprises the peptide compound having SEQ ID NO: 10 wherein each lysine residue has a doxorubicin molecule connected thereto.

In another embodiment, the conjugate compound is represented by formula (XXVIII):

Acetyl-GVRAK(doxorubicin)AGVRN(Nle)FK(doxorubicin)SESY Formula (XXVIII)
that comprises the peptide compound having SEQ ID NO: 15 wherein each lysine residue has a doxorubicin molecule connected thereto.

In an embodiment, the anticancer agent is cabazitaxel.

In an embodiment, the anticancer agent is aldoxorubicin.

In an embodiment, the conjugate compound is represented by formula (LI):

GVRAKAGVRN(Nle)FKSESYC(aldoxorubicin) Formula (LI)
that comprises the peptide compound having SEQ ID NO: 23 wherein cysteine residue has an aldoxorubicin molecule connected thereto, or
that comprises the peptide compound having SEQ ID NO: 10 wherein a cysteine residue is added to C-terminal of said peptide compound, and wherein the cysteine residue has an aldoxorubicin molecule connected thereto.

In an embodiment, the conjugate compound is represented by formula (LII):

Acetyl-GVRAKAGVRN(Nle)FKSESYC(aldoxorubicin) Formula (LII)
that comprises the peptide compound having SEQ ID NO: 24 wherein cysteine residue has an aldoxorubicin molecule connected thereto, or
that comprises the peptide compound having SEQ ID NO: 15 wherein a cysteine residue is added to C-terminal of said peptide compound, and wherein the cysteine residue has an aldoxorubicin molecule connected thereto.

In an embodiment, B, the at least one therapeutic agent, is connected to A, the peptide compound, at said free amine of said lysine residue of said peptide compound, via a linker.

In an embodiment, B, the at least one therapeutic agent, is connected to A, the peptide compound, at said N-terminal position of said peptide compound, via a linker.

In an embodiment, the linker is chosen from succinic acid and dimethyl glutaric acid linker.

For example, the linker is a cleavable linker. For example, the linker is a non-cleavable linker.

For example, the conjugate compound can comprise a cleavable linker connected the at least one therapeutic agent to the peptide compound or the antibody. For example, the at least one therapeutic agent can be released from the peptide compound or antibody by the action of esterases on the ester bond.

For example, a therapeutic agent can be conjugated to the peptide compound or antibody on free amines available on the peptide or antibody, at the lysine or amino-terminal, by forming a bond such as a peptide bond.

In an embodiment, the isolated antibody binds at least 1, 2, 3, 4, 5, 6, 7, 8, 9, 10, 11, 12, 13, 14, 15, 16, 17, 18, 19, or 20 contiguous residues of any one of SEQ ID NOs: 25-50. In an embodiment, the isolated antibody binds at least 2 contiguous residues of any one of SEQ ID NOs: 25-50. In an embodiment, the isolated antibody binds at least 3 contiguous residues of any one of SEQ ID NOs: 25-50. In an embodiment, the isolated antibody binds at least 4 contiguous residues of any one of SEQ ID NOs: 25-50. In an embodiment, the isolated antibody binds at least 5 contiguous residues of any one of SEQ ID NOs: 25-50. In an embodiment, the isolated antibody binds at least 6 contiguous residues any one of SEQ ID NOs: 25-50. In an embodiment, the isolated antibody binds at least 7 contiguous residues any one of SEQ ID NOs: 25-50. In an embodiment, the isolated antibody binds at least 8 contiguous residues any one of SEQ ID NOs: 25-50. In an embodiment, the isolated antibody binds at least 9 contiguous residues any one of SEQ ID NOs: 25-50. In an embodiment, the isolated antibody binds at least 10 contiguous residues any one of SEQ ID NOs: 25-50. In an embodiment, the isolated antibody binds at least 11 contiguous residues any one of SEQ ID NOs: 25-50. In an embodiment, the isolated antibody binds at least 12 contiguous residues any one of SEQ ID NOs: 25-50. In an embodiment, the isolated antibody binds at least 13 contiguous residues any one of SEQ ID NOs: 25-50. In an embodiment, the isolated antibody binds at least 14 contiguous residues any one of SEQ ID NOs: 25-50. In an embodiment, the isolated antibody binds at least 15 contiguous residues any one of SEQ ID NOs: 25-50. In an embodiment, the isolated antibody binds at least 16 contiguous any one of SEQ ID NOs: 25-50. In an embodiment, the isolated antibody binds at least 17 contiguous residues any one of SEQ ID NOs: 25-50. In an embodiment, the isolated antibody binds at least 18 contiguous residues any one of SEQ ID NOs: 25-50. In an embodiment, the isolated antibody binds at least 19 contiguous residues any one of SEQ ID NOs: 25-50. In an embodiment, the isolated antibody binds at least 20 contiguous any one of SEQ ID NOs: 25-50.

In an embodiment, the isolated antibody is monoclonal, polyclonal, chimeric or humanized. In an embodiment, the isolated antibody is an antibody fragment. In an embodiment, the antibody fragment is a Fab, Fab′, F(ab′)2, scFv, dsFv, ds-scFv, dimers, minibodies, diabodies, or multimers thereof or bispecific antibody fragments. In an embodiment, the isolated antibody is a monoclonal antibody. In an embodiment, the mAb is anti-Sortilin mAb #1 or anti-Sortilin mAb #2. In an embodiment, the mAb is anti-Sortilin mAb #1. In an embodiment, the mAb is anti-Sortilin mAb #2.

A person skilled in the art will understand that other known antibodies or binding fragments thereof that specifically bind Sortilin may be used. For example, Anti-Sortilin antibody ab6640 (manufactured by abcam) which binds amino acid residues 800 to the c-terminus of Sortilin may be used.

Non-limiting examples of antibodies or fragments thereof binding Sortilin that may be used include: Cat #PA5-77535, Cat #OSS00052W, Cat #MA5-31438, Cat #OSS00011W, Cat #PA1-18312, Cat #PA5-29195, Cat #PA5-96865, Cat #703207, Cat #PA5-47462, Cat #PA5-19481, Cat #OSS00010W, Cat #MA5-31437, Cat #OSS00041G (manufactured by Invitrogen); Cat #ab16640, Cat #ab188586 (manufactured by abcam); Cat #AF3154, Cat #AF2934, Cat #MAB3154, Cat #BAF3154, Cat #BAF2934, Cat #FAB3154 (manufactured by R&D Systems); clone W16078A (manufactured by BioLegend); Cat #A56294, Cat #A56295, Cat #A56296 (manufactured by Epigentek); clone CL6526, clone CL6528, HPA006889 (manufactured by Atlas Antibodies); Cat #12369-1-AP (manufactured by Proteintech); sc-376561, sc-376576, sc-376561 HRP, sc-376561 AC (manufactured by Santa Cruz Biotechnology); Cat #N2177-52A, Cat #N2177-51A, Cat #N2177-52, Cat #133710, Cat #133710-HRP, Cat #133710-Biotin, Cat #133710-FITC (manufactured by United States Biological); Cat #A01666 (manufactured by Boster Biological Technology); Cat #LS-C198140, Cat #LS-C672508, Cat #LS-C672507, Cat #LS-C672506, Cat #LS-C672509, Cat #LS-C37627, Cat #LS-C37628, Cat #LS-C73437, Cat #LS-C94842, Cat #LS-C94818, Cat #LS-C94912, Cat #LS-C94806, Cat #LS-C668404 (manufactured by Lifespan biosciences); Cat #A7926, Cat #A4101 (manufactured by ABclonal Technology); Cat #NBP2-76498, Cat #NBP2-76501, Cat #NBP2-89745, Cat #H00006172-M01 (manufactured by Novus Biologicals); Cat #orb525096, Cat #orb525097, Cat #orb331290, Cat #orb243645, Cat #orb243644, Cat #orb243646, Cat #orb243643, Cat #orb255650, Cat #orb412666, Cat #orb416271, Cat #orb416270, Cat #orb416272, Cat #orb416269, Cat #orb446447, Cat #orb488236, Cat #orb101927, Cat #orb373861, Cat #orb103522, Cat #orb484107 (manufactured by Biorbyt); Cat #H00006272-M01, Cat #H00006272-A01 (manufactured by Abnova); Cat #DPAB-DC2767, Cat #CABT-B1343 (manufactured by Creative Diagnostics Incorporation); Cat #OAGA03665, Cat #OAAN03744, Cat #ARP64630_P050, Cat #ARP87664_P050, Cat #ARP81139_P050, Cat #OACD07428, Cat #OACA03712 (manufactured by Aviva Systems Biology); Cat #TA351744, Cat #TA813064, Cat #CF813064, Cat #TA328904 (manufactured by Origene); Cat #R-150-100 (manufactured by Biosensis); Cat #AB9712 (manufactured by Millipore); Cat #67531 (manufactured by NovaTeinBio), and Cat #612100, Cat #612101 (manufactured by BD Biosciences), all of which are presently incorporated herein in their entirety.

In an aspect, also provided is a method of making an isolated antibody described herein, in which the isolated antibody specifically binds an isolated polypeptide comprising the sequence as shown in any one of SEQ ID NOs: 25-50, an analog thereof, or a fragment thereof, by i) immunizing an animal with an immunogenic form of the isolated polypeptide; ii) screening an expression library; or iii) using phage display.

In an aspect, also provided is a conjugate antibody having the formula of A′-(B)ₙ, wherein
n is 1, 2, 3, 4, 5, 6, 7, 8, 9, 10, 11, or 12;
A′ is an isolated antibody as described herein, wherein said isolated antibody is optionally protected by a protecting group; and
B is at least one therapeutic agent, wherein B is connected to A′, optionally at a free amine of said isolated antibody, at an N-terminal position of said isolated antibody, at a free —SH of said isolated antibody, or at a free carboxyl of said isolated antibody,
for use in inhibiting vasculogenic mimicry and/or treating a cancer.

In a further aspect, also provided is A conjugate antibody having the formula of A′-(B)ₙ,
wherein
n is 1, 2, 3, 4, 5, 6, 7, 8, 9, 10, 11, or 12;
A′ is an isolated antibody as described herein, wherein said isolated antibody is optionally protected by a protecting group; and
B is at least one therapeutic agent, wherein B is connected to A′ at a free amine of a lysine residue of said isolated antibody, optionally via a linker, or at an N-terminal position of said isolated antibody, optionally via a linker,
for use in inhibiting vasculogenic mimicry and/or treating a cancer.

In an embodiment, the conjugate antibody targets Sortilin receptor. In an embodiment, the conjugate antibody is for use in targeting Sortilin receptor.

In an embodiment, wherein B is connected to A′ via a linker, optionally a cleavable linker or a non-cleavable linker. In an embodiment, the at least one therapeutic agent is an anticancer agent. In an embodiment, the anticancer agent docetaxel, doxorubicin, cabazitaxel, aldoxorubicin, maytansinoids, auristatins, calicheamicins, amatoxins, amanitin and oligopeptidomimetics (e.g. tubulysins). In an embodiment, the at least one therapeutic agent is a phytochemical, optionally curcumin. In an embodiment, the anticancer agent is docetaxel. In an embodiment, the anticancer agent is doxorubicin. In an embodiment, the anticancer agent is cabazitaxel. In an embodiment, the anticancer agent is aldoxorubicin.

The number of therapeutic agent in an antibody conjugate can be heterogeneous. For example, for Trastuzumab emtansine, it varies between 0 to 8 molecules of maytansine per molecule of antibody for an average of 3.5 to 4 molecules of maytsansine per antibody.

In an embodiment, the conjugate compound or antibody conjugate comprises 1 molecule of the therapeutic agent connected to the peptide compound or the isolated antibody. In an embodiment, the conjugate compound or antibody conjugate comprises at least 1 molecule of the therapeutic agent connected to the peptide compound or the isolated antibody. In an embodiment, the conjugate compound or antibody conjugate comprises up to 8 molecules of the therapeutic agent connected to the peptide compound or the isolated antibody. In an embodiment, the antibody conjugate comprises up to 10 molecules of the therapeutic agent connected to the isolated antibody. In an embodiment, the antibody conjugate comprises up to 12 molecules of the therapeutic agent connected to the isolated antibody.

In an embodiment, the conjugate compound or antibody conjugate comprises 2 molecules of the therapeutic agent connected to the peptide compound or the isolated antibody. In an embodiment, the conjugate compound or antibody conjugate comprises 3 molecules of the therapeutic agent connected to the peptide compound or the isolated antibody. In an embodiment, the conjugate compound or antibody conjugate comprises 4 molecules of the therapeutic agent connected to the peptide compound or the isolated antibody. In an embodiment, the conjugate compound or antibody conjugate comprises 1-8 molecules of the therapeutic agent connected to the peptide compound or the isolated antibody. In an embodiment, the antibody conjugate comprises 0-12, optionally 0-10, optionally 0-8 molecules of the therapeutic agent connected to the isolated antibody. In another embodiment, the antibody conjugate comprises more than zero and up to 12, optionally 10, optionally 8 molecules of the therapeutic agent connected to the isolated antibody. In another embodiment, the antibody conjugate comprises 1-12, optionally 1-10, optionally 1-8 molecules of the therapeutic agent connected to the isolated antibody.

In an embodiment, the conjugate antibody comprises a therapeutic agent selected from the group consisting of a cytotoxic agent, a toxin, an anticancer peptide, a targeted drug, an immunotherapy, a phytochemical, and/or oligopeptidomimetic. In another embodiment, the therapeutic agent is an Alkylating agent; a Platinum coordination agent selected form the group consisting of Cisplatin, Carboplatin, and Oxaliplatin; Antimetabolites; a Microtubule damaging agents selected from the group consisting of Vincristine, Vinblastine, Vinorelbine, Paclitaxel, and Docetaxel; a Topoisomerase-2 inhibitor such as Etoposide; a Topoisomerase-1 inhibitor selected from the group consisting of Topotecan and Irinotecan; an Antibiotics selected from the group consisting of Actinomycin D, Doxorubicin, Daunorubicin, Epirubicin, Bleomcins, and Mitomycin C; Hydroxyurea; L-Asparaginase; Tretinoin; Maytansinoids; Auristatins; Calicheamicins; Amatoxin; Amanitin; D-Peptide A; D-Peptide B; D-Peptide C; D-Peptide D; D-K619; NRC-03; NRC-07; Gomesin; Hepcidin Th2-3; Dermaseptin B2; PTP7; MGA2; HNP-1; Tachyplesin; Temporin-1 Cea; NK-2; Cecropin Cb1; a Tyrosine protein kinase inhibitors selected from the group consisting of Imatinib and Dasatinib; an EFG receptor inhibitor selected from the group consisting of Gefitnib and Erlotinib; an Angiogenesis inhibitor selected from the group consisting of Bevacizumab, Thalidomide, Endostatin, Angiostatin, Angiopoitein, and Cannabionoids; a Proteasome inhibitor selected from the group consisting of Bortezomib, Carfizomib, Izazomib, Marizomib, and Epoxomicin; a mAb selected from the group consisting of Rituximab and Trastuzumab; a Check point inhibitor; CAR-T cell therapy; an Antibody; an Antibody drug conjugate; a Bispecific T cell engagers and Bispecific antibodies; a Genetically Engineered T cell-mediated cell killing; an Oncolytic virus; a T-cell mediated cytolysis; an Alkaloid selected from the group consisting of Chlorogenic acid, Theobromine, and Theophylline; an Anthocyanin selected from the group consisting of Cyanidin and Mavdin; a Carotenoid selected from the group consisting of Beta-Carotene, Lutein, and Lycopene; a Coumestan; a Flavan-3-Ol; a Flavonoid selected from the group consisting of Epicatechin, Hesperidin, Isorhamnetin, Kaempferol, Myricetin, Naringin, Nobiletin, Proanthocyanidins, Quercetin, Rutin, and Tangeretin; a Hydroxycinnamic Acid selected from the group consisting of Chicoric acid, Coumarin, Ferulic acid, and Scopoletin; an Isoflavone selected from the group consisting of Daidzein and Genistein; a Lignan such as Silymarin; a Monoterpene selected from the group consisting of Geraniol and Limonene; an Organosulfide selected from the group consisting of Allicin, Glutathione, Indole-3-Carbinol, Isothiocyanates, and Sulforaphane; Damnacanthal; Digoxin; Phytic acid; a Phenolic Acid selected from the group consisting of Capsaicin, Ellagic Acid, Gallic acid, Rosmarinic acid, and Tannic Acid; a Phytosterol such as Beta-Sitosterol; a Saponin; a Stylbene selected from the group consisting of Pterostilbene and Resveratrol; a Triterpenoid such as Ursolic acid; an Xanthophyll selected from the group consisting of Astaxanthin and Beta-Cryptoxanthin; a Monophenol such as Hydroxytyrosol; or Tubulysins.

For example, the treating cancer or aggressive cancer comprises inhibiting vasculogenic mimicry in cells expressing Sortilin.

In an aspect, the compound, isolated antibody or antibody conjugate described herein is for use in inhibiting vasculogenic mimicry. In an aspect, the compound, isolated antibody or antibody conjugate described herein targets Sortilin receptor. In an aspect, the compound, isolated antibody or antibody conjugate described herein is for use in targeting Sortilin receptor.

In a further aspect, the compound, isolated antibody or antibody conjugate described herein is for use in inhibiting vasculogenic mimicry and/or treating cancer in a CD133 positive cell.

In an embodiment, the compound, isolated antibody or antibody conjugate decreases vasculogenic mimicry tube length in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an embodiment, the compound, isolated antibody or antibody conjugate decreases number of vasculogenic mimicry loops in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In an embodiment, the compound, isolated antibody or antibody conjugate decreases vasculogenic mimicry tube length in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In an embodiment, the compound, isolated antibody or antibody conjugate decreases number of vasculogenic mimicry loops in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In an aspect, the compound, isolated antibody or antibody conjugate described herein is for treatment of cancer or aggressive cancer.

For example, the treating cancer or aggressive cancer comprises decreasing vasculogenic mimicry tube length in a subject, a cancerous tissue and/or cells expressing Sortilin by at least about 10%, at least about 20%, at least about 30%, at least about 40%, at least about 50%, at least about 60%, at least about 70%, at least about 80% or at least about 90% greater than an untreated control subject, cancerous tissue and/or cells expressing Sortilin.

For example, the treating cancer or aggressive cancer comprises decreasing number of vasculogenic mimicry loops in a subject, a cancerous tissue and/or cells expressing Sortilin by at least about 10%, at least about 20%, at least about 30%, at least about 40%, at least about 50%, at least about 60%, at least about 70%, at least about 80% or at least about 90% greater than an untreated control subject, cancerous tissue and/or cells expressing Sortilin.

For example, the treating cancer or aggressive cancer comprises decreasing vasculogenic mimicry tube length in a subject, a cancerous tissue and/or cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

For example, the treating cancer or aggressive cancer comprises decreasing vasculogenic mimicry loops in a subject, a cancerous tissue and/or cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

For example, the cells expressing Sortilin are immune cells, optionally macrophages, CD4+, CD8+, B220+, bone marrow-derived cells basophils, eosinophils and cytotoxic T lymphocytes, Natural Killer (NK) cells, T helper type 1 (Th1) cells.

For example, the cells expressing Sortilin are cancer cells, optionally ovarian cancer cells, endometrial cancer cells, breast cancer cells (e.g. triple negative breast cancer cells), prostate cancer cells, colorectal cancer cells, lung cancer cells, pancreas cancer cells, skin cancer cells, brain (gliomas) cancer cells, urothelial cancer cells, carcinoid cancer cells, renal cancer cells, testis cancer cells, pituitary cancer cells and blood cancer cells such as bone marrow cancer cells, diffuse large B cell lymphoma cancer cells, myeloma cancer cells or chronic B cell leukemia cancer cells.

For example, the cells presenting vasculogenic mimicry are cancer cells, optionally breast cancer cells (e.g. triple negative breast cancer cells), gliomas cells, hepatocellular carcinoma cells, colorectal cancer cells, medulloblastoma cells, bi-directional differentiated malignant tumour cells, gastric cancer cells, prostate cancer cells, sarcomas cells, gallbladder cancer cells, oral/laryngeal squamous cell carcinoma cells, melanoma cells, non-small cell lung cancer cells or ovarian cancer cells.

For example, the triple negative breast cancer cells are HCC1599, HCC1937, HCC1143, MDA-MB468, HCC38, HCC70, HCC1806, HCC1187, DU4475, BT-549, Hs578T, MDA-MB231, MDA-MB436, MDA-MB157, MDA-MB453, BT-20, or HCC1395 cells.

For example, the cells are CD133 positive cells.

Conjugate compound or antibody conjugate herein disclosed can also be used to transport therapeutic agents into the cell as they are not a substrate of efflux pumps such as the P-glycoprotein membrane transporter pump which pumps out other therapeutic agents from multi resistant drug cells.

In an aspect, there is provided a method for obtaining the peptide compound, comprising i) providing a library of binder peptides; and ii) selecting sortilin-binding peptides from the library by means of affinity selection using a target;

wherein the target is immobilized on a solid support;

wherein the target is comprised of amino acid sequence as shown in any one of SEQ ID NOs: 25-50, an analog thereof, or a fragment thereof; and

wherein the target is in interaction with the sortilin-binding peptide.

In a further aspect, there is provided a process for preparing conjugate compound or antibody conjugate herein disclosed, the process comprising:
reacting a linker together with said therapeutic agent so as to obtain an intermediate;
optionally purifying said intermediate;
reacting said intermediate together with peptide compound so as to obtain said conjugate compound, or reacting said intermediate together with isolated antibody so as to obtain said antibody conjugate; and
optionally purifying said conjugate compound or antibody conjugate;
wherein the therapeutic agent is connected to the peptide compound or the isolated antibody at a free amine of a lysine residue or an N-terminal; and wherein the peptide compound comprises 1, 2, 3 or 4 therapeutic agent molecules connected thereto, wherein the isolated antibody comprises 1-12, optionally 1-10, optionally 1-8 therapeutic agent molecules connected thereto.

For example, the peptide compound or the isolated antibody comprises 1 therapeutic agent molecule connected thereto. For example, the peptide compound or the isolated antibody comprises 2 therapeutic agent molecules connected thereto. For example, the peptide compound or the isolated antibody comprises 3 therapeutic agent molecules connected thereto. For example, the peptide compound or the isolated antibody comprises 4 therapeutic agent molecules connected thereto.

For example, the linker is succinic acid. For example, the linker is a dimethyl glutaric acid linker.

In an embodiment, the peptide compound or the isolated antibody is protected at said N-terminal prior to reacting with said intermediate.

For example, a protecting group such as FMOC can be added as a protecting group to a free amine on the therapeutic agent prior to incorporation with a linker. After its synthesis, the conjugate compound or the antibody conjugate can undergo deprotection from the protecting group. For example, the conjugate compound or the antibody conjugate comprising the protecting agent FMOC can be deprotected using piperidin. The person skilled in the art would readily understand that other known chemical reagents may be used for deprotection of conjugate compounds or the antibody conjugates.

For example, the N-terminal of the therapeutic agent, the peptide compound and/or the antibody can be capped by its acetylation, thereby providing a non-reversible protecting group at the N-terminal.

In an embodiment, the intermediate is activated prior to reacting with said peptide compound or said antibody.

For example, the intermediate is activated prior to reacting with said compound with a coupling agent, optionally chosen from N,N,N′,N′-Tetramethyl-O-(benzotriazol-1-yl)uronium tetrafluoroborate (TBTU), (2-(1H-benzotriazol-1-yl)-1,1,3,3-tetramethyluronium hexafluorophosphate) (HBTU), and (1-[Bis(dimethylamino)methylene]-1H-1,2,3-triazolo[4,5-b]pyridinium 3-oxid hexafluorophosphate) (HATU).

For example, the intermediate comprising a therapeutic agent connected to a linker can be activated with TBTU, a peptide coupling reagent, prior to conjugation with the peptide compound or the antibody.

In one embodiment, the conjugate compound or antibody conjugate is purified following its synthesis.

Peptide compounds and antibodies disclosed herein are useful in the context of fusion proteins. For example, a fusion protein can be engineered by fusing a peptide compound or antibody herein disclosed, for example a peptide compound, to one or more proteins, or parts thereof such as functional domains. Fusion proteins can be engineered for example by recombinant DNA technology and expressed using a protein expression system such as a bacterial or mammalian protein expression system. In some embodiments, peptide linkers are added between proteins. In other embodiment, the fusion proteins do not comprise linkers connecting the proteins.

Commonly used protein expression systems include those derived from bacteria, yeast, baculovirus/insect, plants and mammalian cells and more recently filamentous fungi such as the Myceliophthora thermophile.

An aspect herein disclosed is a liposome, graphene, nanotube or nanoparticle comprising at least one peptide compound or antibody disclosed herein for use in inhibiting vasculogenic mimicry.

An aspect herein disclosed is a liposome, graphene, nanotube or nanoparticle comprising at least one peptide compound or antibody disclosed herein that targets Sortilin receptor.

An aspect herein disclosed is a liposome, graphene, nanotube or nanoparticle comprising at least one peptide compound or antibody disclosed herein for use in targeting Sortilin receptor.

Another aspect is a liposome, graphene, nanotube or nanoparticle coated with at least one compound, isolated antibody or antibody conjugate disclosed herein for use in inhibiting vasculogenic mimicry.

Another aspect is a liposome, graphene, nanotube or nanoparticle coated with at least one compound, isolated antibody or antibody conjugate disclosed herein that targets Sortilin receptor.

Another aspect is a liposome, graphene, nanotube or nanoparticle coated with at least one compound, isolated antibody or antibody conjugate disclosed herein for use in targeting Sortilin receptor.

Another aspect is a liposome, graphene, nanotube or nanoparticle loaded with at least one therapeutic agent, gene or siRNA; and the liposome or nanoparticle is coated with at least one compound, isolated antibody or antibody conjugate herein defined, for use in inhibiting vasculogenic mimicry. For example, the at least compound, conjugate compound, isolated antibody or antibody conjugate can be connected to the surface of the liposome or nanoparticle.

Different embodiments of liposomes, nanotubes, graphene or nanoparticles can be envisaged by the person skilled in the art. For example the liposome or nanoparticle can comprise at least one compound, isolated antibody or antibody conjugate herein disclosed coated on the surface of the liposome or nanoparticle and a therapeutic agent, for example an anticancer agent, within the liposome or nanoparticle. For example, the liposome or nanoparticle can comprise at least one compound herein disclosed coated on the surface of the liposome or nanoparticle and a therapeutic agent, for example an anticancer agent, within the liposome or nanoparticle. For example, the liposome or nanoparticle can comprise at least one peptide compound herein disclosed coated on the surface of the liposome or nanoparticle and a therapeutic agent, for example an anticancer agent, within the liposome or nanoparticle. For example, the liposome or nanoparticle can comprise at least one conjugate compound herein disclosed coated on the surface of the liposome or nanoparticle and a therapeutic agent, for example an anticancer agent, within the liposome or nanoparticle. For example, the liposome or nanoparticle can comprise at least one isolated antibody herein disclosed coated on the surface of the liposome or nanoparticle and a therapeutic agent, for example an anticancer agent, within the liposome or nanoparticle. For example, the liposome or nanoparticle can comprise at least one antibody conjugate herein disclosed coated on the surface of the liposome or nanoparticle and a therapeutic agent, for example an anticancer agent, within the liposome or nanoparticle. In addition, in some embodiments, the compound, peptide compound, conjugate compound, isolated antibody or antibody conjugate herein described can be associated, linked, or connected to one or more other compounds to form a multimer such as a dimer, a trimer or a tetramer, as well as branched peptides, optionally the peptide compound is cyclic. Such compound, isolated antibody or antibody conjugate can be connected together, for example via a covalent bond, an atom or a linker. For example, the multimer comprises more than one compound, isolated antibody or antibody conjugate. Methods for making multimeric (e.g. dimeric, trimeric) forms of peptide compounds or antibodies are described in U.S. Pat. No. 9,161,988 which is incorporated herein by reference in its entirety.

Other aspects of the present disclosure generally include methods of inhibiting vasculogenic mimicry comprising administering a therapeutically effective amount of at least one compound, isolated antibody or antibody conjugate herein disclosed to a subject in need thereof and/or contacting cells expressing Sortilin with at least one compound, isolated antibody and/or antibody conjugate herein disclosed. Other aspects include uses of the peptide compounds, conjugate compounds, antibodies and/or antibody conjugates described herein for inhibiting vasculogenic mimicry as well as in the manufacture of a medicament for inhibiting vasculogenic mimicry.

In an aspect, there is provided a method of inhibiting vasculogenic mimicry comprising administering to a subject in need thereof a therapeutically effective amount of at least one compound, isolated antibody and/or antibody conjugate as defined herein.

In another aspect, there is provided a method of inhibiting vasculogenic mimicry in cancerous tissues or cells expressing Sortilin, comprising contacting said cancerous tissues or cells with at least one compound, isolated antibody and/or antibody conjugate as defined herein.

In another aspect, there is provided a method of inhibiting vasculogenic mimicry in cancerous tissues or cells expressing Sortilin, comprising contacting said cancerous tissues or cells with at least one compound, isolated antibody and/or antibody conjugate as defined herein, wherein vasculogenic mimicry tube length or number of vasculogenic mimicry loops is decreased by at least about 20%, at least about 30%, at least about 40%, at least about 50%, at least about 60%, at least about 70%, at least about 80% or at least about 90% greater than untreated cancerous tissues or cells expressing Sortilin.

In another aspect, there is provided a method of inhibiting vasculogenic mimicry in cancerous tissues or cells expressing Sortilin, comprising contacting said cancerous tissues or cells with at least one compound, isolated antibody and/or antibody conjugate as defined herein, wherein vasculogenic mimicry tube length or vasculogenic mimicry loops is decreased by at least 1.2, at least 1.4, at least 1.6, at least 1.8, at least 2.0, at least 2.2 or at least 2.4 fold greater than cancerous tissues or cells expressing Sortilin treated with the at least one therapeutic agent.

In another aspect, there is provided a method of inhibiting vasculogenic mimicry in cancerous tissues or CD133 positive cells, comprising contacting said cancerous tissues or CD133 positive cells with at least one compound, isolated antibody and/or antibody conjugate as defined herein.

In an aspect, there is provided a method of treating cancer or aggressive cancer comprising administering to a subject in need thereof a therapeutically effective amount of at least one compound, isolated antibody and/or antibody conjugate as defined herein.

In another aspect, there is provided a method of treating cancer or aggressive cancer in a subject having cancerous tissues or cells expressing Sortilin, comprising contacting said cancerous tissues or cells with at least one compound, isolated antibody and/or antibody conjugate as defined herein.

In another aspect, there is provided a method of treating cancer or aggressive cancer in a subject having cancerous tissues or cells expressing Sortilin, comprising contacting said cancerous tissues or cells with at least one compound, isolated antibody and/or antibody conjugate as defined herein, wherein vasculogenic mimicry tube length or number of vasculogenic mimicry loops is decreased by at least about 20%, at least about 30%, at least about 40%, at least about 50%, at least about 60%, at least about 70%, at least about 80% or at least about 90% greater than untreated cancerous tissues or cells expressing Sortilin.

In another aspect, there is provided a method of treating cancer or aggressive cancer in a subject having cancerous tissues or cells expressing Sortilin, comprising contacting said cancerous tissues or cells with at least one compound, isolated antibody and/or antibody conjugate as defined herein, wherein vasculogenic mimicry tube length or vasculogenic mimicry loops is decreased by at least 1.2, at least 1.4, at least 1.6, at least 1.8, at least 2.0, at least 2.2 or at least 2.4 fold greater than cancerous tissues or cells expressing Sortilin treated with the at least one therapeutic agent.

In another aspect, there is provided a method of increasing stability and/or bioavailability of a therapeutic agent, comprising:
obtaining the conjugate compound or antibody conjugate disclosed herein, wherein said conjugate compound or antibody conjugate comprises said therapeutic agent, and
administering a therapeutically effective amount of said conjugate compound or antibody conjugate to a subject in need thereof.

In another aspect, there is provided a method of increasing stability and/or bioavailability of a therapeutic agent, comprising:
conjugating said therapeutic agent with the peptide compound or the antibody as defined herein to obtain a conjugate compound or antibody conjugate, and
administering a therapeutically effective amount of said conjugate compound or antibody conjugate to a subject in need thereof.

The conjugate compound or antibody conjugate herein disclosed may also provide greater tolerability compared to unconjugated therapeutic agents. For example, in the International application published as WO 2017/088058 and entitled PEPTIDE COMPOUNDS AND CONJUGATE COMPOUNDS FOR THE TREATMENT OF CANCER THROUGH RECEPTOR-MEDIATED CHEMOTHERAPY, filed Nov. 24, 2016 (herein incorporated by reference in its entirety), it has been shown in that Katana-drug conjugates are better tolerated compared to unconjugated therapeutic agents at an equivalent dose due to specific receptor targeting. In particular, in vivo studies showed that treatment with a conjugate compound had little effect on the body weight of tested mice thus demonstrating tolerability of the conjugate compound.

For example, there is provided herein a method of increasing tolerability of a therapeutic agent, comprising:
conjugating the therapeutic agent with a peptide compound or an isolated antibody herein disclosed to obtain a conjugate compound or an antibody conjugate, and
administering a therapeutically effective amount of the conjugate compound or the antibody conjugate to a subject in need thereof.

For example, there is provided herein a method of increasing tolerability of a therapeutic agent, comprising:
obtaining a conjugate compound or an antibody conjugate herein disclosed, wherein the conjugate compound or the antibody conjugate comprises the therapeutic agent, and
administering a therapeutically effective amount of the conjugate compound or the antibody conjugate to a subject in need thereof.

For example, there is provided a use of a peptide compound, a conjugate compound, an antibody and/or an antibody conjugate herein disclosed, for increasing tolerability of a therapeutic agent.

In another aspect, there is provided a use of at least one compound, isolated antibody and/or antibody conjugate as defined herein for inhibiting vasculogenic mimicry.

In another aspect, there is provided a use of at least one compound, isolated antibody and/or antibody conjugate as defined herein for targeting Sortilin receptor.

In another aspect, there is provided a use of at least one compound, isolated antibody and/or antibody conjugate as defined herein for inhibiting vasculogenic mimicry involving sortilin expression.

In another aspect, there is provided a use of at least one compound, isolated antibody and/or antibody conjugate as defined herein for inhibiting vasculogenic mimicry in cancerous tissues or cells expressing Sortilin.

In another aspect, there is provided a use of at least one compound, isolated antibody and/or antibody conjugate as defined herein for treatment of cancer or aggressive cancer.

In another aspect, there is provided a use of at least one compound, isolated antibody and/or antibody conjugate as defined herein for treatment of cancer or aggressive cancer involving sortilin expression.

In another aspect, there is provided a use of at least one compound, isolated antibody and/or antibody conjugate as defined herein for treatment of cancer or aggressive cancer in cancerous tissues or cells expressing Sortilin.

In another aspect, there is provided a use of at least one compound, isolated antibody or antibody conjugate for decreasing vasculogenic mimicry tube length in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In another aspect, there is provided a use of at least one compound, isolated antibody or antibody conjugate for decreasing number of vasculogenic mimicry loops in cancerous tissues or cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cancerous tissues or cells expressing Sortilin.

In another aspect, there is provided a use of at least one compound, isolated antibody or antibody conjugate as defined herein for decreasing vasculogenic mimicry tube length in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In another aspect, there is provided a use of at least one compound, isolated antibody or antibody conjugate as defined herein for decreasing number of vasculogenic mimicry loops in cancerous tissues or cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cancerous tissues or cells expressing Sortilin treated with the at least one therapeutic agent.

In another aspect, there is provided a use of at least one compound, isolated antibody or antibody conjugate as defined herein for inhibiting vasculogenic mimicry in cancerous tissues or cells expressing Sortilin, comprising contacting said cancerous tissues or cells with at least one compound, isolated antibody and/or antibody conjugate as defined herein, wherein vasculogenic mimicry tube length or number of vasculogenic mimicry loops is decreased by at least about 20%, at least about 30%, at least about 40%, at least about 50%, at least about 60%, at least about 70%, at least about 80% or at least about 90% greater than untreated cancerous tissues or cells expressing Sortilin.

In another aspect, there is provided a use of at least one compound, isolated antibody or antibody conjugate as defined herein for inhibiting vasculogenic mimicry in cancerous tissues or cells expressing Sortilin, comprising contacting said cancerous tissues or cells with at least one compound, isolated antibody and/or antibody conjugate as defined herein, wherein vasculogenic mimicry tube length or vasculogenic mimicry loops is decreased by at least 1.2, at least 1.4, at least 1.6, at least 1.8, at least 2.0, at least 2.2 or at least 2.4 fold greater than cancerous tissues or cells expressing Sortilin treated with the at least one therapeutic agent.

In another aspect, there is provided a use of at least one compound, isolated antibody or antibody conjugate as defined herein for decreasing vasculogenic mimicry tube length or number of vasculogenic mimicry loops in cancerous tissues or cells expressing Sortilin by at least about 20%, at least about 30%, at least about 40%, at least about 50%, at least about 60%, at least about 70%, at least about 80% or at least about 90% greater than untreated cancerous tissues or cells expressing Sortilin.

In another aspect, there is provided a use of at least one compound, isolated antibody or antibody conjugate as defined herein for decreasing vasculogenic mimicry tube length or number of vasculogenic mimicry loops in cancerous tissues or cells expressing Sortilin by at least 1.2, at least 1.4, at least 1.6, at least 1.8, at least 2.0, at least 2.2 or at least 2.4 fold greater than cancerous tissues or cells expressing Sortilin treated with the at least one therapeutic agent.

In another aspect, there is provided a use of at least one compound, isolated antibody and/or antibody conjugate as defined herein for treatment of cancer or aggressive cancer in CD133 positive cells.

In another aspect, there is provided a use of a conjugate compound or an antibody conjugate as defined herein for increasing stability and/or bioavailability of at least one therapeutic agent.

In another aspect, there is provided a use of one compound, isolated antibody and/or antibody conjugate as defined herein in the manufacture of a medicament for inhibiting vasculogenic mimicry.

In another aspect, there is provided a use of one compound, isolated antibody and/or antibody conjugate as defined herein in the manufacture of a medicament for targeting vasculogenic mimicry.

In another aspect, there is provided a use of one compound, isolated antibody and/or antibody conjugate as defined herein in the manufacture of a medicament for inhibiting vasculogenic mimicry involving sortilin expression.

In another aspect, there is provided a use of one compound, isolated antibody and/or antibody conjugate as defined herein in the manufacture of a medicament for inhibiting vasculogenic mimicry in cancerous tissues or cells expressing Sortilin.

In another aspect, there is provided a use of one compound, isolated antibody and/or antibody conjugate as defined herein in the manufacture of a medicament for inhibiting vasculogenic mimicry in CD133 positive cells.

In another aspect, there is provided a use of one compound, isolated antibody and/or antibody conjugate as defined herein in the manufacture of a medicament for treatment of cancer or aggressive cancer.

In another aspect, there is provided a use of one compound, isolated antibody and/or antibody conjugate as defined herein in the manufacture of a medicament for treatment of cancer or aggressive cancer involving Sortilin expression.

In another aspect, there is provided a use of one compound, isolated antibody and/or antibody conjugate as defined herein in the manufacture of a medicament for treatment of cancer or aggressive cancer in cancerous tissues or cells expressing Sortilin.

In another aspect, there is provided a use of one compound, isolated antibody and/or antibody conjugate as defined herein in the manufacture of a medicament for treatment of cancer or aggressive cancer in CD133 positive cells.

For example, the at least one therapeutic compound comprised in the conjugate compound or the antibody conjugate and/or used in the manufacture of a medicament to inhibit vasculogenic mimicry is an anticancer agent. For example, the anticancer agent is chosen from docetaxel, cabazitaxel, aldoxorubicin maytansinoids, auristatins, calicheamicins, amatoxins, amanitin, and doxorubicin.

For example, the at least one therapeutic compound comprised in the conjugate compound or the antibody conjugate and/or used in the manufacture of a medicament to inhibit vasculogenic mimicry is a phytochemical. For example, the phytochemical is curcumin.

For example, the phytochemical is chosen from curcumin, omega-3, white willow bark, green tea, catechins, pycnogenol, Boswellia serrata resin, resveratrol, uncaria tomentosa, capsaicin, anthocyanins/anthocyanidins, flavanoids, olive oil compounds, chlorogenic acid and sulfopharaphane.

In another aspect, there is provided a use at least one compound herein disclosed, at least one isolated antibody herein disclosed, or at least one conjugated antibody herein disclosed, for inhibiting vasculogenic mimicry, in combination with a therapeutic agent such as cytotoxics, toxins and anticancer peptides; an immunodulator such anti-PD1 and anti-PDL1; an anticancer delivery system, an antiangiogenic agent; and/or radiotherapy.

In a further aspect, there is provided a use at least one compound herein disclosed, at least one isolated antibody herein disclosed, or at least one conjugated antibody herein disclosed, for targeting Sortilin receptor, in combination with a therapeutic agent such as cytotoxics, toxins and anticancer peptides; an immunodulator such anti-PD1 and anti-PDL1; an anticancer delivery system, an antiangiogenic agent; and/or radiotherapy.

Further embodiments of the present disclosure will now be described with reference to the following Examples. It should be appreciated that these Examples are for the purposes of illustrating embodiments of the present disclosure, and do not limit the scope of the disclosure.

### EXAMPLES

### Example 1: Compositions

### Peptides Targeting Sortilin

The first family of Katana peptides that target Sortilin is derived from a bacterial cell penetrant protein, whereas the second family is rather based on an optimized primary sequence derived from the Sortilin ligands, progranulin and neurotensin (Table 1). These peptides have been described in PCT/CA2016/051379: PEPTIDE COMPOUNDS AND CONJUGATE COMPOUNDS FOR THE TREATMENT OF CANCER THROUGH RECEPTOR-MEDIATED CHEMOTHERAPY and in U.S. patent application Ser. No. 62/510,381): CONJUGATES AND USES THEREOF FOR TREATING INFLAMMATORY DISEASES, herein incorporated by reference.

TABLE 1
Amino acid sequences of Katana peptide families.

| | |
|---|---|
| Amino acid sequence | Amino acid length |

Katana Biopharma Peptide (KBP) Family 1:

| | |
|---|---|
| KBP-101: IKLSGGVQAKAGVINMDKSESM | 22 |
| KBP-102: Succinyl-IKLSGGVQAKAGVINMFKSESY | 22 |
| KBP-103: IKLSGGVQAKAGVINM FKSESYK(Biotin) | 23 |
| KBP-104: GVQAKAGVINMDKSESMY | 17 |
| KBP-105: Acetyl-GVRAKAGVRNMFKSESY | 17 |
| KBP-106: Acetyl-GVRAKAGVRN(Nle)FKSESY | 17 |
| KBP-106-Cys: Acetyl-GVRAKAGVRN(Nle)FKSESY | 18 |

Katana Biopharma Peptide (KBP) Family 2:

| | |
|---|---|
| KBP-201: YKSLRRKAPRWDAPLRDPALRQLL | 24 |
| KBP-202: YKSLRRKAPRWDAYLRDPALRQLL | 24 |
| KBP-203: YKSLRRKAPRWDAYLRDPALRPLL | 24 |

### Methods

### Generation of Katana-Peptide Drug Conjugate(s)

Docetaxel and Doxorubicin were first chosen for the proof of principle for cytotoxic drugs, whereas Curcumin was selected among phytochemicals. Docetaxel is a semi-synthetic analogue of Paclitaxel, an extract from the bark of the rare Pacific yew tree Taxus brevifolia. This drug has been approved by the FDA (National Cancer Institute) for the treatment of locally advanced or metastatic cancers including breast cancer, ovarian cancer, head and neck cancer, gastric cancer, hormone-refractory prostate cancer and non small-cell lung cancer. Docetaxel can be used as a single agent or in combination with other chemotherapeutic drugs depending of specific cancer type and stage. Doxorubicin is an anthracycline antitumor antibiotic (note: in this context, this does not mean it is used to treat bacterial infections) closely related to the natural product Daunomycin and, like all anthracyclines, works by intercalating DNA, with the most serious adverse effect being life-threatening heart damage (National Cancer Institute). It is approved to be used alone or with other drugs to treat: acute lymphoblastic leukemia (ALL), acute myeloid leukemia (AML), breast cancer, gastric (stomach) cancer, Hodgkin lymphoma, neuroblastoma, non-Hodgkin lymphoma, ovarian cancer, small cell lung cancer, soft tissue and bone sarcomas, thyroid cancer, transitional cell bladder cancer and Wilms tumor. Curcumin (diferuloylmethane) is a yellow pigment present in the spice turmeric (Curcuma longa) that has been associated with antioxidant, anti-inflammatory, anticancer, antiviral and antibacterial activities (23).

Cytotoxic drugs (Docetaxel, Cabazitaxel, Doxorubicin) or phytochemicals (curcumin) can be conjugated on the peptide using amine conjugation strategies. Briefly, Docetaxel can be conjugated to Katana peptide(s) on free amines available on the peptide (lysine or amino-terminal) by forming a peptide bond (amide bond) with activated-Docetaxel. Various conjugation strategies for the peptides have previously been described in PCT/CA2016/051379 and U.S. 62/510,381, herein incorporated by reference.

### Antibody Labelling

Labeling of anti-Sortilin antibody was performed with the Alexa Fluor 488 protein labeling kit from Invitrogen according to manufacturer's instructions.

### Cell Surface Binding Assay

Human ES-2 ovarian cancer cells were incubated at 4° C. with anti-Sortilin-Alexa488 antibody (1 μg/ml) for 30 minutes followed or not by trypsinization to assess the binding antibodies at the cell surface.

### Vasculogenic Mimicry

Malignant solid tumours need blood supply to promote growth and metastasis. In the past, angiogenesis, one type of neovascularization in embryo development, was regarded as the only form of supporting tumour blood supply (24). Anti-angiogenic therapies targeting endothelial cells, as a potential and promising treatment target, have received much attention and investigation. Many anti-angiogenic drugs have been used to prevent tumour growth and metastasis. However, the drugs' effects on cancer progression was restricted and unsatisfactory, which indicated that there may be other blood supply forms in tumour tissues besides angiogenesis. In 1999, it was observed that a highly patterned vessel-like channel structure in highly aggressive and metastatic human melanoma in which red blood cells were detected (28). Endothelial cells were not detected in these channels by light microscopy, transmission electron microscopy or immunohistochemical detection of endothelial cell markers (such as CD34, CD31). Subsequently, they reported that this structure, different from angiogenesis, was comprised of a basement membrane and mainly lined by tumour cells. Other studies also confirmed that vasculogenic mimicry provides an important perfusion pathway for malignant tumours through sufficient blood and nutrition supply (24-27). It is now recognized that vasculogenic mimicry plays a significant role in tumour growth.

Vasculogenic mimicry is linked to tumour malignancies, including invasion and metastasis (24-27). There exists a junction in tumour tissues between tumour-lined vascular channels and endothelial-lined blood vessels. Through this structure, tumour cells lining the inner network channel surface are directly exposed to the blood, significantly increasing transfer opportunities. Vasculogenic mimicry describes a process by which cancer cells establish an alternative perfusion pathway in an endothelial cell-free manner. Vasculogenic tumour cells adopt a way of embryonic vasculogenesis and directly form primitive, immature blood vessels consisting of various capillary-like structures, tubes and networks. Vasculogenic mimicry has been reported in various malignant tumours and is known to play a role in cancer progression and metastasis. Vasculogenic mimicry may exist in various malignant tumours (24, 28, 29), including melanoma, ovarian cancer, breast cancer, prostate cancer, osteosarcoma, bladder cancer, colorectal cancer, hepatocellular cancer, gastric cancer and lung cancer. In breast cancer, vasculogenic mimicry has been reported to be highest in triple negative breast cancer (TNBC) specimens (30). In this latter study, CD133+ cells with cancer stem cell characteristics were associated with vasculogenic mimicry in TNBC. A meta-analysis to evaluate the influence of vasculogenic mimicry on the 5-year survival of 3062 cancer cases involved in 15 types of malignant tumours showed that vasculogenic mimicry is associated with more aggressive tumor phenotype and a poor 5-year overall survival of cancer patients (24). Without wishing to be bound theory, vasculogenic mimicry provides growing tumors with a new mechanism of blood perfusion and a potential dissemination route. Further, Sortilin receptor may be involved in cancer cell proliferation, cancer growth, as well as cancer cell migration and invasion.

In this disclosure, the potential role of Sortilin in vasculogenic mimicry was investigated. As disclosed herein, Sortilin is essential in the formation of vasculogenic mimicry tubular structures. More importantly, conjugates between anticancer drugs (Doxorubicin, Cabazitaxel, Aldoxorubicin, Docetaxel) or phytochemicals (Curcumin) and peptide(s) that target Sortilin strongly inhibit the formation of vasculogenic mimicry. Furthermore, results described herein show that the formation of 3D-tubular structures associated to vasculogenic mimicry is inhibited by a mAb directed against Sortilin.

### Results

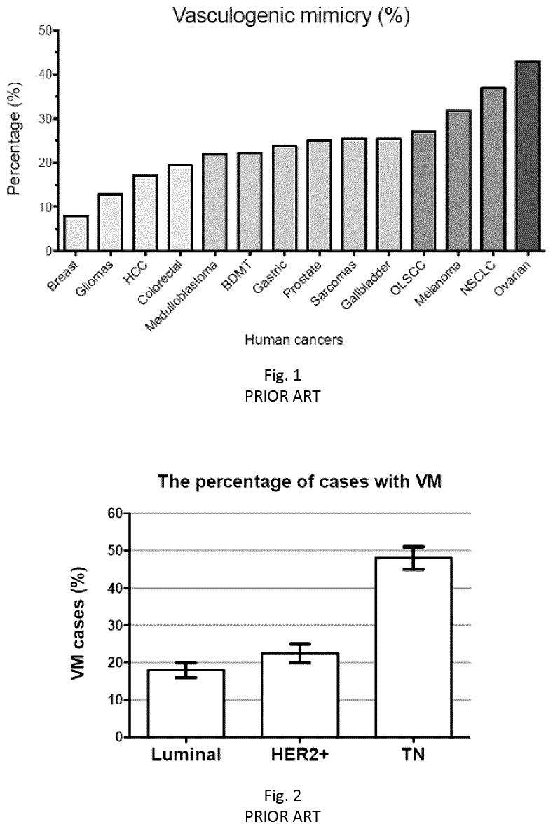

FIG. 1 and FIG. 2 show the extent of vasculogenic mimicry in different human cancers and breast cancers, respectively (24, 30). Ovarian cancer and triple negative (TN) breast cancer present the highest levels of vasculogenic mimicry.

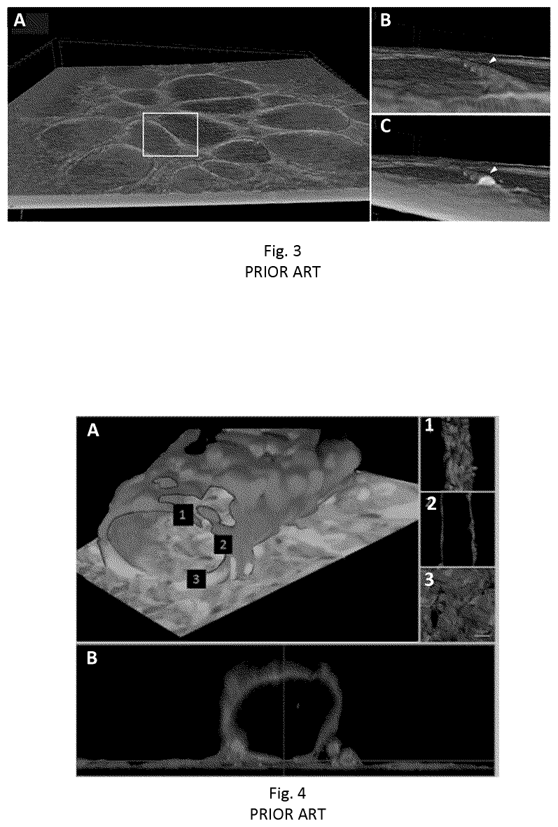

FIG. 3 and FIG. 4 describe the formation of vasculogenic mimicry observed in vitro for ovarian cancer cells (31). 3D-reconstruction of X-ray microtomography of the landscape of a 4-days 3D-culture of ovarian cancer cells on Matrigel (FIG. 3A) is showing a reconstructed view of elevated structures with tubular-like appearances. In addition, the structures within the white rectangle are shown in higher magnification in FIG. 3B and FIG. 3C, with the arrowhead denoting a seemingly tubular structure projecting above the flat cell aggregates. A cross-section of this structure demonstrates an air-filled space with an estimated diameter of 50 μm (FIG. 3C). FIG. 4 shows confocal microscopy of vasculogenic mimicry tubular structure using SKOV3 expressing the green fluorescent protein (GFP). Overall, Z-stack reconstruction images in FIG. 4A demonstrates the presence of a cell containing tubular structure with a continuous upper monolayer [1], a central walled structures, a hollow center [2] and a continuous lower monolayer [3]. Computer-generated cross-section in FIG. 4B clearly shows lumen-containing tubular structures.

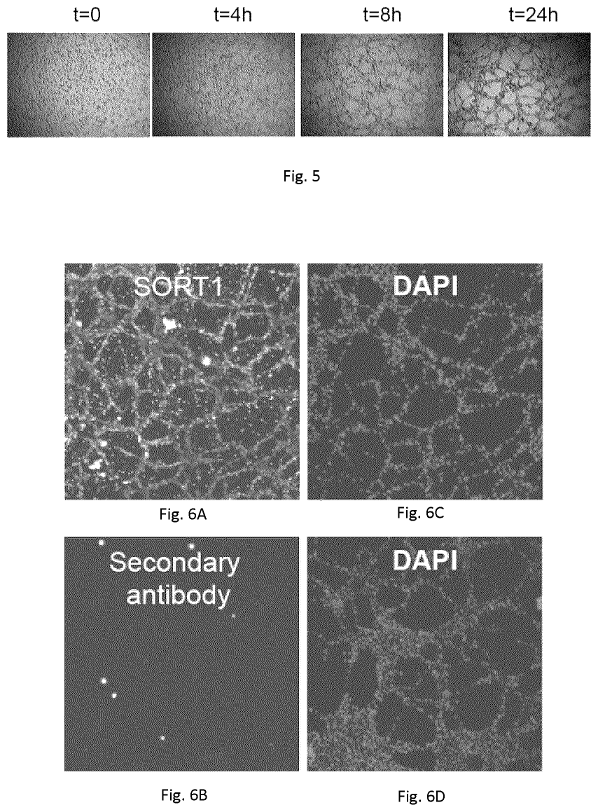

FIG. 5 shows microscopy images demonstrating that ES-2 ovarian cancer cells are forming vasculogenic mimicry. 3D-tubular structures in ES-2 cells were rapidly formed and observed within 4 h after seeding on Matrigel.

FIG. 6 shows the detection of Sortilin in 3D-tubular structures of ES2 ovarian cancer cells. ES-2 ovarian cancer cells were seeded on Matrigel. FIG. 6A shows that after 12 hrs, Sortilin (SORT1) was detected in 3D-tubular structures by confocal microscopy using a rabbit anti-Sortilin antibody. FIG. 6B shows a control experiment carried out with only the secondary anti-rabbit antibody. Overall results indicate that Sortilin detection is specific. FIG. 6C and FIG. 6D show DAPI staining of ES-2 cancer cell nuclei, which serves as a control, to demonstrate the presence of the 3D-tubular structures under conditions used for anti-Sortilin (FIG. 6A) and secondary antibody (FIG. 6B) detections.

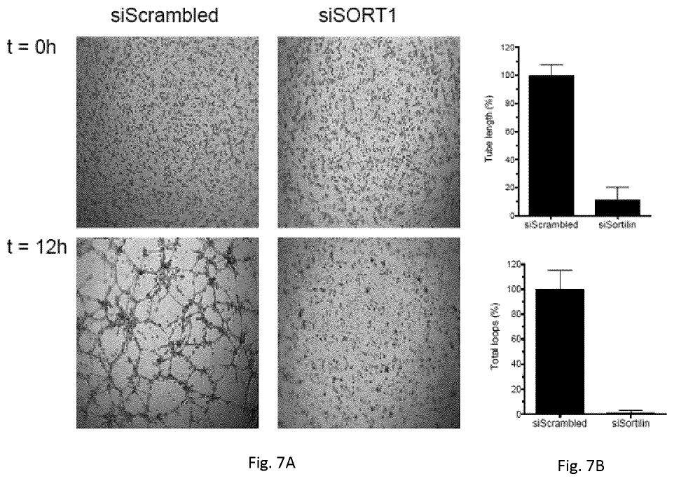

FIG. 7 shows the effect of Sortilin gene silencing on the formation of vasculogenic mimicry by ES-2 ovarian cancer cells. When Sortilin expression was specifically repressed, the 3D-tubular structures observed during vasculogenic mimicry formation were strongly inhibited when compared to scrambled siRNA (FIG. 7A). Images were sent to Onimagin Technologies for quantitative analysis of total loops and total tube length using Wimasis Image Analysis software (FIG. 7B). Results indicate that both the number of loops and the total tube length are inhibited by more than 90% following Sortilin gene silencing with siRNA. These results clearly demonstrate that Sortilin is a key player in the formation of 3D-tubular structures in vasculogenic mimicry.

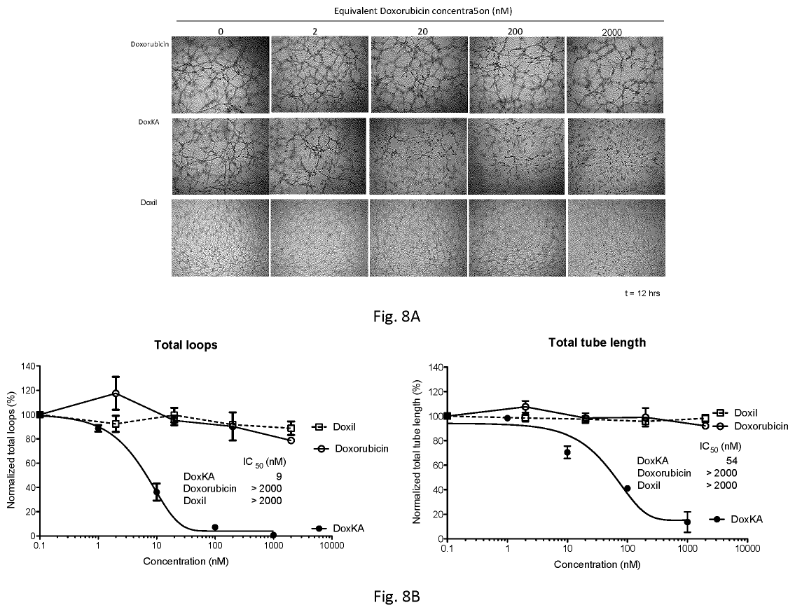

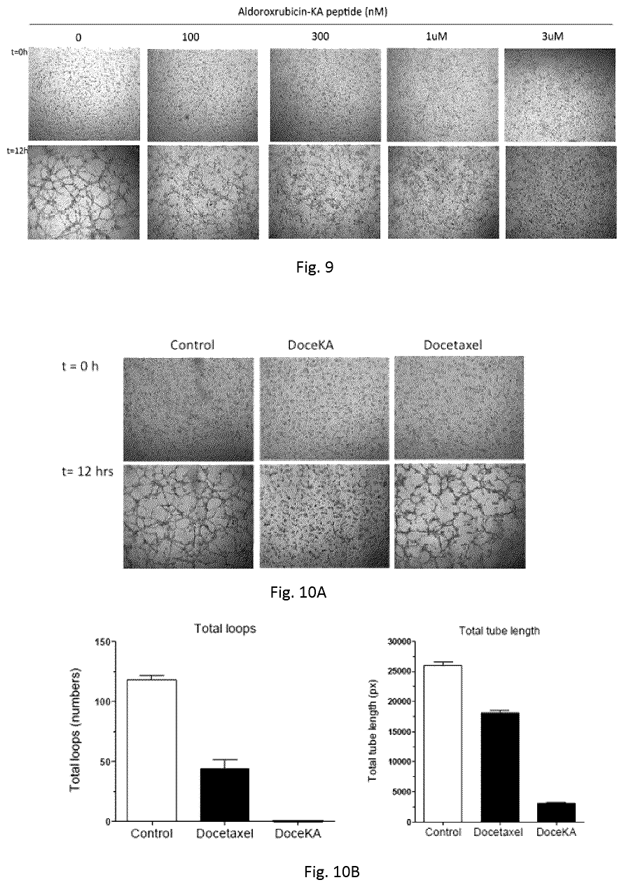

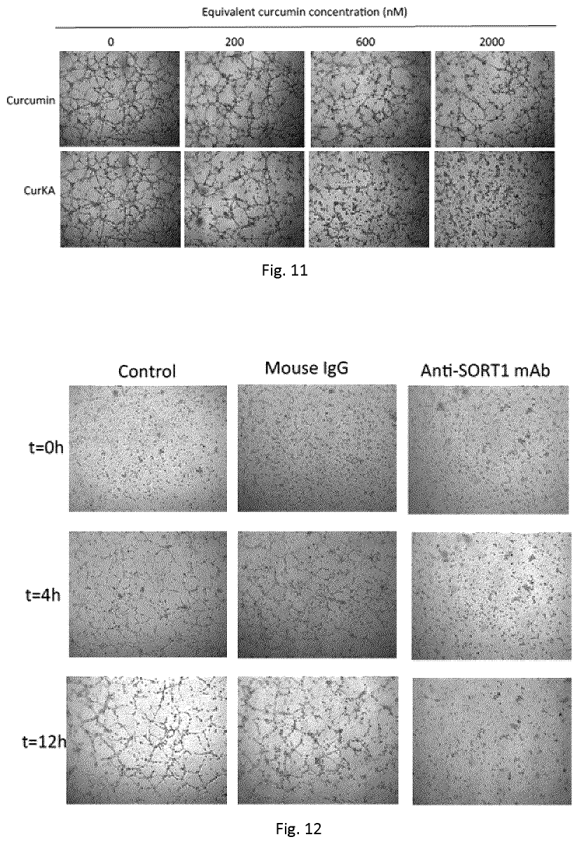

FIGS. 8, 9, 10 and 11 show examples of vasculogenic mimicry inhibition by novel conjugates between anticancer drugs (Doxorubicin, Cabazitaxel, Aldoxorubicin, Docetaxel) or phytochemicals (Curcumin) and peptide(s) that target Sortilin. In FIG. 8, vasculogenic mimicry of ES-2 ovarian cancers on Matrigel was performed in the presence of unconjugated Doxorubicin or Doxorubicin conjugate compound (DoxKA). FIG. 8A shows that after 24 hrs, increasing concentrations of DoxKA strongly inhibited the formation of vasculogenic mimicry. FIG. 8B shows 3D-tubular structures analyzed with Wimasis Images Analysis software. Results were plotted as a function of drug concentration. DoxKA IC50 values obtained for the total tube length and the number of loops are in the low nM range (5-10 nM) as indicated in FIG. 8B. A second example with a Doxorubicin derivative (Aldoxorubicin)-drug conjugate compound is presented in FIG. 9. Conjugation of Aldoxorubicin to the peptide is different than with Doxorubicin. In the case of Aldoxorubicin, the drug was conjugated on a Cysteine amino acid residue that was added to the C-terminus of the peptide. The linker used is sensitive to acidic pH rather than esterases as in the case of Doxorubicin. Ratio of incorporation is also different for AldoxKA since only one molecule of Aldoxorubicin was conjugated on the peptide compared to 2 molecules of Doxorubicin in the DoxKA conjugate. This demonstrates the flexibility of the platform starting with two different Doxorubicin molecules. A third example with an anticancer-drug conjugate compound is presented in FIG. 10A and FIG. 10B. Docetaxel-conjugate compound (DoceKA) caused a stronger inhibition of vasculogenic mimicry formation compared to Docetaxel. IC50 values for the number of loops is about 30 μM for this conjugate. Another example is shown in FIG. 11 with a phytochemical-conjugate compound. For this class of molecules, Curcumin was conjugated to Sortilin targeted peptide (CurKA). Curcumin has been suggested to inhibit tube formation of SK-Hep-1 hepatocellular carcinoma cells (32). Within the range of 3-30 μM, Curcumin inhibited tube formation of the cells in a dose-dependent manner, with 18-92% inhibition being observed. FIG. 11 clearly show that CurKA conjugate affects the formation of ES-2 ovarian vasculogenic mimicry in the nM concentration. CurKA has a stronger effect on the 3D-tube formation of ES-2 ovarian cancer cells when compared to unconjugated Curcumin.

FIG. 12 demonstrates that anti-Sortilin mAb inhibits vasculogenic mimicry. ES-2 ovarian cancer cells were seeded on Matrigel in the presence of either a non-specific mouse IgG or an anti-Sortilin mAb at a concentration of 12 nM. Images taken 12 hrs later show that the anti-Sortilin mAb prevented the formation of 3D-tubular structures of ES-2 ovarian cancer cells whereas the mouse IgG had no effect when compared to the control. This vasculogenic mimicry inhibition by the anti-Sortilin mAb is very similar to the one observed when Sortilin gene silencing with siRNA was performed (FIG. 6).

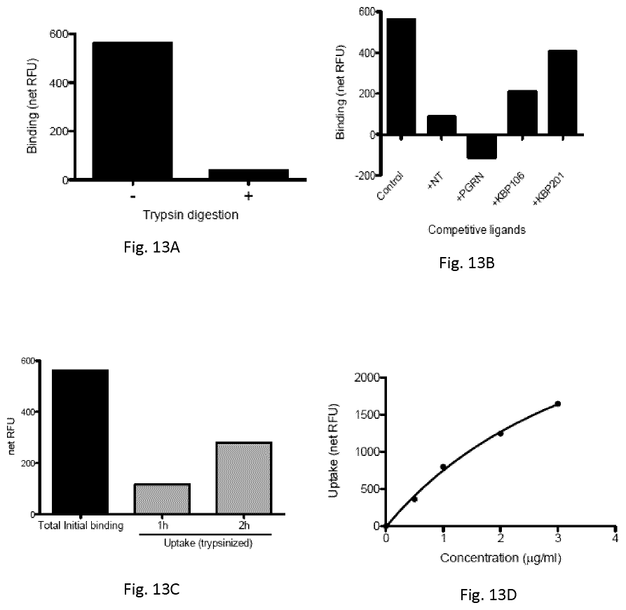

FIG. 13A shows the binding and internalization of Anti-SORT1 mAb. FIG. 13A demonstrates that an anti-Sortilin antibody labeled with the fluorescent dye Alexa488 binds to the receptor Sortilin at the cell surface of ovarian cancer cells. Anti-Sortilin antibody was labelled with Alexa Fluor 488 to generate anti-Sortilin-Alexa488 antibody. Human ES-2 ovarian cancer cells were incubated with anti-Sortilin-Alexa488 antibody (1 μg/ml) for 30 minutes followed or not by trypsinization to assess the binding at the cell surface. Results clearly demonstrate that the major portion of the fluorescence signal was caused by the binding to the Sortilin receptor at the cell surface since trypsinization reduced the fluorescence levels by more than 90%. In FIG. 13B, the binding of the anti-Sortilin-Alexa488 antibody (1 μg/ml, 30 minutes) at the cell surface of the human ES-ovarian cancer cells was inhibited by Sortilin ligands (neurotensin (NT) and progranulin (PGRN)). In addition, the incubation in the presence of Katana peptides (KBP106 and KBP201), which are also recognized by Sortilin, strongly reduced the binding of the fluorescent antibody. Overall, these results indicate that the interactions of Sortilin ligands and Katana peptides prevent the labeling and recognition of the receptor by the fluorescent anti-Sortilin antibody.

Results in FIG. 13C show that the anti-Sortilin antibody can internalize a fluorescent dye conjugate into cancer cells. In this experiment, binding of the anti-Sortilin-Alexa488 antibody (1 μg/ml, 30 minutes) on human ES-2 ovarian cancer cells was first performed at 4° C., then the cells were washed to remove unbound fluorescent antibody and cells were incubated at 37° C. for 1 or 2 hrs and then trypsinized. This allows the internalization of the anti-Sortilin-Alexa488 conjugate into the cells over time. Fluorescence associated with internalized fluorescent antibody was then quantified by flow cytometry and results indicate that about 50% of anti-Sortilin-Alexa488 antibody, which was first bound on the cell surface, was internalized within 2 hrs. Internalization of anti-Sortilin-Alexa488 antibody was measured with increasing concentrations (FIG. 13D). Results demonstrate that the internalization of the anti-Sortilin-Alexa488 fluorescent conjugate increased as a function of fluorescent conjugate concentration and was saturable.

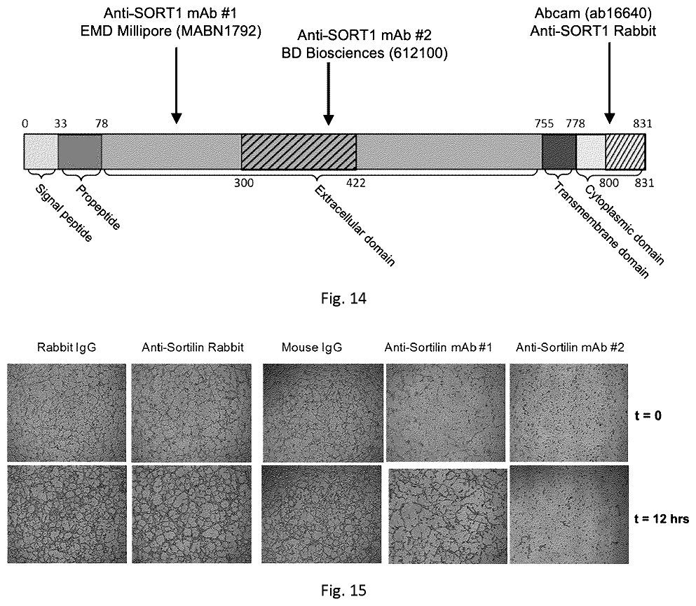

FIG. 14 shows a schematic diagram of different regions of Sortilin and the regions where the anti-Sortilin antibodies used in this disclosure were generated against. Anti-Sortilin mAb #1 (Anti-SORT1 mAb #1) was obtained from EMB Millipore (Cat #MABN1792). The immunogen of this antibody is in the extracellular domain of Sortilin, i.e. amino acid residues 78-755. Anti-Sortilin mAb #2 (Anti-SORT1 mAb #2) was obtained from BD Bioscience (Cat #612100). The immunogen of this antibody is amino acid residues 300-422 of Sortilin corresponding to part of the extracellular domain of this protein.

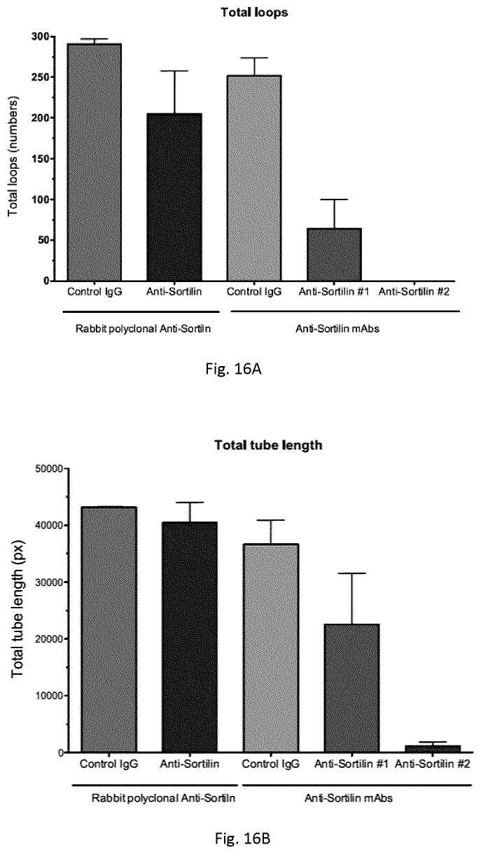

FIG. 15 shows inhibition of vasculogenic mimicry of ovarian cancer cells by anti-Sortilin antibodies. ES-2 ovarian cancer cells were seeded on Matrigel in the presence of the vehicle (Control), mouse IgG (4 μg/mL (about 25 nM)) or anti-SORT1 mAb #1 (4 μg/mL (about 25 nM)), or anti-SORT1 mAb #2 (4 ug/mL (about 25 nM)). After 12 hours of exposing ES-2 cells to the antibodies, the formation of 3D-tubular structure was strongly inhibited by Anti-Sortilin mAb #1 and Anti-Sortilin mAb #2, as seen by the impact of these mAbs to total loops (FIG. 16A) and total tube length (FIG. 16B).

### Discussion

Without wishing to be bound by theory, it is believed that vasculogenic mimicry is a phenomenon associated with more aggressive tumor phenotype and poor prognosis in patients with various cancers. Because the overexpression of Sortilin in many types of cancers have been associated with their survival, invasion and progression, targeting this receptor with conjugate compound, antibody or conjugate antibody may provide an efficient way to increase the binding/internalization of cancer drugs within these tumors. Results disclosed herein clearly indicate that conjugate compound, mAb and their derivatives targeting/interacting with Sortilin led to a strong inhibition of vasculogenic mimicry.

### Example 2: Sortilin Receptor-Mediated Cancer Therapy: A Targeted Approach for Vasculogenic Mimicry Inhibition in Ovarian and Breast Cancers

### Background

Vasculogenic mimicry is defined as the formation of microvascular channels by aggressive, metastatic and genetically deregulated tumor cells. This microcirculation system, independent of endothelial cells, provides oxygen and nutrients to tumor cells. Vasculogenic mimicry has been occurring in ovarian cancer and in triple negative breast cancer (TNBC) and shown to correlate with decreased overall cancer patient survival. Such process contributes, in part, to current chemoresistance and facilitates tumor progression as well as dissemination of cancer metastases. Therefore targeting vasculogenic mimicry in ovarian and TNBC tumors may contribute to cancer treatment. Sortilin, a scavenging receptor, is already known to play a prime function in cancer cells; however we are reporting herein that it further plays a new role in vasculogenic mimicry. Targeting vasculogenic mimicry with peptide-drug conjugates through Sortilin was explored in ovarian and TNBC.

### Methods

In vitro vasculogenic mimicry of cancer cells was assessed using a Matrigel tube formation assay. In brief, each well of a 96-well plate was pre-coated with Matrigel. ES-2 ovarian cancer or MDA-MB-231 TNBC cell suspensions were seeded on top of Matrigel. 3D-tubular structures were analyzed and quantified using Wimasis Analysis software. Real-time cell migration was evaluated using the ×CELLigence system. Sortilin gene silencing was performed with specific siRNA. Effects of Doxorubicin-KA-peptide conjugate (DoxKA) KBA-106 represented by formula Acetyl-GVRAK(doxorubicin)AGVRN(Nle)FK(doxorubicin)SESY (Formula (XXVIII)) and Docetaxel-KA-peptide conjugate (DoceKA) represented by formula Acetyl-GVRAK(docetaxel)AGVRN(Nle)FK(docetaxel)SESY (Formula (XXIII)) targeting Sortilin on vasculogenic mimicry and cell migration were determined.

### Results

It was found that Sortilin is expressed in 3D-tubular structures on Matrigel and essential for vasculogenic mimicry in ovarian cancer and TNBC. When Sortilin expression was specifically repressed, the 3D-tubular structures observed during vasculogenic mimicry were strongly inhibited. Furthermore, anti-Sortilin mAbs prevented vasculogenic mimicry to occur, whereas the non-specific IgG had no effect. Interestingly, the peptide-drug conjugates, DoxKA or DoceKA that target Sortilin, caused a stronger inhibition of vasculogenic mimicry than unconjugated free drugs. DoxKA and DoceKA IC₅₀ values for vasculogenic mimicry inhibition ranged within low nM to pM concentrations. Both DoxKA and DoceKA conjugates also inhibited ES-2 and MDA-MB231 cancer cells migration in a Sortilin-dependent process.

### Conclusions

Our results identify Sortilin receptor as a key player in vasculogenic mimicry. More importantly, the design of peptide-drug conjugates targeting Sortilin is proved to strongly inhibit vasculogenic mimicry, a phenomenon associated with a more aggressive tumor phenotype and poor prognosis in patients with TNBC and ovarian cancer. Our peptide-drug conjugation platform provides the pre-clinical molecular insight to a receptor-mediated chemotherapy and to a more efficient therapeutic management of Sortilin-positive cancers.

### Example 3: Increasing Potency and Safety of Anticancer Drugs Through Sortilin Receptor-Mediated Cancer Therapy: A Targeted Approach for the Treatment of Ovarian Cancer

### Background

The development of personalized therapies against ovarian cancer remains highly challenging in current modem oncology. One strategy to achieve greater selectivity and better anticancer drug delivery into cancer cells is to conjugate cytotoxic agents to specific peptide ligands that selectively target receptors abundantly and/or exclusively expressed on these cells. Increased expression of Sortilin, a scavenging receptor, has been clinically observed in invasive ovarian cancer biopsies, and correlated with tumor grades. In light of this, we developed a peptide conjugation platform and a Sortilin receptor-mediated vectorization strategy to increase cell targeting selectivity and cell delivery efficacy of anticancer agents.

### Methods

As a proof-of-concept, Doxorubicin was conjugated to a Sortilin binding peptide (KA-peptide). In vitro, intracellular delivery of the Doxorubicin-KA-peptide conjugate (DoxKA) KBB-106 represented by formula Acetyl-GVRAK(doxorubicin)AGVRN(Nle)FK(doxorubicin)SESY (Formula (XXVIII)) was assessed in ES-2 and SKOV-3 ovarian cancer cell line models using flow cytometry and fluorescent microscopy. Sortilin gene silencing was performed with specific siRNA. DoxKA efficacy and safety were evaluated in vivo using ES-2 (CD1 nude mice) and SKOV-3 (athymic mice) subcutaneous xenograft models.

### Results

Uptake of DoxKA was observed in both Sortilin-positive ovarian cancer cell lines tested and was reduced when Sortilin expression was specifically silenced or upon competition with the Sortilin ligands Neurotensin and Progranulin. Results indicate that the uptake of DoxKA occurs via Sortilin-mediated endocytosis in contrast to simple diffusion for Doxorubicin. DoxKA was found to bypass the P-glycoprotein (P-gp) efflux pump in MDCK-MDR1 cells overexpressing P-gp as the uptake of DoxKA was unaffected by the P-gp inhibitor Cyclosporin A. In vivo, DoxKA showed lower potential side effects than Doxorubicin alone did, with decreased accumulation in healthy tissues such as heart and ovary. DoxKA caused a more potent inhibition of human ovarian tumor xenografts growth in mice and was better tolerated (absence of leukopenia and neutropenia) than the unconjugated Doxorubicin at an equivalent dose.

### Conclusions

These results support the future clinical use of this platform to generate novel personalized therapeutics with specific targeting of Sortilin-positive tumors in the next stage of development in phase 1 clinical trial.

### Example 4: Docetaxel-Peptide Conjugate for the Treatment of Sortilin-Positive Triple-Negative Breast Cancer

### Background

Triple-negative breast cancer (TNBC) is a heterogeneous disease which still lacks defined molecular biomarkers. In the last decade, targeting of specific gene/protein molecular signature of tumors emerged among the best anticancer strategies. Recently, increased expression of the Sortilin (SORT1) receptor has been reported in TNBC patients. Given SORT1 functions in protein internalization, sorting and trafficking, we developed a peptide-anticancer drug conjugation platform to target SORT1-positive breast cancer by linking Docetaxel to a peptide (KA-peptide) that specifically targets SORT1.

### Methods

MDA-MB-231 cells were used as a TNBC cell model for in vitro and in vivo xenograft (CD1 nude mice) assays. Cell migration was assessed using the ×CELLigence real-time system, whereas MTT assay was used for cell proliferation analysis. Apoptosis biomarkers expression was assessed by immunoblotting.

### Results

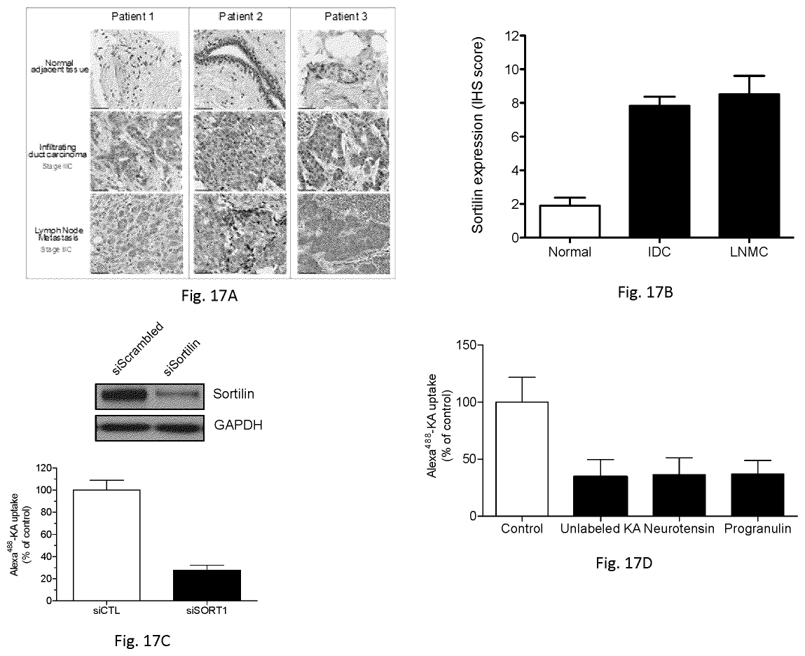

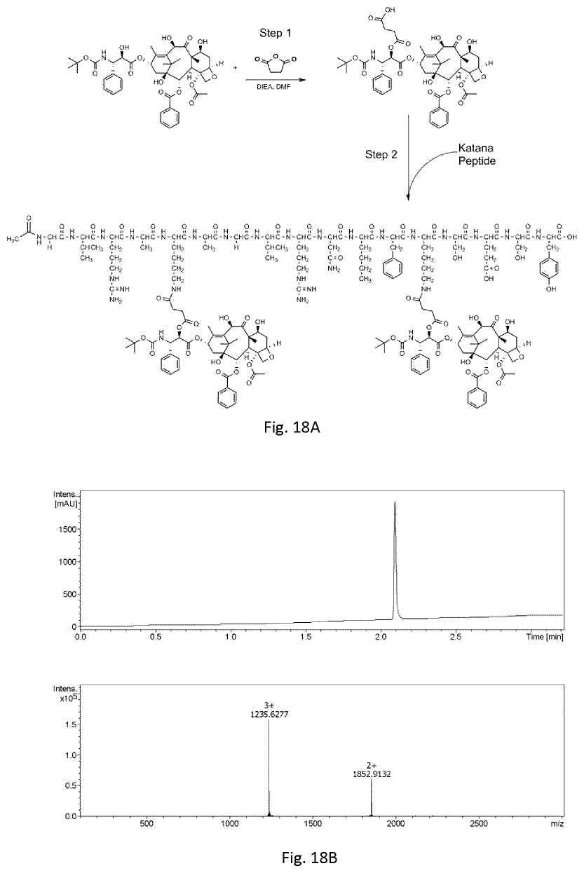

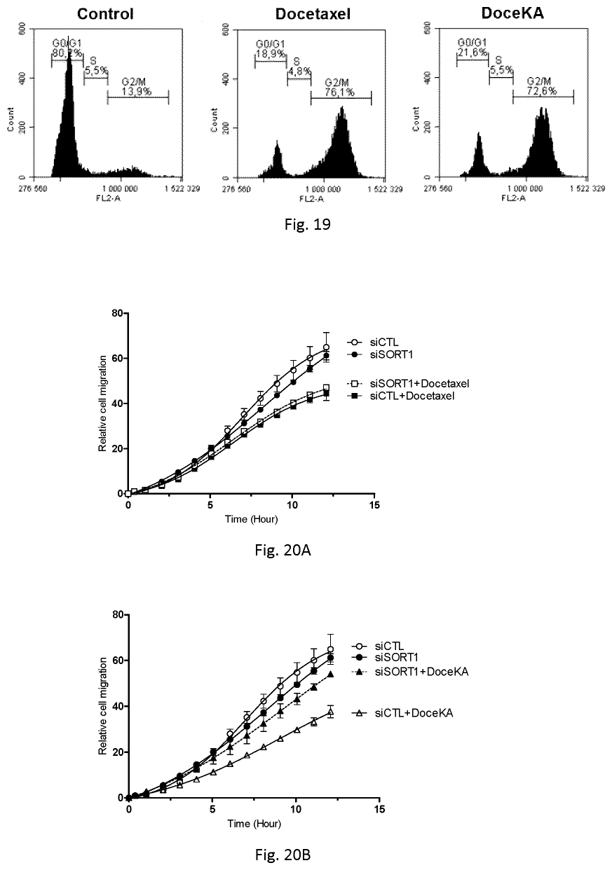

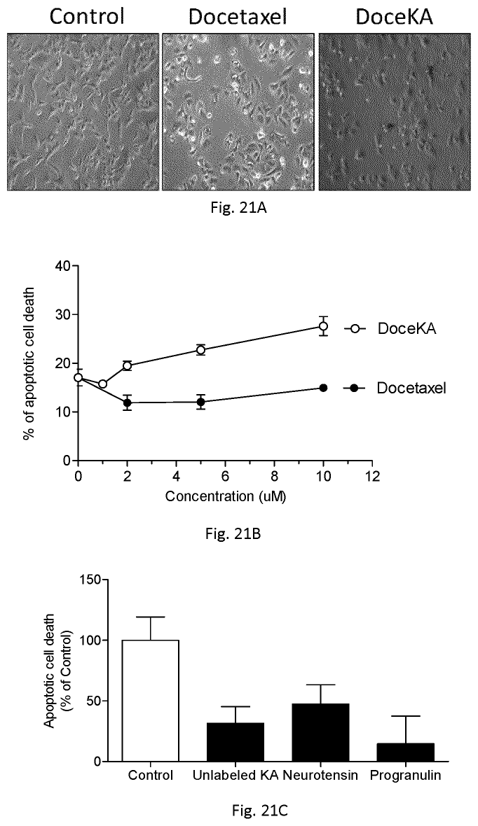

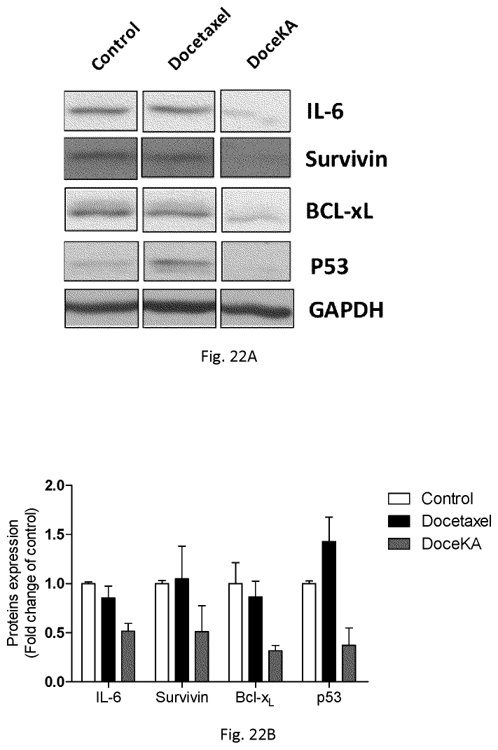

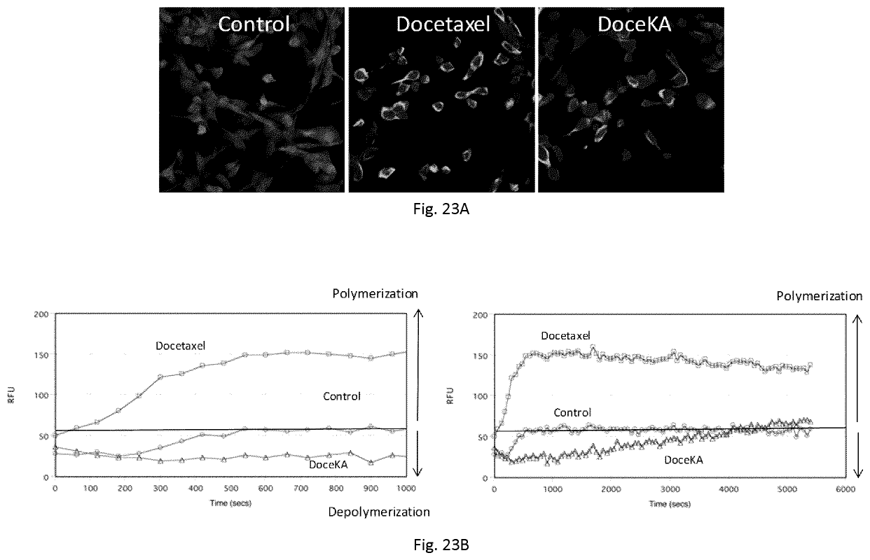

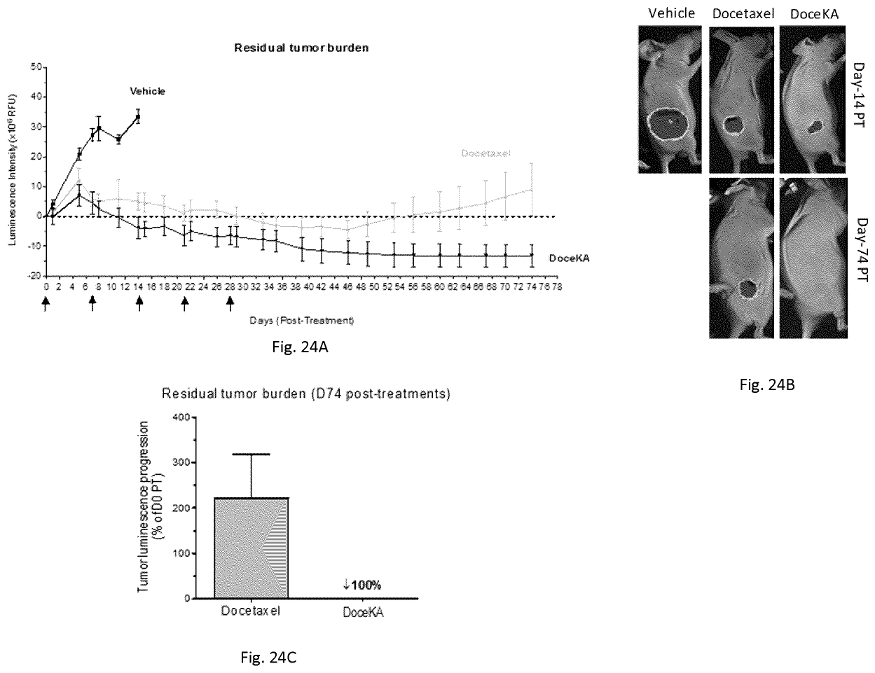

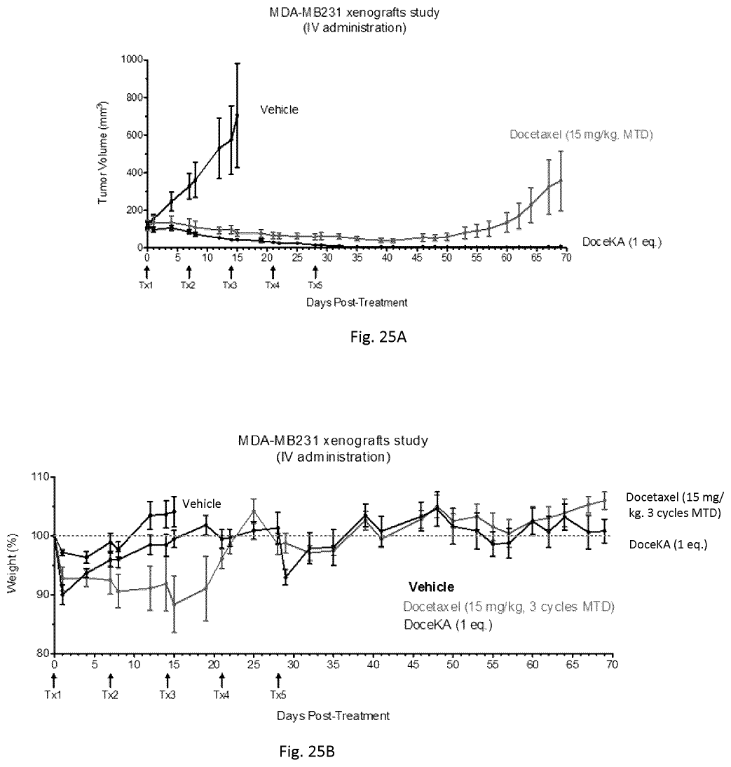

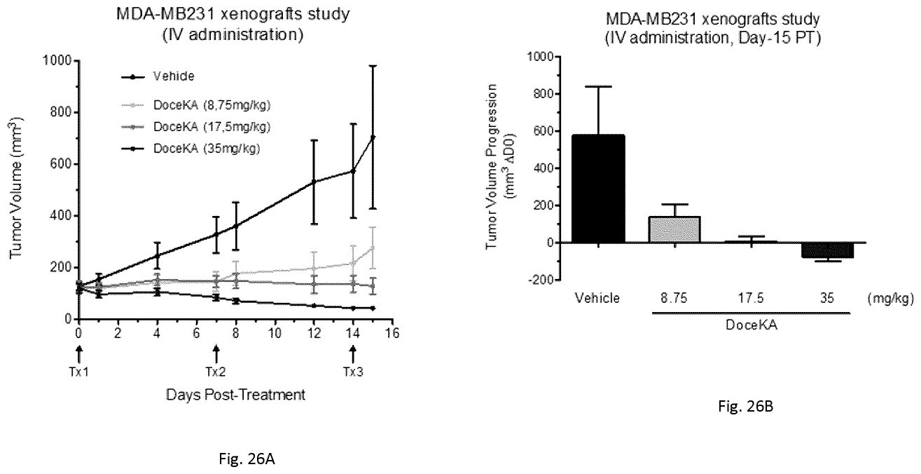

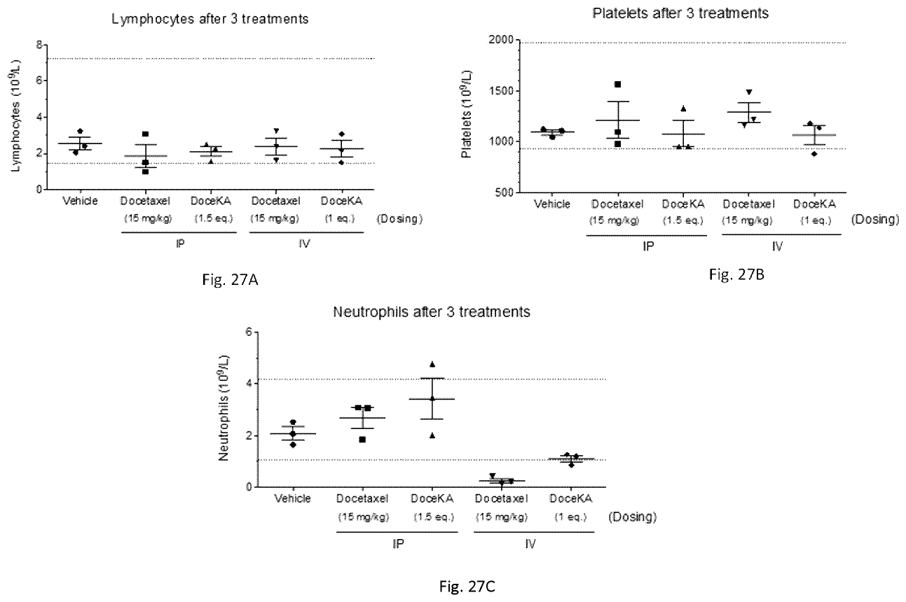

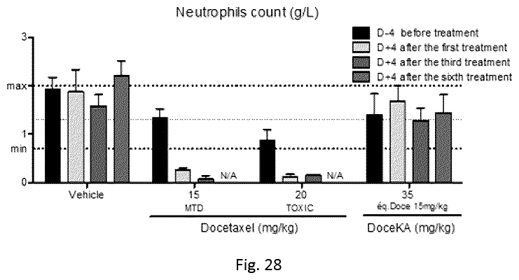

In MDA-MB-231 cells, the Docetaxel-KA-peptide conjugate (DoceKA) KBA-106 (represented by formula Acetyl-GVRAK(docetaxel)AGVRN(Nle)FK(docetaxel)SESY—Formula (XXIII)) exerted potent anti-proliferative and anti-migratory activities in vitro. Results of the experiments are shown in FIGS. 17 to 29. DoceKA triggered faster and higher cell death mechanisms than did free Docetaxel alone. The apoptotic and anti-migratory effects were reversed by the SORT1 ligands Neurotensin and Progranulin, and upon siRNA-mediated silencing of SORT1. DoceKA altered microtubules polymerization and triggered the down-regulation of IL-6, Survivin, Bcl-xL and mutant p53 pro-survival biomarkers (see in particular FIG. 22). In vivo, DoceKA exhibited a greater tumor regression capacity with a prolonged survival in a murine MDA-MB-231 xenograft tumor model than did Docetaxel.

Pharmacokinetic (PK) parameters of Docetaxel compared to conjugated Docetaxe (DoceKA)_are shown in Table 2 below.

TABLE 2
Comparison between pharmacokinetic parameters
of docetaxel and conjugated docetaxel (DoceKA)

| | Docetaxel | DoceKA |
|---|---|---|
| Parameters | (10 mg/kg) | (50 mg/kg) |
| Cmax (μg/ml) | 9.76 | 1617 |
| Cmax (μmol/ml) | 12.5 | 437 |
| AUC (μg/L*h) | 4.8-17 | 2276 |
| AUC (μmol/L*h) | 6-21 | 614 |
| t½ adsorption distribution (min) | 7 | 6-20 |
| t½ elimination (hr) | 1.2 | 1.5-2.5 |
| CL (mL/h/kg) | 2100 | 18 |
| Vd (L/Kg) | 2.2-5 | 0.04 |

PK data on Docetaxel were obtained from refs. 50 and 51. PK analysis for DoceKA was carried out using PK solutions software. Docetaxel PK in mice is biphasic and linear between 13-62 mg/kg. Cmax is about 18-fold higher for DoceKA compared to Docetaxel (20 mg/kg). The area under the curve (AUC) (tissue exposure) is about 29-fold higher for DoceKA indicating higher tissue exposure for DoceKA compared to Docetaxel. Both have similar half-lives. DoceKA has a lower clearance (CL) and Volume of distribution (Vd). These preliminary data need to be confirmed with more data (shorter and longer time points).

### Conclusions

Collectively, the results demonstrated that DoceKA is specifically internalized through a receptor-mediated mechanism. Such property allows for targeting SORT1-positive breast cancers, and makes DoceKA a promising novel therapy for the treatment of TNBC.

### Example 5: Overexpression of SORT1 in Various Cancers

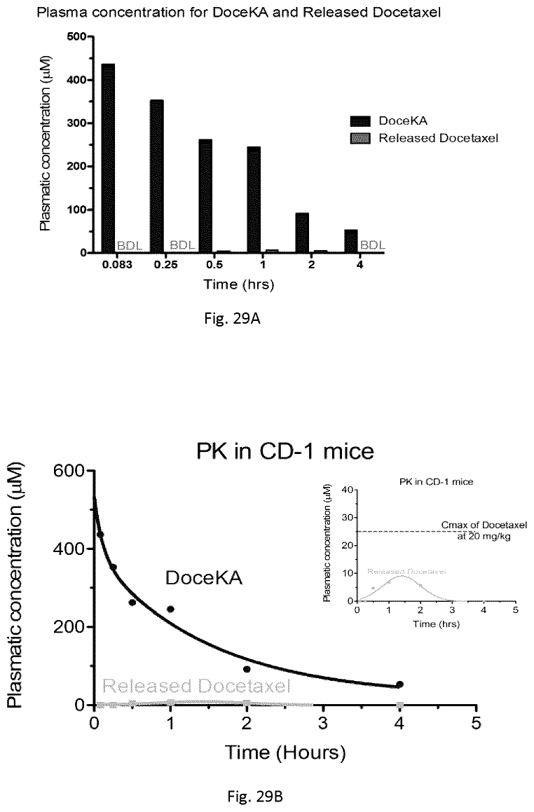

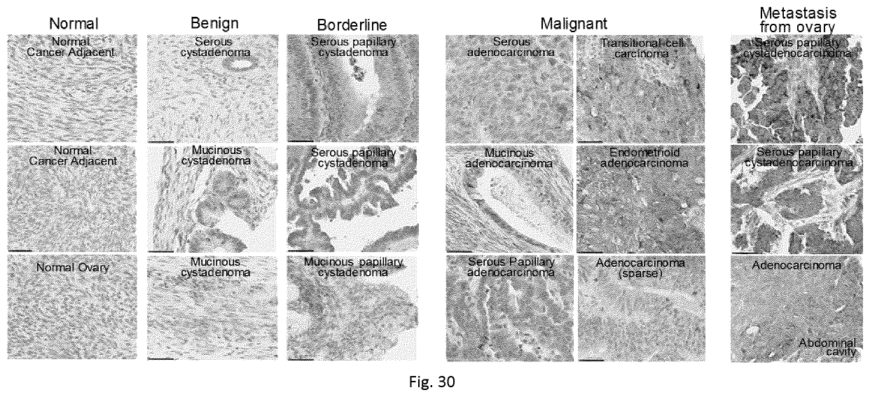

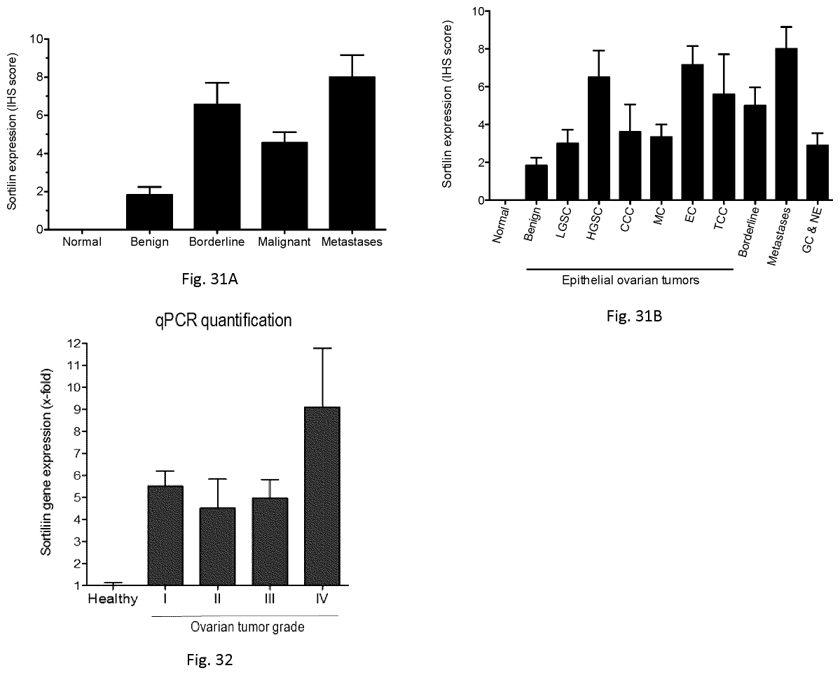

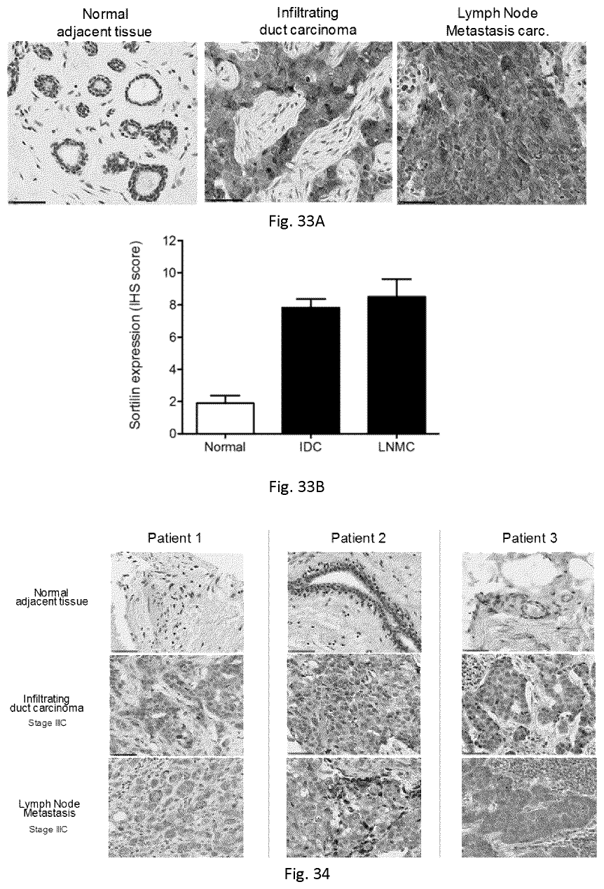

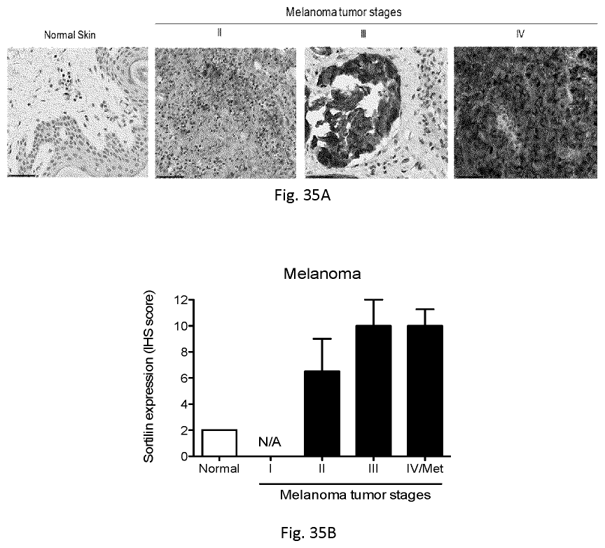

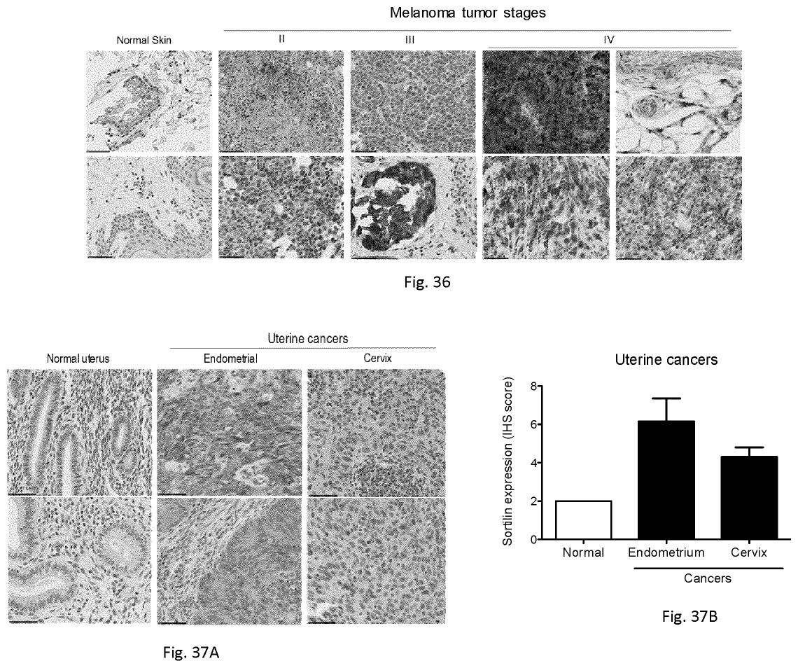

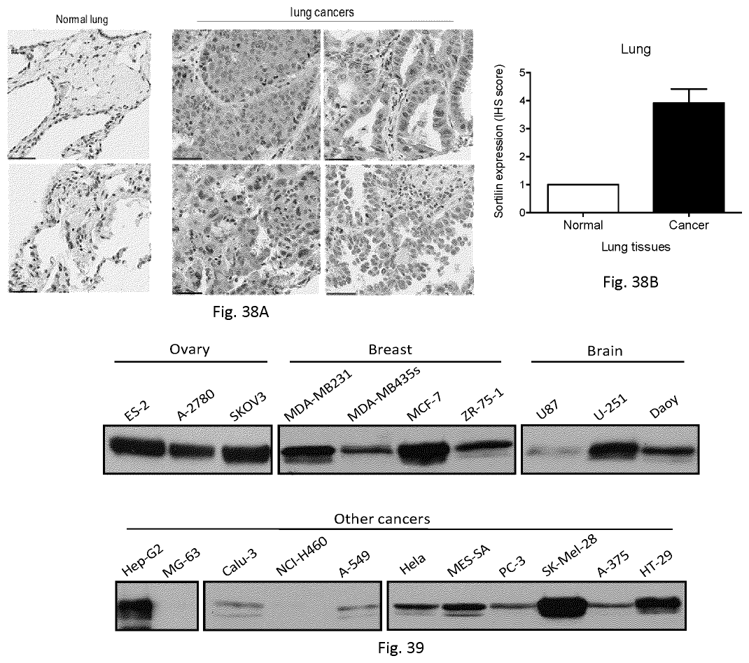

In FIGS. 30 to 40, it is shown that sortilin is overexpressed in a variety of cancers, pathologic subtypes and cancer subtypes, including for example ovarian (e.g. epithelial ovarian), breast (e.g. invasive ductal carcinoma, invasive lobular carcinoma, luminal A, luminal B, HER2+, TNBC), brain, melanoma, uterine (e.g. endometrial and cervix), and lung cancers. An association between SORT1 expression and clinic-pathological parameters in breast cancer has been shown. As shown in Table 3 below.

TABLE 3
Association between SORT1 Expression and Clinico-
Pathological Parameters in Breast Cancer

| | |
|---|---|
| Breast cancer | Sortilin+ |

PATHOLOGIC TYPES

| | |
|---|---|
| Invasive ductal carcinoma | 79% |
| Invasive lobular carcinoma | 54% |

BREAST CANCER SUBTYPES

| | |
|---|---|
| Luminal A | 66% |
| Luminal B | 73% |
| HER2+ | 79% |
| TNBC | 59% |

Sortilin is expressed in normal tissues such as the gastrointestinal tract, pancreas, bone marrow, brain and kidney. HER2 is expression in normal tissues such as liver, gastrointestinal tract, pancreas, bone marrow and breast. All ovarian carcinoma (100%) overexpress Sortilin as compared to 7% for HER2. SORT1 is overexpressed in 66% of breast cancers vs 10-15% for HER2 expression. Both Sortilin and HER2 targets are conserved between species, as shown in Table 4 below:

TABLE 4
Sequence homology compared to human

| | Mouse | Rat |
|---|---|---|
| Sortilin | 91% | 93% |
| Her2 | 89% | 95% |

### Example 6: Role of Sortilin in Vasculogenic Mimicry in Ovarian and Breast Cancers and Inhibition of Vasculogenic Mimicry In Vitro and In Vivo

In FIGS. 40 and 44, it is shown that the Sortilin receptor is involved in early events that lead to the formation of vasculogenic mimicry in ovarian cancer and triple negative breast cancer cells. Sortilin was expressed in the 3D-tubular structures formed by OVCAR-3 and ES-2 ovarian cancer cells (FIGS. 40 and 44). These results indicate that Sortilin positive cells contribute to vasculogenic mimicry in vitro.

Inhibition of Vasculogenic Mimicry by siRNA Gene Silencing

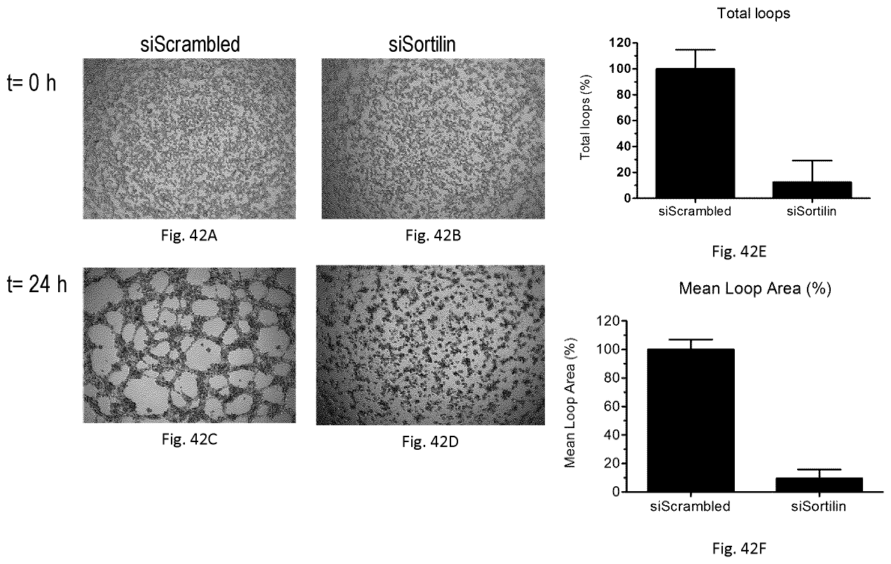

When Sortilin expression was specifically repressed by specific Sortilin siRNA, the 3D-tubular structures were strongly inhibited when compared to scrambled siRNA in MDA-MB231 TNBC cells (FIG. 42A-D). Quantitative analysis of total loops and total tube length was performed as described in previous examples. Results indicate that the number of loops and the total tube length are inhibited by more than 80% upon Sortilin gene silencing (FIGS. 42E and F). These results confirm that Sortilin is important for the formation of 3D-capillary-like structures.

### Inhibition of Vasculogenic Mimicry by Peptide Conjugates

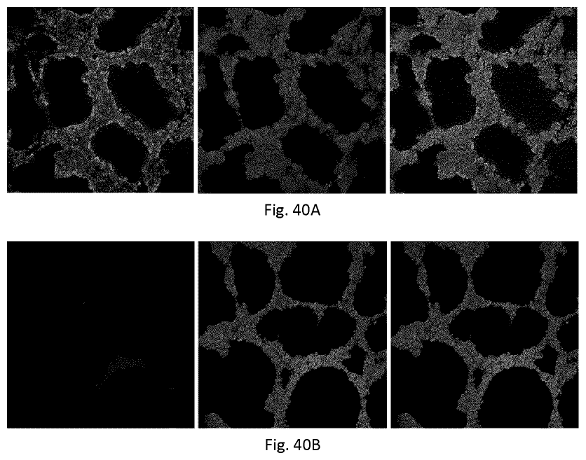

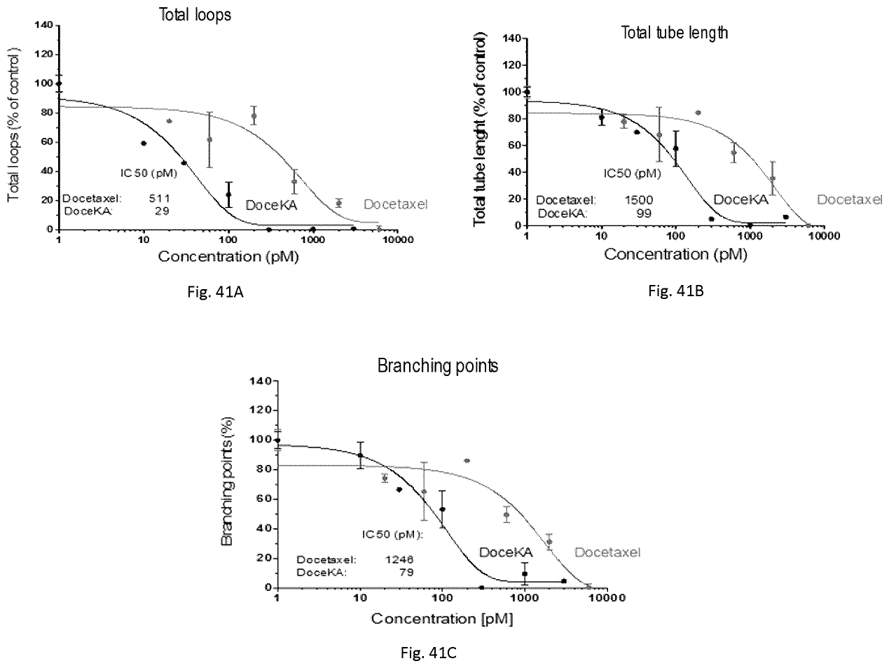

A very low pM concentration of DoceKA inhibits the vasculogenic mimicry of ES-2 ovarian cancer cells (FIG. 41). DoceKA caused a stronger inhibition of vasculogenic mimicry as compared to Docetaxel alone (FIG. 43).

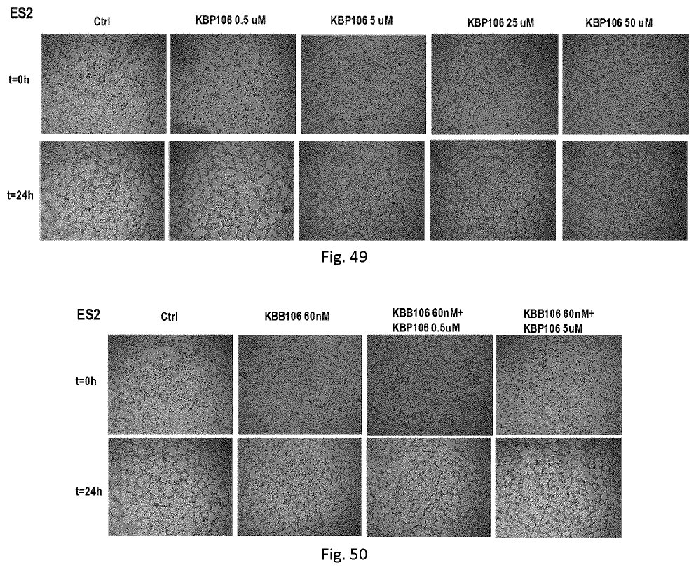

In FIGS. 49 to 50, it is shown that KA-peptide drug conjugates targeting Sortilin exhibited strong anti-vasculogenic mimicry effects through suppressing the invasive phenotype of ovarian and breast cancer cells. Vasculogenic mimicry of ES-2 ovarian cancers was assessed on Matrigel in the presence of the KBP106 peptide or conjugated doxorubicin (KBB106) (FIGS. 49 and 50). Under the experimental conditions used for the Katana conjugates, the peptide (KBP106) has no significant effect on vasculogenic mimicry up to 50 μM (FIG. 49). Despite the fact the peptide alone (KBP106) has no effect on vasculogenic mimicry, the addition of the peptide to KBB106 prevents the vasculogenic mimicry inhibition of Doxorubicin conjugate (KBB106) (FIG. 50). These results suggest that KBP106, by binding to Sortilin, prevents the interaction of KBB106 with the receptor.

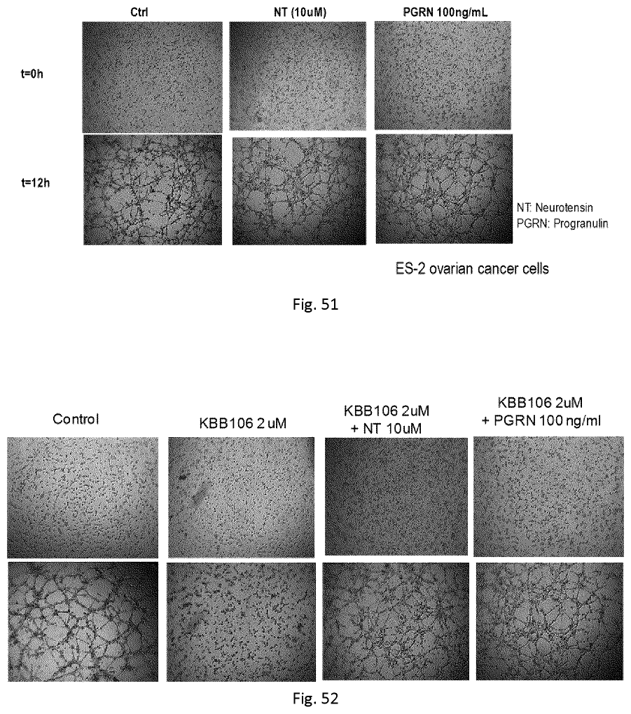

In FIGS. 51 to 52, it is shown that in ES-2 ovarian cancer cells, Sortilin ligands, neurotensin and progranulin, do not effect vasculogenic mimicry (FIG. 51), but do reverse vasculogenic mimicry inhibition caused by cell exposure to KBB106.

### Inhibition of Vasculogenic Mimicry by Anti-Sortilin mAb

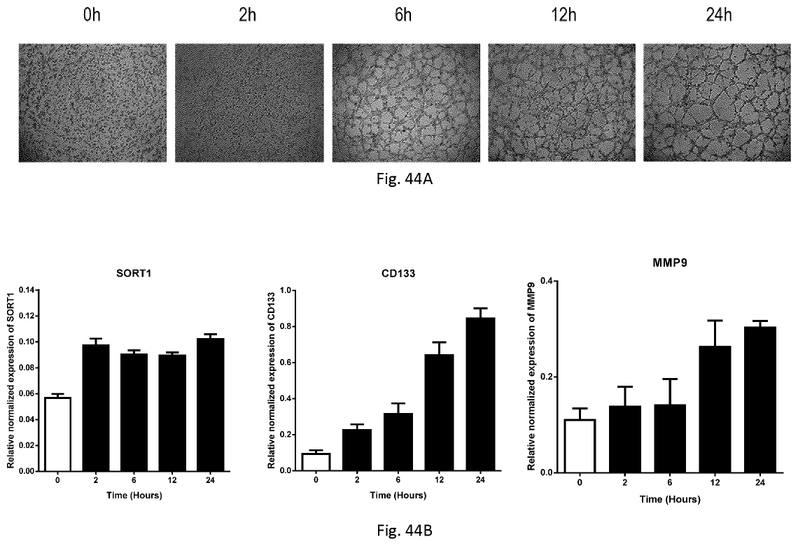

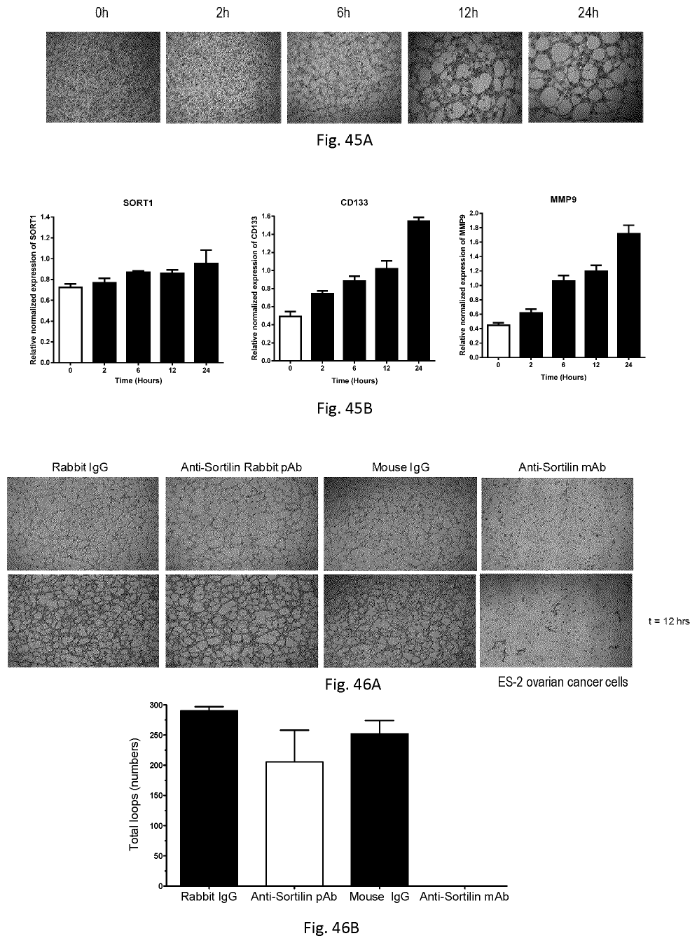

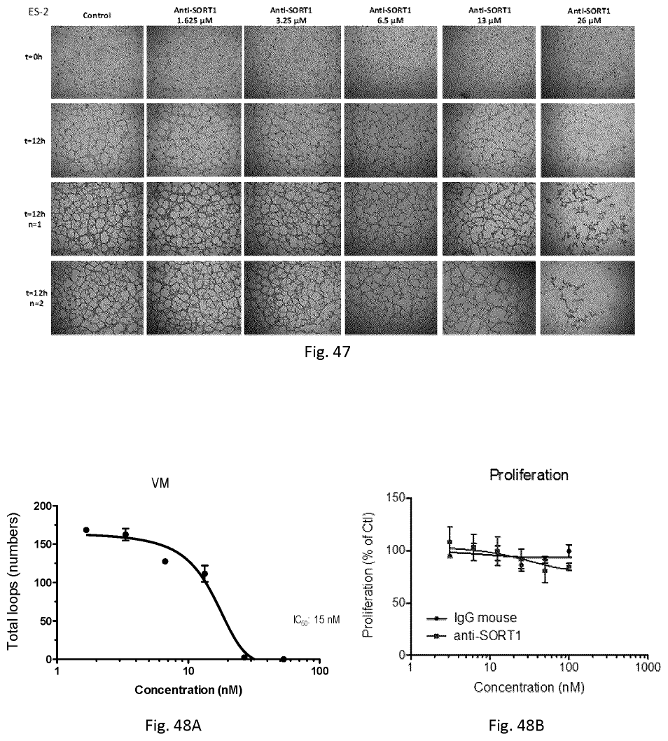

In FIGS. 46 to 48, it is shown that vasculogenic mimicry formation was mostly abolished by anti-Sortilin mAb. ES-2 ovarian cancer cells were seeded on top of Matrigel in the presence of either a non-specific mouse IgG, Rabbit IgG, an anti-Sortilin Rabbit pAb, or an anti-Sortilin mAb (FIG. 46). Results show that incubation with the anti-Sortilin mAb prevented the formation of 3D-tubular structures in ES-2 ovarian cancer, whereas the other antibodies tested had no effect (FIG. 46). ES-2 ovarian cancer cells were exposed to increasing concentrations of Anti-Sort1, which demonstrated that increasing concentrations of Anti-Sort1 are more effective at preventing the formation of 3D-tubular structures than lesser concentrations (FIG. 47). At concentrations which inhibit vasculogenic mimicry (12-24 hrs), anti-Sortilin has no effect on ES-2 cancer cell proliferation indicating that vasculogenic mimicry inhibition is not related to a cytotoxic effect (FIG. 48). Table 5 below shows the effect of various concentrations of Anti-Sort1 on tube length, branching and number of loops in ES-2 ovarian cancer cells.

TABLE 5
The effect of various concentrations of Anti-Sort1 on tube length,
branching and number of loops present during vasculogenic mimicry

| | | | SORT | SORT | SORT | SORT |
|---|---|---|---|---|---|---|
| | Ctrl | Ctrl | 0.25 μg/mL | 0.25 μg/mL | 0.5 μg/mL | 0.5 μg/mL |
| Sample name | (n = 1) | (n = 2) | (n = 1) | (n = 2) | (n = 1) | (n = 2) |
| Total Tube Length | 34102 | 32769 | 33065 | 33384 | 33558 | 33281 |
| (pixels) | | | | | | |
| Total Branching Points | 455 | 417 | 408 | 418 | 411 | 431 |
| Total Loops | 173 | 160 | 171 | 166 | 168 | 157 |

| | SORT | SORT | SORT | SORT | SORT | SORT |
|---|---|---|---|---|---|---|
| | 1 μg/mL | 1 μg/mL | 2 μg/mL | 2 μg/mL | 4 μg/mL | 2 μg/mL |
| Sample name | (n = 1) | (n = 2) | (n = 1) | (n = 2) | (n = 1) | (n = 2) |
| Total Tube Length | 29793 | 30395 | 28759 | 27470 | 11887 | 4307 |
| (pixels) | | | | | | |
| Total Branching Points | 350 | 360 | 324 | 321 | 148 | 65 |
| Total Loops | 129 | 126 | 104 | 119 | 5 | 0 |

### Example 7: Expression of Sortilin and CD133 During Formation of Vasculogenic Mimicry

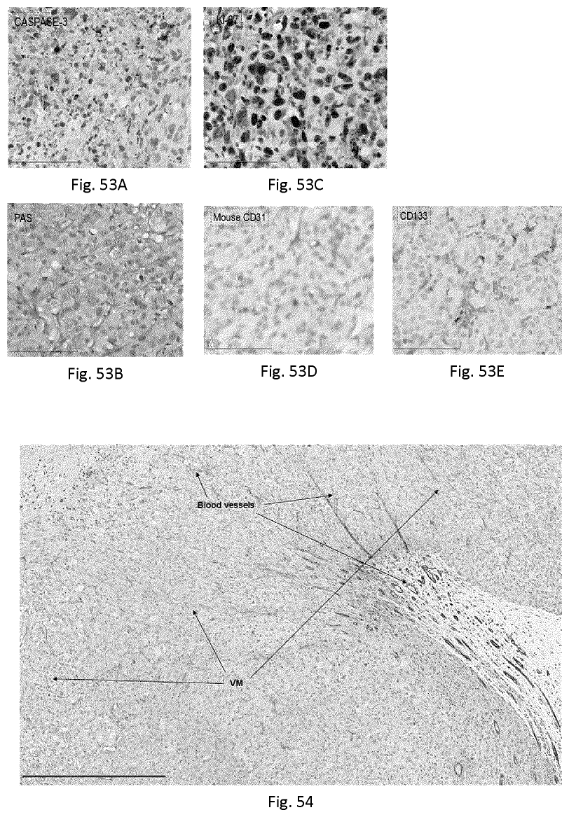

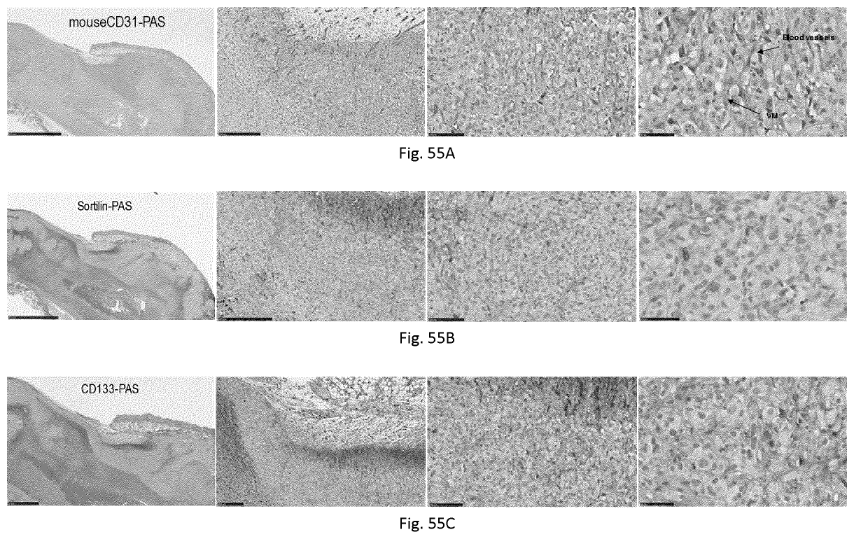

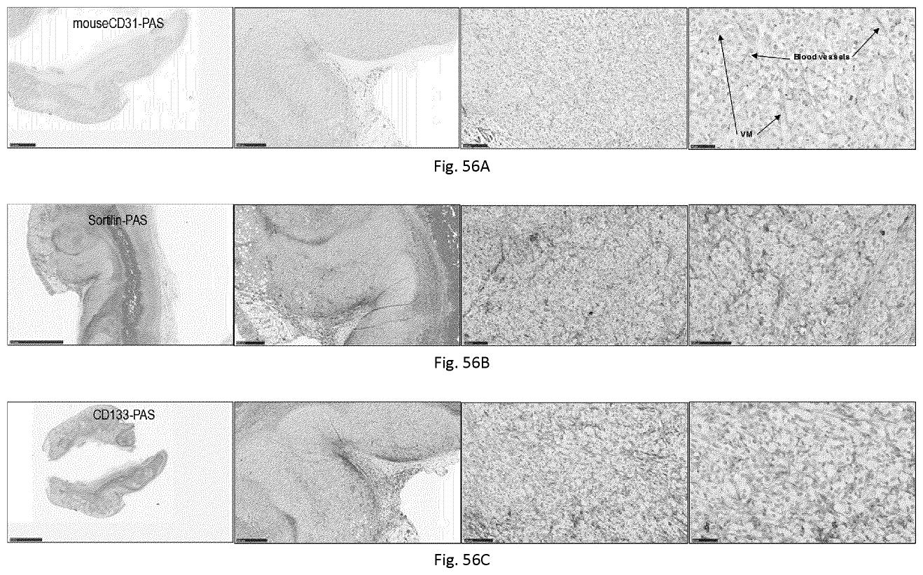

In FIGS. 53 to 57, it is shown that both Sortilin and CD133 are present in vasculogenic mimicry structures formed by ES-2 tumor xenografts. Tumor tissue sections were first hybridized with primary antibodies against Sortilin, CD31, and CD133 or stained with periodic acid Schiff (PAS) (FIG. 53). Then the sections were co-stained with either PAS-anti-CD31, PAS-anti-Sortilin or PAS-anti-CD133 (FIGS. 54 to 57). The vasculogenic mimicry structures observed were PAS positive and CD31 negative and are indicated by word “VM”, whereas blood vessels are indicated by the words “blood vessels” and are positive for CD31. Both Sortilin and CD133 stainings were associated with cancer cells and also in part with PAS staining.

Vasculogenic mimicry identification by immunohistochemistry was carried as follows. Formalin-fixed, paraffin-embedded tumor tissue sections were stained with the appropriate primary antibodies to detect CD31, CD133 and Sortilin. Then the sections were treated with 0.5% periodic acid solution for 15 min. After rinsing with distilled water for 2 min, the tissue sections were placed into Schiff solution for 15-30 min in a dark chamber then rinsed with distilled water for 3 times. Afterwards, sections were counterstained with hematoxylin. 3D-tubular structures were seen to be formed by CD31-negative tumor cells in hematoxylin-eosin staining slides. Then, vasculogenic mimicry was validated by CD31/periodic acid-Schiff (PAS) double-staining and it was identified by the detection of PAS-positive loops surrounded with tumor cells (not endothelial cells). With respect to immunohistochemical staining for CD133 and Sortilin, formalin-fixed, and paraffin-embedded slides from cancer tissue sections were deparaffinized and rehydrated conventionally. Afterwards, slides were incubated with 3% H₂O₂ for blocking the endogenous peroxidase, and then with 20% goat serum for reducing nonspecific binding. Mouse anti-human CD133 monoclonal antibodies (clone CD133, Miltenyi Biotec) were added to the slides at a dilution of 1:50 after washing with phosphate buffer saline. Then slides were incubated at 4° C. overnight. Next day, slides were incubated in peroxidase-conjugated rabbit anti mouse secondary antibodies (DakoCytomation, Carpinteria, Calif., USA) for 30 min at 1:200. Diaminobenzidine was used for visualizing the reactions. Finally, the slides were counterstained with hematoxylin.

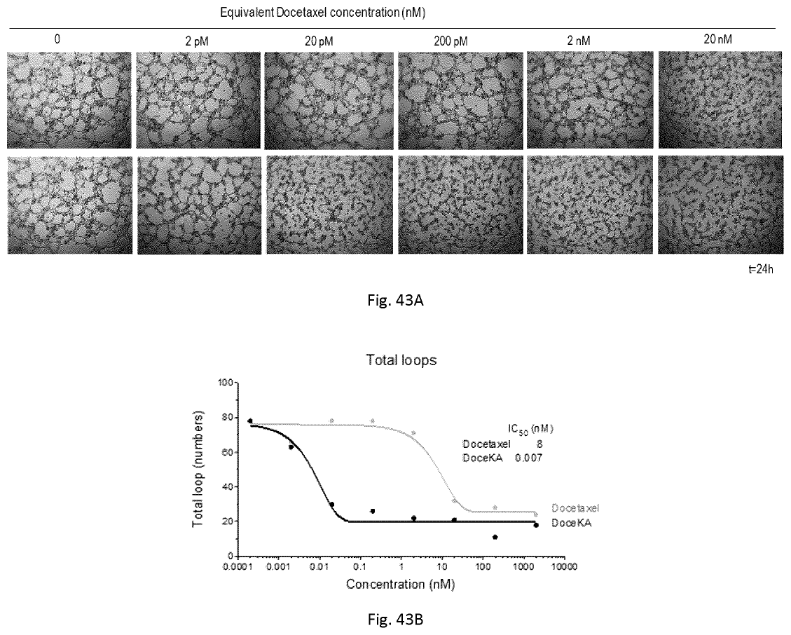

In FIGS. 44 and 45, it is shown that the expression of Sortilin and of cancer stem-like CD133 genes increased during the formation of vasculogenic mimicry 3D-tubular structures.

Gene expression of Sortilin, CD133, and MMP9 was assessed by reverse transcription and quantitative polymerase chain reaction (RT-qPCR) during the formation of 3D-tubular structures by ES-2 ovarian cancer cells (FIG. 44) and MDA-MB-231 cells (FIG. 45). During the formation of these capillary-like structures by ES-2 ovarian cancer cells, Sortilin gene expression is rapidly increased after 2 hrs and remained high for 24 hours (FIG. 44). CD133 and MMP9 gene expression increased by more than 4-fold overtime during vasculogenic mimicry formation. CD133 is one of the most commonly used markers for isolation of cancer stem cells (CSC) population from tumors. CD133+ cancer cells (CSC) were positively associated with vasculogenic mimicry formation, local regional recurrence and distant metastasis. In the case of vasculogenic mimicry in MDA-MB-231 (FIG. 45), Sortilin and CD133 gene expression slightly increased overtime and MMP9 by about 2-fold. This suggests that in MDA-MB-231, contribution of CD133 in vasculogenic mimicry may be modest as opposed to ES-2 cancer cells were a much stronger increased in CD133 gene expression was observed.

### Example 8: Screening of Additional Sortilin Positive TNBC Line Models

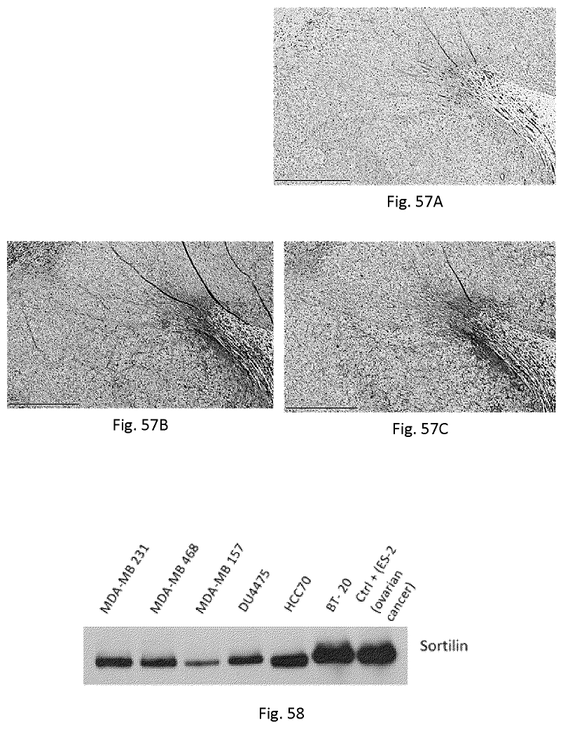

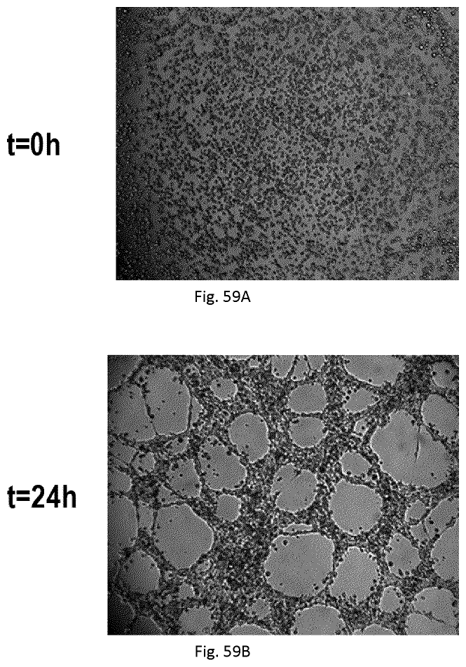

Additional TNBC cell lines expression sortilin are being screened. In FIGS. 58 to 59, it is shown that various triple negative breast cancer (TNBC) cell lines are Sortilin positive cell lines (FIG. 58 and Table 6), and one line in particular has been confirmed to form 3D-tubular vasculogenic mimicry structures, BT-20 TNBC cells (FIG. 59).

TABLE 6
List of TNBC cell line models

| Type | Subtype | Name | Histology |
|---|---|---|---|
| Basal-Like | BL-1 | HCC1599 | Primary ductal carcinoma |
| Morphology | | HCC1937 | Primary ductal carcinoma |
| | | HCC1143 | Primary ductal carcinoma |
| | | MDA-MB468 | Adenocarcinoma |
| | | HCC38 | Primary ductal carcinoma |
| | BL-2 | HCC70 | Primary ductal carcinoma |
| | | HCC1806 | Primary acantholytic |
| | | | squamous cell carcinoma |
| | IM | HCC1187 | Primary ductal carcinoma |
| | | DU4475 | Carcinoma |
| Mesenchymal- | M | BT-549 | Ductal carcinoma |
| Like | MSL | Hs578T | Carcinoma |
| Morphology | | MDA-MB231 | Adenocarcinoma |
| | | MDA-MB436 | Adenocarcinoma |
| | | MDA-MB157 | Medulallary carcinoma |
| LAR | LAR | MDA-MB453 | Carcinoma |
| Unclassified | | BT-20 | Carcinoma |
| Morphology | | HCC1395 | Primary ductal carcinoma |

The embodiments of paragraphs [0062] to [00458] of the present disclosure are presented in such a manner in the present disclosure so as to demonstrate that every combination of embodiments, when applicable, can be made. These embodiments have thus been presented in the description in a manner equivalent to making dependent claims for all the embodiments that depend upon any of the preceding claims (covering the previously presented embodiments), thereby demonstrating that they can be combined together in all possible manners. For example, all the possible combinations, when applicable, between the embodiments of paragraphs [0062] to [00458] and the various aspects presented in paragraphs [005] to [0064] are hereby covered by the present disclosure.

### REFERENCES

1. World Cancer Report, 2014.
2. Urruticoechea A, Alemany R, Balart J, Villanueva A, Viñals F, Capellá G. Recent advances in cancer therapy: an overview. Curr Pharm Des 2010; 16:3-10.
3. Fisher R, Pusztai L, Swanton C. Cancer heterogeneity: implications for targeted therapeutics. Brit J Cancer 2013; 108; 479-485.
4. Zhao P, Astruc D. Docetaxel nanotechnology in anticancer therapy. Chem Med Chem 2012; 7:952-72.
5. Ophir E, Bobisse S, Coukos G, Harar A, Kandalaft L E. Personalized approaches to active immunotherapy in cancer. Biochim Biophys Acta 2016; 1865:72-82.
6. Sapiezynski J, Taratula O, Rodriguez-Rodriguez L, Minko T. Precision targeted therapy of ovarian cancer. J Cont Rel 2016; 243:250-268.
7. Wilson C M, Naves T, Saada S, Pinet S, Vincent F, LallouéF, Jauberteau M O. The implications of sortilin/vps10p domain receptors in neurological and human diseases. CNS Neurol Disord Drug Targets 2014; 13:1354-65.
8. Vincent J P, Mazella J, Kitabgi P. Neurotensin and neurotensin receptors. Trends Pharmacol Sci 1999; 20:302-309.
9. Carlo A S, Nykjaer A, Willnow T E. Sorting receptor sortilin-a culprit in cardiovascular and neurological diseases. J Mol Med 2014; 92:905-11.
10. Schmidt V, Willnow T E. Protein sorting gone wrong-VPS10P domain receptors in cardiovascular and metabolic diseases. Atheroscler 2016; 245:194-9.
11. Wilson C M, Naves T, Ai Akhrass H, Vincent F, Melloni B, Bonnaud F, Lalloué F, Jauberteau M O. A new role under sortilin's belt in cancer. Com Integr Biol 2016; 9:e1130192.
12. Al-Shawi R, Hafner A, Chun S, Raza S, Crutcher K, Thrasivoulou C, Simons P, Cowen T. ProNGF, sortilin, and age-related neurodegeneration. Ann N Y Acad Sci 2007; 1119:208-15.
13. Lewin G R, Nykjaer A. Pro-neurotrophins, sortilin, and nociception. Eur J Neurosci 2014; 39:363-74.
14. Mazella J, Vincent J P. Internalization and recycling properties of neurotensin receptors. Peptides 2006; 27:2488-92.
15. Vaegter C B, Jansen P, Fjorback A W, Glerup S, Skeldal S, Kjolby M, Richner M, Erdmann B, Nyengaard J R, Tessarollo L, et al. Sortilin associates with Trk receptors to enhance anterograde transport and neurotrophin signaling. Nat Neurosci 2011; 14:54-61.
16. Akil H, Perraud A, Melin C, Jauberteau M O, Mathonnet M. Fine-tuning roles of endogenous brain-derived neurotrophic factor, TrkB and sortilin in colorectal cancer cell survival. PloS One 2011; 6:e25097.
17. Dal Farra C, Sarret P, Navarro V, Botto J M, Mazella J, Vincent J P. Involvement of the neurotensin receptor subtype NTR3 in the growth effect of neurotensin on cancer cell lines. Int J Cancer 2001; 92:503-9.
18. Truzzi F, Marconi A, Lotti R, Dallaglio K, French L E, Hempstead B L, Pincelli C. Neurotrophins and their receptors stimulate melanoma cell proliferation and migration. J Invest Dermatol 2008; 128:2031-40.
19. Giorgi R R, Chile T, Bello A R, Reyes R, Fortes M A, Machado M C, Cescato V A, Musolino N R, Bronstein M D, Giannella-Neto D, et al. Expression of neurotensin and its receptors in pituitary adenomas. J Neuroendocrinol 2008; 20:1052-7.
20. Xiong J, Zhou L, Yang M, Lim Y, Zhu Y H, Fu D L, Li Z W, Zhong J H, Xiao Z C, Zhou X F. ProBDNF and its receptors are upregulated in glioma and inhibit the growth of glioma cells in vitro. Neuro Oncol 2013; 15:990-1007.
21. Hemmati S, Zamani A H, Mahmoudi A R, Sadeghi M R, Soltanghoraee H, Akhondi M M, Tarahomi M, Jeddi-Tehrani M, Rabbani H. Ectopic Expression of Sortilin 1 (NTR-3) in Patients with Ovarian Carcinoma. Avicenna J Med Biotechnol 2009; 1:125-31.
22. Ghaemimanesh F, Ahmadian G, Talebi S, Zamani A H, Behmanesh M, Hemmati S, Hadavi R, Jeddi-Tehrani M, Farzi M, Akhondi M M, Rabbani H. The effect of sortilin silencing on ovarian carcinoma cells. Avicenna J Med Biotechnol 2014; 6:169-77.
23. Hosseini A, Ghorbani A. Cancer therapy with phytochemicals: evidence from clinical studies. Avicenna J Phytomed. 2015; 5:84-97.
24. Cao Z, Bao M, Miele L, Sarkar F H, Wang Z, Zhou Q. Tumour vasculogenic mimicry is associated with poor prognosis of human cancer patients: a systemic review and meta-analysis. Eur J Cancer. 2013 49:3914-23.
25. Kirschmann D A, Seftor E A, Hardy K M, Seftor R E, Hendrix M J. Molecular pathways: vasculogenic mimicry in tumor cells: diagnostic and therapeutic implications. Clin Cancer Res. 2012; 18:2726-32.
26. Clarjs R, Otte-Holler I, Ruiter D J, de Waal R M. Presence of a fluid-conducting meshwork in xenografted cutaneous and primary human uveal melanoma. Invest Ophthalmol Vis Sci. 2002; 43:912-8.
27. Kobayashi H, Shirakawa K, Kawamoto S, Saga T, Sato N, Hiraga A, Watanabe 1, Heike Y, Togashi K, Konishi J, Brechbiel M W, Wakasugi H. Rapid accumulation and internalization of radiolabeled herceptin in an inflammatory breast cancer xenograft with vasculogenic mimicry predicted by the contrast-enhanced dynamic MRI with the macromolecular contrast agent G6-(1B4M-Gd)(256). Cancer Res. 2002; 62:860-6.
28. Maniotis A J1, Chen X, Garcia C, DeChristopher P J, Wu D, Pe'er J, Folberg R. Control of melanoma morphogenesis, endothelial survival, and perfusion by extracellular matrix. Lab Invest. 2002; 82:1031-43.
29. Qiao L, Liang N, Zhang J, Xie J, Liu F, Xu D, Yu X, Tian Y. Advanced research on vascuiogenic mimicry in cancer. J Cell Mol Med. 2015; 19:315-26.
30. Liu T J, Sun B C, Zhao X L, Zhao X M, Sun T, Gu Q, Yao Z, Dong X Y, Zhao N, Liu N. CD133+ cells with cancer stem cell characteristics associates with vasculogenic mimicry in triple-negative breast cancer. Oncogene. 2013; 32:544-53.
31. Racordon D, Valdivia A, Mingo G, Erices R, Aravena R, Santoro F, Bravo M L, Ramirez C, Gonzalez P, Sandoval A, GonzBlez A, Retamal C, Kogan M J, Kato S, Cuello M A, Osorio G, Nualart F, Alvares P, Gago-Arias A, Fabri D, Espinoza 1, Sanchez B, Corvalán A H, Pinto M P, Owen G I. Structural and functional identification of vasculogenic mimicry in vitro. Sci Rep. 2017; 7:6985.
32. Chiablaem K, Lirdprapamongkol K, Keeratichamroen S, Surarit R, Svasti J. Curcumin suppresses vasculogenic mimicry capacity of hepatocellular carcinoma cells through STAT3 and PI3K/AKT inhibition. Anticancer Res. 2014; 34:1857-64.
33. McCafferty J, Griffiths A D, Winter G, Chiswell D J. Phage antibodies: filamentous phage displaying antibody variable domains. Nature 1990; 348: 552-554.
34. Carter P, Merchant A M. Engineering antibodies for imaging and therapy. Current Opinion in Biotechnology 1997; 8:449-454.
35. Riechmann L, Clark M, Waldmann H, Winter G. Reshaping human antibodies for therapy. Nature 1988; 332:323-327.
36. Foote J, Winter G. Antibody framework residues affecting the conformation of the hypervariable loops. Journal of Molecular Biology 1992; 224:487-499.
37. Yano S, Hsu R K, Landolfi N F, Vasquez M, Cole M, Tso J T, Bringman T, Laird W, Hudson D. A humanized antibody specific for the platelet integrin gpIIb/IIIa. The Journal of Immunology 1994; 152:2968-2976.
38. Pedersen J T, Henry A H, Searle S J, Guild B C, Roguska M, Rees A R. Comparison of surface accessible residues in human and murine immunoglobulin Fv domains: implication for humanization of murine antibodies. Journal of molecular biology 1994; 235:959-973.
39. Köhler G, Milstein C. Continuous cultures of fused cells secreting antibody of predefined specificity. Nature 1975, 256: 495-497.
40. Kozbor D, Roder J C. The production of monoclonal antibodies from human lymphocytes. Immunology Today 1983, 4: 72-79.
41. Cole S P C, Kozbor D, Roder J C. The EBV-hybridoma technique and its application to human lung cancer. In: Reisfeld R A, Sell S, eds. Monoclonal Antibodies and Cancer Therapy. New York: Alan R. Liss Inc. (UCLA Symposia on Molecular and Cellular Biology) 1985, 27:77-96.
42. Ge H, Luo H. Overview of advances in vasculogenic mimicry—a potential target for tumor therapy. Cancer Manag Res. 2018; 10:2429-2437.
43. Zhou Q, Zhifei C, Meimei B, Miele L, Sarkar F H, Wang Z. Tumour vasculogenic mimicry is associated with poor prognosis of human cancer patients: A systemic review and meta-analysis. Eur J Cancer 2013; 49: 3914-3923.
44. Yang, J. P. et al. Tumor vasculogenic mimicry predicts poor prognosis in cancer patients: a meta-analysis. Angiogenesis 2016; 19: 191-200.
45. Sun B, Zhang D, Zhao N and Zhao X. Epithelial-to-endothelial transition and cancer stem cells: two cornerstones of vasculogenic mimicry in malignant tumors. Oncotarget 2017; 8: 30502-30510.
46. Liang J, Yang B, Cao Q, Wu X. Association of Vasculogenic Mimicry Formation and CD133 Expression with Poor Prognosis in Ovarian Cancer. Gynecol Obstet Invest. 2016; 81:529-536.
47. Altschul et al., 1997, Nucleic Acids Res. 25:3389-3402.
48. Navarro et al., 2002.
49. Altschul et al., 1990, J. Mol. Biol. 215:403.
50. Bissery et al. 1995.
51. Clarke et al. 1999.
52. Karlin and Altschul, 1990, Proc. Natl. Acad. Sci. U.S.A. 87:2264-2268.
53. Karlin and Altschul, 1993, Proc. Natl. Acad. Sci. U.S.A. 90:5873-5877.

1-50. (canceled)

51. A method of inhibiting vasculogenic mimicry in cancerous tissues formed by cancerous cells expressing Sortilin comprising administering to a subject in need thereof a therapeutically effective amount of at least one conjugate compound having the formula of A-(B)ₙ,
wherein
n is 1, 2, 3 or 4;
A is a peptide compound having at least 80% sequence identity to a compound chosen from compounds of formula (V), formula (VI), formula (VII), formula (VIII), formula (IX), formula (X), formula (XI), formula (XII) and formula (XIII):
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL
wherein at least one protecting group and/or at least one labelling agent is optionally connected to said peptide compound at an N- and/or C-terminal end; and
B is at least one therapeutic agent, wherein B is connected to A, optionally via a linker,
optionally the peptide compound is cyclic.

52. The method of claim 51
wherein B is connected to A at a free amine of a lysine residue of said peptide compound, optionally via a linker, or at an N-terminal position of said peptide compound, optionally via a linker.

53-65. (canceled)

66. The method of claim 51, wherein the peptide compound has at least 90% sequence identity to the compound chosen from compounds of formula (V), formula (VI), formula (VII), formula (VIII), formula (IX), formula (X), formula (XI), formula (XII) and formula (XIII).

67. The method of claim 51, wherein the peptide compound is represented by formula (X) and consists of the amino acid sequence of SEQ ID NO: 10.

68. The method of claim 51, wherein the peptide compound is represented by formula (XI) and consists of the amino acid sequence of SEQ ID NO: 11.

69. The method of claim 51, wherein the peptide compound comprises at least one protecting group that is acetyl or succinyl.

70. The method of claim 51, wherein the peptide compound is represented by Formula (XXXVIII), Formula (XXXIX), Formula (XL), Formula (XLI) or Formula (XLII):
(XXXVIII)
(SEQ ID NO: 14)
Acetyl-GVRAKAGVRNMFKSESY
(XXXIX)
(SEQ ID NO: 15)
Acetyl-GVRAKAGVRN(Nle)FKSESY
(XL)
(SEQ ID NO: 16)
Acetyl-YKSLRRKAPRWDAPLRDPALRQLL
(XLI)
(SEQ ID NO: 17)
Acetyl-YKSLRRKAPRWDAYLRDPALRQLL
(XLII)
(SEQ ID NO: 18)
Acetyl-YKSLRRKAPRWDAYLRDPALRPLL.

71. The method of claim 51, wherein B is connected to A via a linker, optionally a cleavable linker or a non-cleavable linker.

72. The method of claim 51, wherein the at least one therapeutic agent is a phytochemical agent or an anticancer agent.

73. The method of claim 72, wherein the anticancer agent is docetaxel, doxorubicin, cabazitaxel, maytansinoids, auristatins, calicheamicins, amatoxins, amanitin, or aldoxorubicin.

74. The method of claim 73, wherein the anticancer agent is docetaxel.

75. The method of claim 74, wherein the conjugate compound is represented by formula (XIX) or (XXIII):
GVRAK(docetaxel)AGVRN(Nle)FK(docetaxel)SESY Formula (XIX)
that comprises the peptide compound of SEQ ID NO: 10 wherein each lysine residue has a docetaxel molecule connected thereto; or
Acetyl-GVRAK(docetaxel)AGVRN(Nle)FK(docetaxel)SESY Formula (XXIII)
that comprises the peptide compound of SEQ ID NO: 15 wherein each lysine residue has a docetaxel molecule connected thereto.

76. The method of claim 73, wherein the anticancer agent is doxorubicin.

77. The method of claim 76, wherein the conjugate compound is represented by formula (XXVI) or (XXVIII):
GVRAK(doxorubicin)AGVRN(Nle)FK(doxorubicin)SESY Formula (XXVI)
that comprises the peptide compound of SEQ ID NO: 10 wherein each lysine residue has a doxorubicin molecule connected thereto; or
Acetyl-GVRAK(doxorubicin)AGVRN(Nle)FK(doxorubicin)SESY Formula (XXVIII)
that comprises the peptide compound of SEQ ID NO: 15 wherein each lysine residue has a doxorubicin molecule connected thereto.

78. The method of claim 73, wherein the anticancer agent is aldoxorubicin.

79. The method of claim 78, wherein the conjugate compound is represented by the formula (LI) or (LII):
GVRAKAGVRN(Nle)FKSESYC(aldoxorubicin) Formula (LI)
that comprises the peptide compound of SEQ ID NO: 23 wherein cysteine residue has an aldoxorubicin molecule connected thereto, or
that comprises the peptide compound of SEQ ID NO: 10 wherein a cysteine residue is added to C-terminal of said peptide compound, and wherein the cysteine residue has an aldoxorubicin molecule connected thereto; or
Acetyl-GVRAKAGVRN(Nle)FKSESYC(aldoxorubicin) Formula (LII)
that comprises the peptide compound of SEQ ID NO: 24 wherein cysteine residue has an aldoxorubicin molecule connected thereto, or
that comprises the peptide compound of SEQ ID NO: 15 wherein a cysteine residue is added to C-terminal of said peptide compound, and wherein the cysteine residue has an aldoxorubicin molecule connected thereto.

80. The method of claim 72, wherein the phytochemical agent is curcumin.

81. The method of claim 80, wherein wherein the conjugate compound is chosen from compounds of formula (XIV), (XV), (XVI) or (XVII):
GVRAK(curcumin)AGVRN(Nle)FK(curcumin)SESY Formula (XIV)
that comprises the peptide compound having SEQ ID NO: 10 wherein each lysine residue has a curcumin molecule connected thereto;
YK(curcumin)SLRRK(curcumin)APRWDAPLRDPALRQLL Formula (XV)
that comprises the peptide compound having SEQ ID NO: 11 wherein each lysine residue has a curcumin molecule connected thereto;
Acetyl-GVRAK(curcumin)AGVRN(Nle)FK(curcumin)SESY Formula (XVI)
that comprises the peptide compound of SEQ ID NO: 15 wherein each lysine residue has a curcumin molecule connected thereto;
Acetyl-YK(curcumin)SLRRK(curcumin)APRWDAPLRDPALRQLL Formula (XVII)
that comprises the peptide compound of SEQ ID NO: 16 wherein each lysine residue has a curcumin molecule connected thereto.
